(19) United States
(12) Patent Application Publication
(10) Pub. No.: US 2025/0213601 A1
(43) Pub. Date: Jul. 3, 2025

(54) METHODS AND BIOAVAILABLE HIGHLY PERMEABLE COMPOUNDS FOR THE TREATMENT OF VIRAL DISEASES

(71) Applicant: Kirill Didenko, Houston, TX (US)

(72) Inventor: Kirill Didenko, Cancun (MX)

(73) Assignee: Kirill Didenko, Houston, TX (US)

(21) Appl. No.: 18/851,210

(22) PCT Filed: Mar. 27, 2023
(86) PCT No.: PCT/IB2023/052993

§ 371 Date: Sep. 26, 2024

Related U.S. Application Data
(60) Provisional application No. 63/324,087, filed on Mar. 27, 2022.
(60) Provisional application No. 63/398,038, filed on Aug. 15, 2022.

(51) Int. Cl. A61K 31/7048; A61K 9/00; A61K 9/14; A61P 31/14

(52) U.S. Cl. CPC A61K 31/7048; A61K 9/0053; A61K 9/146; A61P 31/14

(57) ABSTRACT

Provided are methods and compounds for treating (or for use in manufacturing a pharmaceutical dosage form to treat) viral infections by administering avermectin-based bioavailable highly permeable compounds. The compounds, compositions, and methods provided are particularly useful for the treatment of SARS-CoV-2, dengue, chikungunya, yellow fever, Zika and other viral infections.

### CROSS-REFERENCES TO RELATED APPLICATIONS

This patent application claims the benefit under 35 U.S.C. § 119(e) of U.S. Provisional Patent Application No. 63/324,087 filed on Mar. 27, 2022, and U.S. Provisional Patent Application No. 63/398,038, filed on Aug. 15, 2022. The foregoing applications are incorporated herein by reference in their entireties.

### FIELD OF THE INVENTION

The invention relates generally to methods and compounds for treating viral infections (or for use in manufacturing a pharmaceutical dosage form to treat), particularly methods and compounds based on avermectins for treating Coronaviridae SARS-CoV-2, Flaviviridae Dengue and Zika, and Togaviridae Chikungunya viral infections.

### BACKGROUND OF THE INVENTION

Human coronaviruses, first identified in the mid-1960s, are common viruses that infect most people at some time in their life, generally causing mild to moderate upper respiratory and gastrointestinal tract illnesses. The novel coronavirus referred to as “Middle East Respiratory Syndrome Coronavirus” (MERS-CoV or MERS) was first reported in Saudi Arabia in 2012 and has spread to several other countries. SARS-CoV, the coronavirus responsible for Severe Acute Respiratory Syndrome (SARS) was first recognized in China in 2002 and led to a worldwide outbreak in 2002 and 2003.

Severe acute respiratory syndrome coronavirus 2 (SARS-CoV-2) is a strain of coronavirus that causes COVID-19 (coronavirus disease 2019), the respiratory illness responsible for the ongoing COVID-19 pandemic. First identified in the city of Wuhan, Hubei, China, the World Health Organization declared the outbreak a Public Health Emergency of International Concern on 30 Jan. 2020, and a pandemic on 11 Mar. 2020. SARS-CoV-2 is a positive-sense single-stranded RNA virus that is contagious in humans.

In December 2021, the number of cases continued to climb due to several factors, including new COVID-19 variants. As of that 28 December, 282,790,822 individuals worldwide had been confirmed as infected. As of 14 Apr. 2022, over 500 million cases were confirmed globally. Most cases are unconfirmed, with the Institute for Health Metrics and Evaluation estimating the true number of cases as of early 2022 to be in the billions.

According to the World Health Organization (WHO) in recent years, the growing recognition of Aedes-borne viruses particularly chikungunya virus, zika virus, as well as dengue virus has become a global concern across the world. Unfortunately, due to lack of vector-borne disease surveillance, there is no a comprehensive knowledge about the arboviral diseases, accordingly these are lurking behind the malaria. Underestimation of arboviral diseases may lead to misdiagnosis as well as unnecessary health care expenditures, especially in febrile illnesses suspicious to malaria. Approximately 3.9 billion people from 120 different territories appear to be at risk from these three major arboviruses.

### SUMMARY OF THE INVENTION

### Technical Problem

Some compounds show antiviral activity in vitro, some of them are used in the treatment of diseases, while the effectiveness of the treatment is sometimes insufficient. There is a problem of increasing the bioavailability and permeability of drugs.

Ivermectin is a medication used to treat parasite infestations. In vitro, ivermectin has antiviral effects against several distinct RNA and DNA viruses. In vitro ivermectin could inhibit the replication of RNA viruses such as Zika, dengue, yellow fever, West Nile, Hendra, Newcastle, Venezuelan equine encephalitis, chikungunya, Semliki Forest, Sindbis, Avian influenza A, Porcine Reproductive and Respiratory Syndrome, Human immunodeficiency virus type 1, and severe acute respiratory syndrome coronavirus 2. In vitro ivermectin could inhibit the replication of DNA viruses such as Equine herpes type 1, BK polyomavirus, pseudorabies, porcine circovirus 2, and bovine herpesvirus 1. In vitro, ivermectin could inhibit the replication of severe acute respiratory syndrome coronavirus 2 with EC 50 2.4 μM and EC 90 5 μM.

Documented antiviral action of ivermectin:

| Virus | Inhibitory Concentration/Fold reduction |
|---|---|
| SARS-CoV-2 | EC50 = 2.2/2.8 μM/5000-fold reduction |
| HIV-1 (VSV-G-pseudotyped | 50 μM > 2-fold reduction |
| NL4-3.Luc.R-E-HIV) | |
| Influenza VLPs (avian influenza A) | 10 μM total inhibition |
| Yellow fever virus (17D) | EC50 = 5/0.5 nM/3 PM > 50,000-fold |
| | reduction |
| Dengue virus (EDEN-1) | EC50 = 2.3/3 μM/EC50 = 0.7 μM |
| Dengue virus (NGC) | EC50 = 0.4/0.6 μM/50 μM total inhibition |
| Dengue virus (EDEN-2) | EC50 = 2.1/1.7 μM |
| Dengue virus (EDEN-3) | EC50 = 1.7 μM |
| Dengue virus (EDEN-4) | EC50 = 1.9 μM |
| West Nile virus (NY99) | EC50 = 4 μM |
| West Nile virus (MRM61C) | EC50 = 1/0.5 μM |
| Zika virus (Asian/Cook Islands) | EC50 = 1.3/1.6 μM |
| Chikungunya virus (CHIKV-Rluc) | EC50 = 1.9/0.6 μM/3 μM > 5000-fold |
| | reduction |
| Sindbis (HR) | 3 μM > 1000-fold |
| Semlicki Forest Virus | 3 μM > 200-fold |
| Venezuelan equine encephalitis virus | 1 μM 20-fold |
| (TC83) | |
| Hendra (Hendra virus/Australia/Horse) | est. EC50 = 2 μM |
| Adenovirus (HAdV-C5) | EC50 = 2.5 μM; 10 μM 20-fold reduction |
| Adenovirus (HAdV-B3) | 10 μM 8-fold reduction |
| BK polyomavirus (BKPyV) | est. EC50 1.5 μM |
| Pseudorabies virus | est. EC50 c. 0.8 μM 1000-fold |

The degree of inhibition of virus replication by ivermectin depends directly on the concentration of ivermectin in the body cells. Once the threshold concentration of ivermectin in the cells is reached, each subsequent increase in the concentration of ivermectin in the tissues of the body increases the inhibition. Generally, body tissues have a higher concentration of ivermectin than plasma. As an example, the plasma-to-lung ratio is approximately 2.67 for cattle, and 3.0 for human.

Even a high dose of the standard tablet form of ivermectin of 600 ag/kg/day during for 5 days has been shown to produce a lung Cmax of 0.4-0.9 μM, which is much less than necessary to inhibit viral replication and do not provide the concentration of ivermectin in body tissues necessary to effectively inhibit viral replication.

The technical problem is to provide a method for treating viral diseases in a human or mammal or animal or bird in need thereof comprising administering a therapeutically effective amount of a bioavailable highly permeable avermectin-based compound.

### Solution to Problem

Provided are methods and compounds for the treatment (or for use in manufacturing a pharmaceutical dosage form to treat) of viral diseases comprising administering a therapeutically effective amount of compounds. Provided is a method for the treatment of viral diseases in a human or mammal or animal or bird in need thereof comprising administering a therapeutically effective amount of a compound of FORMULA 1, that is a bioavailable highly permeable solid dispersion (or solutions or suspensions thereof), where the compound of FORMULA I is obtained by co-treating API (active pharmaceutical ingredient guest drug compound) with a HOST substance,
wherein the API is selected from avermectins/ivermectins and their derivatives;
wherein the HOST substance is selected from:
a) polymers and oligomers and macrocyclic hosts, predominantly organic polymers and oligomers, even more predominantly polysaccharides and oligosaccharides, hemicelluloses, storage polysaccharides, sulfated polysaccharides and oligosaccharides, pectins, gums, mucilages, or mixtures thereof;
b) substances that may contain in significant amounts polysaccharides and oligosaccharides, hemicelluloses, storage polysaccharides, sulfated polysaccharides and oligosaccharides, pectins, gums, mucilages, which may be (but are not limited to) plants or algae or animal or fungi, parts of plants or algae or animal or fungi, processed plants or algae or animal or fungi, processed parts of plants or algae or animal or fungi containing in significant amounts the substances specified in paragraph a), or mixtures thereof;
c) syntetic polymers, predominantly water-soluble polymers or mixtures thereof;
d) polyols or mixtures thereof;
e) saccharides or mixtures thereof;
f) surfactants or mixtures thereof;
g) acids or mixtures thereof;
h) oxides and salts based on these oxides or mixtures thereof;
i) copolymers of the polymers listed in paragraph a) and c)
j) methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in a)-i), and their esters, salts, and any other chemical derivatives, and mixtures thereof;
k) any combination of substances specified in paragraph a)-j);
wherein co-treating methods are selected from the methods of obtaining of solid dispersions, including methods of obtaining solid dispersions such as solvent evaporation methods, cryogenic methods, melting methods, mechanochemical methods and other methods, as a non-limiting example, methods selected from can be used:
a) grinding/milling with high energy stress method;
b) grinding/milling with high energy stress and solvent method;
c) media milling method;
d) kneading method;
e) hot-melt method/melting method/fusion method;
f) hot-melt extrusion/hot-stage extrusion method;
g) meltrex method;
h) melt agglomeration method;
i) high-pressure homogenization method;
j) solvent evaporation method;
k) spin-coated films method;
I) spray-drying method;
m) supercritical fluid (SCF) process method;
n) cryogenic techniques method;
o) lyophilization/freeze-drying technique method;
p) spray freezing onto cryogenic fluids method;
q) spray freezing into cryogenic liquids (SFL) method;
r) spray freezing into vapor over liquid (SFV/L) method;
s) ultra-rapid freezing method;
t) precipitation/co-precipitation method;
u) microwave irradiation method;
v) energy input method;
w) heat/shear energy input method;
x) combined method.

In another aspect provided is a method for the treatment treatment (or for use in manufacturing a pharmaceutical dosage form to treat) of viral diseases in a human or mammal or animal or bird in need thereof comprising administering a therapeutically effective amount of a compound of FORMULA II (as well as their solutions, gels, colloids, sols, etc.), that is a bioavailable highly permeable noncovalent complex, which is obtained by adding solvent to the bioavailable highly permeable solid dispersion of FORMULA 1, where the compound of FORMULA I is obtained by co-treating API (active pharmaceutical ingredient guest drug compound) with a HOST substance,
wherein the solvent is usually water;
wherein the molecular information for the noncovalent complex of FORMULA 11 is encoded by the formula (the noncovalent complex of FORMULA 11 is):

GUEST (SOLVENT)m [HOST]n
wherein m is 1 to 100;
wherein n is 1 to 1000;
where GUEST molecular information is given based on the InChl derived from the InChlKey indicated for each of the guest drugs;
wherein SOLVENT molecular information is H2O if solvent is water;

In another aspect provided a medical dosage form of FORMULA I of enhanced bioavailability and permeability characterised in that it comprises a bioavailable highly permeable solid dispersion or aqueous solutions or suspensions thereof, where the compound of FORMULA I is obtained by co-treating API (active pharmaceutical ingredient guest drug compound) with a HOST substance.

In another aspect provided a medical dosage form of FORMULA II (as well as their solutions, gels, colloids, sols, etc.), that is a bioavailable highly permeable noncovalent complex, which is obtained by adding solvent to the bioavailable highly permeable solid dispersion of FORMULA 1, where the compound of FORMULA I is obtained by co-treating API (active pharmaceutical ingredient guest drug compound) with a HOST substance.

In another aspect provided a method for preparing or manufacturing a medical dosage form of FORMULA I of enhanced bioavailability and permeability characterised in that it comprises bioavailable highly permeable solid dispersion or aqueous solutions or suspensions thereof where the compound of FORMULA I is obtained by co-treating API with a HOST substance.

In another aspect provided a method for preparing or manufacturing a medical dosage form of FORMULA II (as well as their solutions, gels, colloids, sols, etc.), that is a bioavailable highly permeable noncovalent complex, which is obtained by adding solvent to the bioavailable highly permeable solid dispersion of FORMULA 1, where the compound of FORMULA I is obtained by co-treating API with a HOST substance.

In another embodiment, the method comprises administering a therapeutically effective amount of a combination pharmaceutical agent (of FORMULA Ill) comprising:
a) a first pharmaceutical composition comprising a compound of FORMULA 1-11, and
b) a second pharmaceutical composition comprising at least one additional therapeutic agent active against viruses or agent that increases the antiviral activity of the FORMULA 1-11 component, or decreases the hepatotoxicity of the FORMULA 1-11 component, or increases the stability of the FORMULA 1-11 component.

In another embodiment, the method comprises administering a therapeutically effective amount of a combination pharmaceutical agent of FORMULA IV in form of solution/emulsion/suspension of comprising:
a) a first pharmaceutical composition comprising a compound of FORMULA 1-111, and
b) at least one of the following components:
(1) oil phase containing vegetable or/and animal fats;
(2) one or more surfactants;
(3) one or more solvents;
(4) one or more gelling agents.

In another embodiment, the method comprises administering a therapeutically effective amount of a combination pharmaceutical agent of FORMULA V in form of food product or beverage, or dietary supplement.

In another embodiment, the method for the treatment of viral diseases in a human or mammal or animal or bird in need thereof comprises administering a therapeutically effective amount of a pharmaceutical composition comprising an effective amount of a FORMULA I-V compound in combination with a pharmaceutically acceptable diluent or carrier.

In another embodiment, the method for the treatment of viral diseases in a human or mammal or animal or bird in need thereof comprises administering a therapeutically effective amount of a pharmaceutical composition comprising an effective amount of a FORMULA I-V compound in combination with at least one additional therapeutic agent.

In another embodiment, the method comprises administering a therapeutically effective amount of compound of FORMULA I-V to a human or mammal or animal or bird in need thereof or use of a compound of FORMULA I-V for the treatment of diseases in a human or mammal or animal or bird.

### Advantageous Effects of Invention

The advantageous effect of the patented invention provides a method for the treatment of viral diseases in a human or mammal or animal or bird or method of manufacturing a pharmaceutical dosage form to treat.

### BRIEF DESCRIPTION OF DRAWINGS

FIG. 1 An example of the chemical formula of one of the noncovalent complexes of FORMULA II.

FIG. 2 Pharmacokinetic parameters after oral administration of Ivermectin and compound of FORMULA I.

FIG. 3 Blood oxygen saturation levels without treatment and after oral administration of compound of FORMULA I.

### DETAILED DESCRIPTION OF THE INVENTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the claims.

### I. DEFINITIONS

The term “treatment” as used herein, unless otherwise specified, means the abolition, relief, inhibition of the progression or prevention of the disorder or condition to which the term applies, or one or more symptoms of such disorder or condition. The term “treatment”, as used herein, refers to the action of treatment as “treating” is defined directly above. Treatment also means “for use in manufacturing a pharmaceutical dosage form to treat”.

The term “therapeutically effective amount”, as used herein, is the amount of a compound of FORMULA I-V present in a composition described herein that is necessary to provide the desired level of the drug in the secretions and tissues of the respiratory tract and lungs or, alternatively, in the bloodstream of the subject to be treated to produce the expected physiological response or desired biological effect when such composition is administered in the selected route of administration. The exact amount depends on a variety of factors, such as the specific compound of FORMULA I-V, the specific activity of the composition, the delivery device used, the physical characteristics of the composition, its intended use, and patient considerations such as severity of the disease state, patient cooperation, etc., and can be readily determined by a person skilled in the art based on the information provided herein.

The term “API” (active pharmaceutical ingredient, active pharmaceutical ingredient guest drug compound, GUEST, guest, guest drug, guest drug compound, guest compound, guest extract, guest biologically active extract) is pharmaceutically active and bioactive compound; if the selected API is in the form of a free base—the pharmaceutical salts of that base and the pharmaceutical ester or ether are also included; if the API is in the form of a pharmaceutical salt—the free base of that salt, the pharmaceutical ester or ether, and other pharmaceutical salts of that free base are also included; also include racemate, enantiomer, diastereomer, tautomer, polymorph, pseudopolymorph, hydrate, hydride, isotopically labeled forms, prodrugs thereof or mixtures thereof.

The term “derivative” of API (guest, guest drug, guest drug compound, guest compound) means derivatives such as racemate, enantiomer, diastereomer, tautomer, polymorph, pseudopolymorph, hydride, hydrate, isotopically labeled forms, prodrugs, pharmaceutical salts, pharmaceutical ester or ether. If the API in the form of a free base, the derivatives are the pharmaceutical salts of that base and the pharmaceutical ester or ether. If the API is in the form of a pharmaceutical salt—the free base of that salt, the pharmaceutical ester or ether, and other pharmaceutical salts of that free base are also derivatives. Derivatives can also be other obvious derivatives.

The term “prodrug” is defined in the pharmaceutical field as a biologically inactive derivative of a API that, when introduced into the human body, is transformed into a biologically active drug according to some chemical or enzymatic pathway.

The term “avermectin” (IVM), as used herein, means avermectins and their derivatives such as: ivermectins and avermectins (A1a, A1b, A2a, A2b, B1a, B1b, B2a, B2b) or racemate, enantiomer, diastereomer, tautomer, polymorph, pseudopolymorph, hydride or solvate thereof or a pharmaceutically acceptable salt or ester thereof or prodrug thereof or mixtures thereof.

### II. COMPOUNDS OF THE PRESENT INVENTION

Reference will now be made in detail to certain embodiments of the invention, examples of which are shown in the accompanying description, structures, and formulas. Although the invention will be described in connection with the embodiments listed, it will be understood that they are not intended to limit the invention to these embodiments. Rather, the invention is intended to encompass all alternatives, modifications, and equivalents that may be included within the scope of the present invention.

The compounds of the present invention may also exist in the form of physiologically acceptable salts. Examples of physiologically acceptable salts include salts derived from an appropriate base such as an alkali metal or alkaline earth metal (e.g., Na+, K+, Ca+2, Mg+2, Li+), ammonium and NR4+(where R is defined herein). Physiologically acceptable salts of a nitrogen atom or amino group include (a) acid accession salts formed with inorganic acids, such as hydrochloric acid, hydrobromic acid, sulfuric acid, sulfamic acid, phosphoric acid, nitric acid and the like; (b) salts formed by organic acids, such as, for example, acetic acidtartaric acid, maleic acid, fumaric acid, lysine, arginine, glutamic acid, glycine, serine, threonine, alanine, iso-leucine, leucine and the like, oxalic acid, salicylic acid, gluconic acid, citric acid, malic acid ascorbic acid, benzoic acid, succinic acid, isethionic acid, lactobionic acid, tannic acid, palmitic acid, alginic acid, malonic acid, naphthalenesulfonic acid, methanesulfonic acid, p-toluenesulfonic acid, naphthalenedisulfonic acid, polygalacturonic acid, sulfosalicylic acid, glycolic acid, 2-hydroxy-3-naphthoate, pamoate, stearic acid, phthalic acid, mandelic acid, lactic acid, ethanesulfonic acid, and (c) salts formed from element anions, e.g., chlorine, bromine, and iodine. Physiologically acceptable salts of a hydroxy group compound include the anion of said compound in combination with a suitable cation such as Na+ and NR4+. All such forms are contemplated within the scope of the invention.

Compounds of the invention have the ability to exist as various polymorphs or pseudopolymorphs. As used in this document, crystalline polymorphism refers to the ability of a crystalline compound to exist in different crystal structures. Crystalline polymorphism can result from differences in crystal packing (packing polymorphism) or from differences in packing between different conformers of the same molecule (conformational polymorphism). As used herein, crystalline pseudopolymorphism refers to the ability of a compound hydrate or solvate to exist in different crystal structures. Pseudopolymorphs of the present invention may exist because of differences in crystal packing (packing pseudopolymorphism) or because of differences in packing between different conformers of the same molecule (conformational pseudopolymorphism). All such forms are considered to be within the scope of the invention.

The compounds of the invention can also exist as amorphous solids. As used herein, an amorphous solid is a solid in which there is no long-range ordering of atoms in the solid. This definition also applies when the crystal size is two nanometers or less. Additives, including solvents, can be used to create the amorphous forms of the present invention. All such forms are contemplated within the scope of the invention.

For therapeutic use, the active ingredients of the compounds of the invention will be physiologically acceptable. Ingredients that are not physiologically acceptable, however, may also find use, such as in the preparation or purification of a physiologically acceptable compound.

Lastly, it is to be expected that the compositions presented herein include the compounds of the invention in their non-ionized as well as zwitterionic forms, and combinations with stoichiometric amounts of water, as in hydrates. All such forms are contemplated within the scope of the invention.

It should be noted that all enantiomers, diastereomers and racemic mixtures, tautomers, polymorphs, pseudopolymorphs of compounds are included in the scope of the present invention. All mixtures of such enantiomers and diastereomers are included in the scope of the present invention.

The compounds of the invention can have chiral centers, such as chiral carbon or phosphorus atoms. Thus, the compounds of the invention include racemic mixtures of all stereoisomers, including enantiomers, diastereomers, and atropisomers. Additionally, the compounds of the invention include enriched or resolved optical isomers on any or all asymmetric chiral atoms. Other words, the chiral centers evident from the figures are presented as chiral isomers or racemic mixtures. Racemic and diastereomeric mixtures as well as individual optical isomers isolated or synthesized essentially free from their enantiomeric or diastereomeric partners are within the scope of the invention. Racemic mixtures are divided into their individual, essentially optically pure isomers by well-known methods, such as, for example, separation of diastereomeric salts formed with optically active excipients, such as acids or bases, followed by conversion back to optically active substances. In most cases, the desired optical isomer is synthesized by stereospecific reactions starting from the corresponding stereoisomer of the desired starting substance. All such forms are provided within the scope of the invention.

The compounds of the invention may in some cases exist as tautomeric isomers. While only one delocalized resonance structure may be depicted, all such forms are considered within the scope of the invention.

Any formula or structure herein is also intended to represent unlabeled forms and isotopically labeled forms of compounds. Isotopically labeled compounds have the structures represented in the formulas provided herein except that one or more atoms are replaced with an atom of a selected atomic mass or mass number. Examples of isotopes that may be included in the compounds of the disclosure include isotopes of hydrogen, carbon, nitrogen, oxygen, phosphorus, fluorine and chlorine such as 2H (deuterium, D), 3H (tritium), SS, 13C, 14C, 1SN, 18F, 31P, 32P, 35S, 36C1 and 125I, but without limitation. Different isotopically labeled compounds of the present disclosure, such as those incorporating radioactive isotopes such as 3H, 13C and 14C. All such forms are provided within the scope of the invention. Such isotopically labeled compounds can be useful in metabolic studies, reaction kinetic studies, detection or imaging techniques such as positron emission tomography (PET) or single photon emission computed tomography (SPECT), including tissue distribution analyses of drugs or substrates, or in the radioactive treatment of patients. The disclosure also includes compounds of FORMULA I in which 1 to n hydrogens attached to the carbon atom are substituted with deuterium, where n is the number of hydrogens in the molecule. These compounds exhibit increased resistance to metabolism and thus are useful for increasing the half-life of any compound of FORMULA I when administered to a mammal, particularly a human.

### III. KEY METHODS AND CONCEPTS OF INVENTION

Solid dispersion of the invention can be defined as the dispersion of one or more active ingredients (API) in an inert carrier or matrix (HOST) at solid state prepared by the melting/fusion, solvent, melting-solvent method, mechanochemical treatment and other methods. The API drug is hydrophobic in nature whereas matrix (HOST) is hydrophilic. Solid dispersions of the present invention may be in the form of molecular dispersions, simple eutectic mixtures, solid solutions, glass solutions and glass suspensions, mechanochemically obtained solid dispersions (including solid dispersions in the form of micro-mechano-composites in which solid-phase fusion, mechanosynthesis, mechanoactivation processes have occurred), amorphous precipitation in a crystalline carrier, compound or complex formations. Solid dispersions of the present invention may be in the form of first generation solid dispersions; second generation solid dispersions; third generation solid dispersions; fourth generation solid dispersions. Solid dispersions of the present invention may be in the form of: Class C-C, Class C-A, Class A-C, Class A-A, Class M-C, Class M-A based on physical state and molecular arrangement of both API and carrier.

TABLE 1
Classification According to Physical State and
Molecular Arrangement of API and Carrier (HOST)

| Class | API (API) | Carrier (HOST) |
|---|---|---|
| C-C | Crystalline | Crystalline |
| C-A | Crystalline | Amorphous |
| A-C | Amorphous | Crystalline |
| A-A | Amorphous | Amorphous |
| M-C | Molecularly dispersed | Crystalline |
| M-A | Molecularly dispersed | Amorphous |

Many analytical and instrumental technique are used to characterize solid dispersions. Techniques used for characterization are can be thermal methods, spectroscopic methods, microscopic methods, microthermal analysis, macroscopic techniques, etc. Just a mixture of substances, without treatment of the API and carrier (HOST), does not lead to the results that solid dispersion leads to. Solid dispersion is not a mixture of synergists that amplify each other's effects. Solid dispersion is not a type of nanoparticle.

Noncovalent complex of the invention are molecular ensembles that, as a whole, have specific properties that differ from the environment and whose internal structure and form are determined by non-covalent interactions of atoms. This distinguishes noncovalent systems from molecular systems in which the structure is determined mainly by covalent interactions. Noncovalent complex are molecular complexes of two or more cationic, anionic or neutral molecules held together in a single system by hydrogen bonding, van der Waals interactions, donor-acceptor interactions, n-T interactions, ionic interactions, hydrophobic interactions. Noncovalent complex have the ability to form different architectures while the chemical composition of the system remains unchanged. Noncovalent complex of the invention consisting of at least two different components, generally a matrix (HOST, HOST substance, “host”, “host substance”, “carrier”, etc.) and a therapeutic agent (API, active pharmaceutical ingredient, drug, guest, “guest drug”, “guest drug compound”, “guest substance”, etc.). Many analytical and instrumental technique are used to characterize noncovalent complexes (spectroscopic methods, etc.).

Provided is a method of treatment (and for use in manufacturing a pharmaceutical dosage form to treat) of the disease by administering a therapeutically effective amount of the solid dispersion to a human or mammal or animal or bird in need thereof comprising: contacting an API and a matrix “HOST substance” to form a solid dispersion under conditions sufficient to form a solid dispersion; and (optionally) dissolving solid dispersion in solvent to form the liquid solubilized noncovalent complex (to form solutions, gels, colloids, sols, suspensions, etc.) under conditions sufficient to form the liquid solubilized noncovalent complex.

In another aspect provided is a method of treatment (and for use in manufacturing a pharmaceutical dosage form to treat) of the disease by administering a therapeutically effective amount of the solid dispersion or the noncovalent complex to a human or mammal or animal or bird in need thereof wherein the “carrier substance” (HOST substance) molecule of the solid dispersion or the noncovalent complex interacts with the biological membranes of the organism in such a way that the transfer of molecules and ions of API across this biological membrane is improved; permeability of API is improved in the form of the noncovalent complex. Thus, the scope of the invention includes solid dispersions and noncovalent complexes with special permeability properties; solid dispersions and noncovalent complexes with high permeability-highly permeable solid dispersions and noncovalent complexes.

In another aspect provided is a method of treatment (and for use in manufacturing a pharmaceutical dosage form to treat) of the viral disease by administering a therapeutically effective amount of the solid dispersion or the noncovalent complex to a human or mammal or animal or bird in need thereof wherein the solid dispersion or the noncovalent complex of API has a higher bioavailability than the API itself is presented.

In another aspect provided is a method of treatment (and for use in manufacturing a pharmaceutical dosage form to treat) of the viral disease by administering a therapeutically effective amount of the solid dispersion or the noncovalent complex to a human or mammal or animal or bird in need thereof wherein the solid dispersion or the noncovalent complex of API has a lower toxicity and cytotoxicity than the API itself is presented.

In another aspect provided is a method of treatment (and for use in manufacturing a pharmaceutical dosage form to treat) of the viral disease by administering a therapeutically effective amount of the solid dispersion or the noncovalent complex to a human or mammal or animal or bird in need thereof wherein the solid dispersion or the noncovalent complex of API has a different pharmokinetic parameters than the API itself is presented.

In another aspect provided is a method of preparing or manufacturing the solid dispersion or the noncovalent complex of the invention.

The methods for obtaining solid dispersions of the present invention include:
1) solvent evaporation and cryogenic methods;
2) melting methods and hot-stage extrusion methods;
3) mechanochemical methods (but not excluding other methods).
   Particle size of solid dispersion may vary and depends on the equipment device (e.g. nozzle size), equipment operation parameters, the degree of grinding of the finished product. The usual particle size is 0.5-1000 microns, but can exceed 1000 microns, and the resulting solid dispersion can also be ground to submicron size after it is obtained.

The solvent evaporation method consists of the solubilization of the API and carrier (HOST substance) in a volatile solvent that is later evaporated. In this method, the thermal decomposition of API or carriers can be prevented, since organic solvent evaporation occurs at low temperature. Differences in solvent evaporation processes are related to the solvent evaporation procedure, which usually include vacuum drying, heating of the mixture on a hot plate, slow evaporation of the solvent at low temperature, the use of a rotary evaporator, a stream of nitrogen, spray-drying, freeze-drying, the use of supercritical fluids (SCF) and other types of solvent evaporation.

Melting method consisting of melting the API within the carrier (HOST substance) followed by cooling and pulverization of the obtained product. In the melting process, the molecular mobility of carrier is high enough to change the drug's incorporation. A common adaptation to the melting phase consists of suspending the API in a previously melted carrier, instead of using both drug and carrier in the melted state, reducing, therefore, the process temperature. To cool and solidify the melted mixture, several processes such as ice bath agitation, stainless steel thin layer spreading followed by a cold draught, solidification on petri dishes at room temperature inside a dessicator, spreading on plates placed over dry ice, immersion in liquid nitrogen or stored in a dessicator, and other types of cooling were used. After cooling, the mixture must be pulverized regarding its handling. However, the use of high temperatures, and the fact that several drugs can be degraded by the melting process, can be a limitation of this method. The incomplete miscibility between drug and carrier that may occur, because of the high viscosity of a polymeric carrier in the molten state, is another limitation of this process. To avoid the melting method limitations, several modifications, like hot-stage extrusion, Meltrex, melt agglomeration or other were introduced to the original method.

The mechanochemical method of obtaining solid dispersions consists in intensive mechanical processing (for example, in planetary or other types of mills) under conditions where the processes of solid-phase fusion, mechanosynthesis, mechanoactivation occur between the components, including the formation of microcomposites, in which the processes of mechanochemical solid-phase fusion/melting of the API with the HOST substance, mechanosynthesis, mechanoactivation have occurred, when the mixing of components results in a reaction in the solid phase, forming products that have high contact surface and extremely high concentration of defects of various kinds. The formation of the interfacial surface, which is necessary for the realization of physic-chemical interaction, takes place. Under these conditions, the characteristic time of mass transfer of reagents to each other for the passage of the physio-chemical reaction decreases. Simultaneously with comminution and formation of mechanochemically obtained solid dispersions, additional structural defects are created in the mixture components. In the macroscopic approximation, the activation barrier of chemical interaction decreases. As a rule, the longer the mechanical processing time, the higher the bioavailability and permeability of the resulting solid dispersions, while the particle size can remain practically unchanged.

At the initial stage of mechanochemical processing the initial collisions of powder particles with balls lead to plastic deformation of particles and their flattening. Continued plastic deformation is accompanied by an increase in the ratio of the surface area of the particles to their volume. In the process of deformation of the particles the pure inner layers of the API are opened, which come into close contact with the pure layers of the other component, causing the process of welding. The accompanying plastic deformation hardening reaches a critical value and the formed object is destroyed. The three processes—plastic deformation, welding and fracture of the machined particles—are then repeated many times in parallel. In the process, the material acquires a layered, twisted structure. At the end of the initial fusion stage, a mixed heterophase system called a mechanocomposite is formed. The mechanocomposite has a morphologically metastable structure with a high density of interphase boundaries between the initial components, which provide a developed contact surface and a high concentration of defects due to the large number of atoms on the surfaces and in the near-surface layers. Such a system has a large stored energy, which, together with the extremely large contact surface between the components, provides a high reactivity of the system. At the same time, despite the almost perfect contact surface of the reagents in the matrix, large inclusions of the more brittle component in contact with the plastic reagent can be observed in the volume of the mechanocomposite.

The method of mechanochemical processing leads to the same results as the other methods, but excludes toxic solvents or thermal degradation processes of the API.

In the present invention, bioavailable solid dispersions and bioavailable noncovalent complexes are obtained and used. Bioavailable with respect to the solid dispersions and noncovalent complexes means at least one of the following:
1) absolute, relative bioavailability of the solid dispersion or noncovalent complex of the API is higher than that of the API itself;
2) concentrations, Cmax, AUC and other pharmokinetic parameters of API and/or its metabolites in blood, organs or tissues for the solid dispersion or noncovalent complex of the API is higher than that of the API itself.
   The rate of release of API molecules from noncovalent complexes can play a significant role in increasing bioavailability.

Noncovalent complexes play a particularly important role in biological systems, such as the transfer of molecules and ions across biological membranes. Highly permeable solid dispersions or noncovalent complexes are obtained and used in the current invention. Highly permeable with respect to the solid dispersions or the noncovalent complexes of the present invention means at least one of them:
1) the transfer of molecules, ions, metabolites of API across the biological/cell membrane is improved for the solid dispersion or the noncovalent complex of API than for the API itself is presented;
2) the transfer of molecules, ions, metabolites of API across the Caco-2 cell line layer or other cell line layer (the intestinal epithelium cells layer, etc.) is improved for the solid dispersion or the noncovalent complex of API than for the API itself is presented;
3) intestinal permeability is improved for the solid dispersion or the noncovalent complex of API than for the API itself is presented;
4) increase in permeability according to the methods used to determine permeability in The Biopharmaceutics Classification System.

Within the scope of the current invention, an increase in permeability and an increase in absorption of an API from solid dispersions or noncovalent complexes is achieved through the following mechanisms (but not excluding other mechanisms for increasing permeability):
1) as a consequence of blocking/reducing the activity of CYP3A4 and/or P-glycoproteins, blocking/reducing the activity of other transmembrane carrier proteins, transporters, enzymes, etc., by membrane interacting with carrier substance (HOST) molecules;
2) due to blocking/reduction of CYP3A4 and/or P-glycoprotein activity, blocking/reduction of activity of other transmembrane protein transporters, transporters, enzymes, etc., by carrier substance (HOST) molecules interacting with the membrane due to adhesion of carrier substance (HOST) molecules to the cellular layer. Such adhesion can lead to interaction with transporters domains located on the outer part of the cell membrane and block the action of the transmembrane transporter protein;
3) due to a change in the structure of the cell membrane after the incorporation of carrier substance (HOST) molecules into this membrane, rather than due to adhesion;
4) due to the direct effect of carrier substance (HOST) molecules on the intestinal epithelium cell membrane: it may consist in alteration of lipid bilayer properties, density of intercellular contacts or inhibition of trans-membrane proteins of multidrug resistance;
5) due to changes in hydrophilicity and hydrophobicity of the non-covalent complex, due to which changes in the paracellular transport of the non-covalent complex may occur;
6) other changes in the structure and properties of the membrane, through other mechanisms that contribute to an increase in its permeability.

Carriers plays major role in formulation of solid dispersion. They can be hydrophilic or hydrophobic or water swellable. Depending on their characteristics they can be used as release retardant or release enhancers. Also the dissolution characteristics of drug molecules are depend on nature of carriers. The criteria for selection of carrier (HOST) are as follows:
1) It should be water soluble or swellable, soluble in variety of solvents.
2) It should be economical, pharmacologically inert, non-toxic.
3) It should be heat stable.
4) Chemically compatible with drug.

Water soluble and non-water soluble carriers must prevent the recrystallization trend and help to maintain the supersaturation state after dissolution (spring and parachute effect) via hydrogen bonding interaction

Polymers those are derived from plant origin are used in the current invention because of their diverse pharmaceutical applications and also easy availability, biocompatibility, non-toxic nature, chemically inertness they are preferred over the synthetic ones. Polysaccharide, one of the most abundant industrial raw materials are used because of their sustainability, bio-safety and bio-degradability. The natural gums are metabolic by-products of plants obtained from various parts of plant like seed, fruit, incised trunk (gummy exudate), etc.

Natural polysaccharides are biodegradable, biocompatible materials for use in drug delivery systems. However, these materials have certain limitations, like uncontrolled rate of hydration, change in viscosity during shelf life, microbial contamination. Therefore to overcome this problems some modification have done. Modifications can be done in terms of physical modifications and chemical modifications. Physical methods for modification of involves use of dry heat, microwave technology, UV and gamma radiations. While chemical modifications involves carboxymethylation/carbomoylethylation in which free —OH groups replacement were done which leads to improved aqueous drug solubility. Generally natural carriers on modification using heating method will leads to change in its physical characteristics like viscosity, density, swelling index, water holding capacity, flow properties etc. Due to changes in these characteristics the improved results were obtained in case of modified natural carriers. Carboxymethylation of natural carriers increases their hydrophilicity and makes them more soluble in aqueous systems.

### III.I. Section

Provided is a method for the treatment (and for use in manufacturing a pharmaceutical dosage form to treat) of viral diseases in a human or mammal or animal or bird in need thereof comprising administering a therapeutically effective amount of a compound of FORMULA I, that is a highly bioavailable highly permeable solid dispersion (or solutions or suspensions thereof), where the compound of FORMULA I is obtained by co-treating API compound with a HOST substance,
wherein a person's medical conditions or diseases or disorders or syndromes are selected from those listed in The International Statistical Classification of Diseases and Related Health Problems (including ICD-9, ICD-10, ICD-10-CM, ICD-11, ICD-11-CM, and others);
and/or wherein a person's medical conditions or diseases or disorders or syndromes are selected from those listed in The Diagnostic and Statistical Manual of Mental Disorders, including DSM-IV, DSM-5, DSM-5-TR, and others;
and/or wherein a person's medical conditions or diseases or disorders or syndromes are selected from those listed in the Medical Subject Headings and beginning with MeSH codes C01-C25;
and/or wherein a person's medical conditions or diseases or disorders or syndromes are selected from conditions that are published in the Genetic and Rare Diseases (GARD) Information Center;
and/or wherein a human medical conditions or diseases or disorders or syndromes/infections are caused by viruses; including those caused by RNA viruses, DNA viruses and reverse transcribing viruses; including those caused by viruses belonging to the realm Duplodnaviria, Monodnaviria, Adnaviria, Ribozyviria, Riboviria, and Varidnaviria; including those caused by dsDNA viruses, ssDNA viruses, dsRNA viruses, (+)ssRNA viruses, (−)ssRNA viruses, ssRNA-RT viruses, dsDNA-RT viruses; including viral infections or viral diseases caused by viruses included in the families Coronaviridae, Flaviviridae, Herpesviridae, Orthomyxoviridae, Paramyxoviridae, Polyomaviridae, Retroviridae, Togaviridae, but not excluding other viruses families; including viral infections or viral diseases caused by Human SARS coronavirus, SARS coronavirus 2, Dengue virus, West Nile virus, Yellow fever virus, Zika virus, Bovine alphaherpesvirus 1, Influenza A virus, Hendra virus, BK polyomavirus, Human immunodeficiency virus type 1, Chikungunya virus, Semliki forest virus, Sindbis virus, Venezuelan equine encephalitis virus, but not excluding other viral infections or viral diseases caused by Human adenovirus, Junin arenavirus, Lassa virus, Lymphocytic choriomeningitis virus, Machupo virus, Pichinde virus, Porcine reproductive and respiratory syndrome virus (PRRSV)/Betaarterivirus suid 1, Human astrovirus, Hantaan virus, Crimean-Congo hemorrhagic fever virus, Dugbe virus, Bunyamwera virus, Bunyavirus La Crosse, Bunyavirus snowshoe hare, Punta toro phlebovirus, Rift valley fever virus, Sandfly fever Naples phlebovirus (Toscana virus), Sandfly fever sicilian virus, Uukuniemi virus, Norwalk virus, Southampton virus, Sapporo virus, porcine circovirus 2, Human coronavirus, MERS coronavirus, Human torovirus, Ebola virus, Lake Victoria marburg virus, Japanese encephalitis virus, Langat virus, Louping ill virus, St. louis encephalitis virus, Tick-borne powassan virus, TBE virus, Hepatitis C virus, GB virus C/Hepatitis G virus, Hepatitis B virus, Hepatitis E virus, Human cytomegalovirus, Cercopithecine herpesvirus, Epstein-Barr virus, Human herpesvirus 8, Human herpesvirus 6, Human herpesvirus 7, Human herpesvirus 1, Human herpesvirus 2, Varicella-zoster virus, Bovine alphaherpesvirus 1, Equine herpesvirus type 1 (EHV-1), Pseudorabies virus, Rubella virus, Influenza B virus, Influenza C virus, Influenza A virus, Dhori virus, Human papillomavirus (16,18), Human papillomavirus 2, Human papillomavirus 1, Nipah virus, Measles virus, Human parainfluenza, Mammalian orthorubulavirus 5 (Simian virus 5), Mumps virus, Newcastle, Adeno-associated virus, Human parvovirus B19, Encephalomyocarditis virus, Cosavirus A, Coxsackievirus, Echovirus, Human enterovirus (68, 70), Poliovirus, Hepatitis A virus, Aichi virus, Rosavirus A, Rhinovirus, Salivirus A, Human respiratory syncytial virus, JC polyomavirus, KI Polyomavirus, Merkel cell polyomavirus, WU polyomavirus, BK polyomavirus, Molluscum contagiosum virus, Cowpox virus, Horsepox virus, Vaccinia virus, Variola virus, Yaba monkey tumor virus, Yaba-like disease virus, Smallpox, Orf virus, Rotavirus A, Rotavirus B, Orbivirus, Coltivirus, Rotavirus C, Banna virus, Human T-lymphotropic virus, Human immunodeficiency virus, Eastern chimpanzee simian foamy virus, Simian foamy virus, Human immunodeficiency virus type 1/2, Australian bat lyssavirus, Duvenhage virus, Lagos bat virus, Mokola virus, Rabies virus, Chandipura virus, Isfahan virus, Vesicular stomatitis virus, European bat lyssavirus, Barmah forest virus, Eastern equine encephalitis virus, Mayaro virus, O'nyong-nyong virus, Ross river virus, Sagiyama virus, Western equine encephalitis virus, Rubella virus, Hepatitis delta virus, Hepatitis E virus, Torque teno virus, Human rhinovirus, Murray valley encephalitis virus, Oropouche virus, Monkeypox virus, New York virus, Puumala virus, Seoul virus, but not excluding other viruses;
wherein the API compound is selected from ivermectins or avermectins and their derivatives (each API has a sequential number and an InChlKey, which are indicated in parentheses after the name of the API (GUEST drug), first the InChlKey and then the sequential number); API is selected from:
ivermectins (SPBDXSGPUHCETR-JFUDTMANSA-N or AZSNMRSAGSSBNP-ZLRQRZFZSA-N, 1); avermectin Ala (AFSHKCWTGFDXJR-SQOHEDJBSA-N, 2); avermectin Alb (MNRHCELBXZARFX-OVBDMLLUSA-N, 3); avermectin A2a (JVGWUGTWQIAGHJ-DFAYUBCLSA-N, 4); avermectin A2b (QUTFLJHOCPQPEW-WUSILSRKSA-N, 5); avermectin Bla (AZSNMRSAGSSBNP-XPNPUAGNSA-N, 6); avermectin B1b (VARHUCVRRNANBD-PVVXTEPVSA-N, 7); avermectin B2a (CWGATOJEFAKFBK-QJDTYEMPSA-N, 8); avermectin B2b (ZPAKHHSWIYDSBJ-YAGODIQJSA-N, 9) or racemate, enantiomer, diastereomer, tautomer, polymorph, pseudopolymorph, hydrate or solvate thereof or a pharmaceutically acceptable salt or ester thereof or prodrug thereof or mixtures thereof; The sequence number is used to abbreviate the API, and InChlKey is used to obtain molecular information about the API. So for example for GUEST3—“3” is the sequential number of compound avermectin Alb, and GUEST3 means avermectin Alb for which InChlKey is MNRHCELBXZARFX-OVBDMLLUSA-N. Based on this InChlKey the molecular information InChl for avermectin Alb (GUEST3) can be derived: InChl=1S/C48H72014/c1-25(2)41-28(5)17-18-47(62-41)23-34-20-33(61-47)16-15-27(4)42(59-39-22-37(53-10)44(31(8)57-39)60-38-21- 36(52-9)40(49)30(7)56-38)26(3)13-12-14-32-24-55-45-43(54-11)29(6)19-35(46(50)58-34)48(32,45)51/h12-15,17-19,25-26,28,30-31,33-45,49,51H,16,20-24H2,1-11H3/b13-12+,27-15+,32-14+/t26-,28-,30-,31-,33+,34-,35-,36-,37-,38-,39-,40-,41+,42-,43+,44-,45+,47+,48+/m0/s1;

Accordingly, GUEST1-GUEST3 means all the guest drug having sequence numbers 1-3, that is: GUEST1, GUEST2, GUEST3.

If the selected API is in the form of a free base—the pharmaceutical salts of that base and the pharmaceutical ester or ether are also included. If the API is in the form of a pharmaceutical salt—the free base of that salt, the pharmaceutical ester or ether, and other pharmaceutical salts of that free base are also included. Also include racemate, enantiomer, diastereomer, tautomer, polymorph, pseudopolymorph, hydrate or prodrug thereof or mixtures thereof.

Thus API is selected from GUEST1-9.

The weight of the API is 0.01-90% of the total weight of FORMULA I.

And wherein the HOST substance is selected from (10-99.99% of the total weight of the FORMULA I):
A. Polymers and oligomers and macrocyclic hosts, predominantly organic polymers and oligomers, even more predominantly polysaccharides and oligosaccharides, hemicelluloses, storage polysaccharides, sulfated polysaccharides and oligosaccharides, pectins, gums, mucilages, which may be (but are not exclusive to):
A.1. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, their mixtures, but not excluding other hemicelluloses, and mixtures thereof;
A.2. Sulfated polysaccharides and oligosaccharides which may be fucoidans, carrageenans or carrageenins, agaropectins, sea cucumber sulfated polysaccharides (SCSP), chondroitin sulfate, keratan sulfate, their mixtures, but not excluding other sulfated polysaccharides and oligosaccharides, and mixtures thereof;
A.3. Polysaccharides and oligosaccharides, storage polysaccharides, sulfated polysaccharides and oligosaccharides, pectins, gums, mucilages which may be (but are not exclusive to others): polysaccharides based on glucose, dextrose, galactose, mannose, arabinose, rhamnose, sucrose, maltose, lactose and their uronic acids, which may be methoxylated or acetylated, and their salts (gum, gummi); polyuronic acids and esters; gum, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups, phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses, galacto-oligosaccharides (oligogalactosyllactose, oligogalactose, oligolactose or transgalactooligosaccharides), xylooligosaccharides, fructooligosaccharides, isomaltooligosaccharide; alginic acid and its salts alginates, propylene glycol alginate; arabin, arabic acid and its salts; cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s) (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch; dextrin(s), maltodextrins, cyclodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid and mixtures thereof, but not excluding other polysaccharides and oligosaccharides, hemicelluloses, stored polysaccharides, sulfated polysaccharides and oligosaccharides, mucilages and mixtures thereof;
A.4. Macrocyclic hosts which may be (but are not exclusive to others): cyclodextrins, cucurbit[n]urils, and calix[n]arenes, pillarenes, crown ethers, cyclophanes, cryptands;
B. Substances that may contain in significant amounts (more than 5% wt.) polysaccharides and oligosaccharides, hemicelluloses, storage polysaccharides, sulfated polysaccharides and oligosaccharides, pectins, gums, mucilages, which may be (but are not limited to) plants or algae or animal or fungi, parts of plants or algae or animal or fungi, processed plants or algae or animal or fungi, processed parts of plants or algae or animal or fungi containing in significant amounts the substances specified in paragraph A, and mixtures thereof. A frequent but non-limiting example is dried brown or red algae such as kelp or focus;
C. Syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s, poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit S100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat(s), Kollicoat IR, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s), Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone S-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-a-tocopherol polyethylene glycol 1000 succinate (TPGS), Isomalt (Galen IQ 810), but not excluding other synthetic polymers and mixtures thereof;
D. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
E. Saccharides which may be glucose, dextrose, galactose, mannose, arabinose, rhamnose, sucrose, maltose, lactose, ribose, and mixtures thereof;
F. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
G. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof;
H. Oxides and salts based on these oxides which may be (but are not limited to): silicon dioxide, silicates, titanium dioxide, and mixtures thereof;
I. Copolymers of the polymers listed in paragraph A, C including alternating copolymers, random copolymers, block copolymers, graft copolymers, cross-linked modifications which may be (but are not limited to):
methacrylic acid and ethyl acrylate copolymers; methacrylic acid copolymers; vinylpyrrolidone-vinyl acetate copolymers (PVPVA); polyvinylpolypyrrolidone (PVPP); PVA-PEG graft co-polymers; HPMC and PVA-PEG grafted copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols orpolymeth-ylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral copolymers based on methacrylic acid and methacrylic/acrylic esters their salts, esters or other derivatives and mixtures thereof;
J. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-I, and their esters and ethers, salts, and any other chemical derivatives, and mixtures thereof;
K. Any combination of substances specified in items A-J;
wherein co-treating method is selected from (but not excluding other methods, which should lead to the formation of the bioavailable solid dispersion):

Method 1 (Grinding or Milling With High Energy Stress; Mechanochemical Method): “API” and the “HOST substance” are milled in a roller, ball, planetary, vibratory, jet, drum, medium-speed, impact-jet, centrifugal countercurrent, rotary, disintegrator, and other types of mills or other grinders. The grinding process may produce particles of a size of mainly 1 to 1000 microns, which are powders. Grinding should be carried out to such a state that there are changes in the structure and properties of the ground substance so that it has increased bioavailability and permeability and bioavailable highly permeable solid dispersion is formed. Grinding should cause structural changes such as defects, increased deformation, associates. In the grinding process any of the processes of solid-phase fusion, mechanosynthesis, mechanoactivation must occur and including the formation of microcomposites in which the processes of mechanochemical solid-phase fusion of the API substance with the HOST substance, mechanosynthesis, mechanoactivation have occurred, when the mixing of components occurs reaction in the solid phase, forming products that have high contact surface and extremely high concentration of defects of various kinds.

A solvent may be added to the grinding process, such as organic and inorganic solvents which may be (but are not limited to):
water, hydrocarbons and their halogen derivatives, alcohols, esters and ethers, ketones, nitro compounds: 1,1,1-trichloroethane; 1,1,2-trichloroethene; 1,1-dichloroethene; 1,2-dichloroethane; 1,2-dichloroethene; 1,2-dimethoxyethane; 1,2-propanedioi, 1,4-dioxane, 1-butanol, 1-pentanol, 1-propanol, 2-butanol, 2-butanone, 2-ethoxyethanol, 2-methoxyethanol, 2-methyl-1-propanol, 2-methylpyridine, 2-methyltetrahydrofuran, 2-nitropropane, 2-propanol, 3-methyl-1-butanol, 4-methyl-2-pentanone, acetic acid, acetic anhydride, acetone, acetonitrile, acetophenone, ammonia, aniline, anisole, benzene, benzonitrile, benzylalcohol, bromoethane, bromoform, bromooctane, butanol, butanone, butylacetate, butylbenzene, carbon disulfide, carbontetrachloride, carbontet, cellosolve acetate, cetic acid, chlorobenzene, chloroform, chloroform, chlorohexane, cumene, cyclohexanes cyclohexane, cyclohexanol, cyclohexanone, cyclopentane, cyclopentanes, decalin, decane, decanol, dibromoethane, dibutylether, dichlorobenzene, difluorobenzene, dichloroethane, dichloromethane, diethyleneglycol, diethylether, diglyme (diethylene glycoldimethyl ether), diisopropylether, dimethoxyethane, dimethyl acetate, dimethyl sulfoxide, dimethylacetamide, dimethylformamide, dimethylpyridine, dimethylsulfoxide, dioxane, dioxanes (1.4 dioxane), dodecane, esters, etbe, ethanediol, ethanol, ether(s), ethoxybenzene, ethyl acetate, ethyl acetoacetate, ethyl alcohol, ethylacetate, ethylbenzene, ethylene glycol ethyl ether, ethylene glycol methyl ether, ethylene glycol monobutyl ether, ethyleneglycol, ethylether, ethylformate, fluorobenzene, fluoroctane, formamide, formic acid, freon, furfuraldehyde, glacial acetic acid, glycerin, glycol ethers, halobenzenes, heptane, heptanes, heptanol, hexadecane, hexadecyliodide, hexamethylphosphoramide, hexamethylphosphorous triamide (hmpt), hexane, hexanes, hexanol, i-amyl alcohol, iodobenzene, isobutanol, isobutyl acetate, isooctane, isopropanol, isopropyl acetate, isopropylbenzene, isopropyltoluene, mcresol, mesitylene, methanoic acid, methanol, methoxyethanol, methyl acetate, methyl ethyl ketone, methyl t-butyl ether, methyl tert butyl ether, methylbutyl ketone, methylcyclohexane, methylenechloride, methylethyl ketone, methylformamide, methylisobutyl ketone, miscellaneous solvents, monochlorobenzene, morpholine, m-xylene, n,n-dimethylacetamide, n,n-dimethylformamide, n-amyl alcohol, n-butyl acetate, nitrobenzene, nitroethane, nitromethane, n-methyl-2-pyrrolidinone(nmp), n-methylpyrrolidone, n-octanol, nonane, nonanol, octane, octanol, odichlorobenzene, onitrotoluene, o-xylene, pentadecane, pentane, pentanes, pentanol, perchloroethylene, perfluorobenzene, petroleum ether (ligroine), phenol, phenylether, propanol, propoxypropane, propyl acetate, propylene glycol methyl ether, p-xylene, pyridine, secbutanol, secbutylbenzene, sulfolane, t-butanol, t-butyl alcohol, tbutylbenzene, tert-butylmethylether, tetrachloroethane, tetrachloroethylene, tetrachloromethane, tetrahydrofuran, tetrahydrothiophenedioxide, tetralin, toluene, tributylphosphate, trichloroethane, trifluoromethylbenzene, trichloroethylene, triethylamine, trimethylbenzene, trimethylpentane, undecane, xylene, xylenes, 2,2,4-trimethyl pentane, and mixtures thereof;

Method 2 (Grinding or Milling With High Energy Stress and Solvent; Mechanochemical Method With Solvent): “API” and the “HOST substance” are wetted with a solvent, the wetted mass is dried and then ground in the same manner as described in Method 1. The list of solvents used is the same as the solvents listed in Method 1;

Method 3 (Media Milling): The solid dispersion is prepared by using high-shear media mills. The milling chamber charged with milling media, solvent, “API”, and “HOST substance” is rotated at a very high-shear rate under controlled temperatures. The milling medium is composed of glass, zirconium oxide, highly cross-linked polystyrene resin or other standard milling medium. High energy shear forces are generated as a result of the impaction of the milling media with the “API” and “HOST substance”. The list of solvents used is the same as the solvents listed in Method 1;

Method 4 (Kneading Method): This method is based on impregnating the “HOST substance” with a little amount of water or hydroalcoholic solutions to convert it into a paste. The “API” is then added to the above paste and kneaded for a specified time. The kneaded mixture is then dried and passed through a sieve if required. On a laboratory scale, kneading can be achieved by using a mortar and pestle. On large scale, kneading can be done by utilizing extruders and other machines;

Method 5 (Hot-Melt Method, Melting Method, Fusion Method): In this method, the physical mixture of “API” and a “HOST substance” are heated directly until the two melt. The melted mixture is then cooled and solidified rapidly with rigorous stirring. The final solid mass is then crushed, pulverized, and sieved. The melting method consists of melting the “API” within the “HOST substance” followed by cooling and pulverization of the obtained product. A common adaptation to the melting phase consists of suspending the “API” in a previously melted “HOST substance”, instead of using both “API” and “HOST substance” in the melted state, reducing, therefore, the process temperature. To cool and solidify the melted mixture, several processes such as ice bath agitation, stainless steel thin layer spreading followed by a cold draught, solidification on petri dishes at room temperature inside a desiccator, spreading on plates placed over dry ice, immersion in liquid nitrogen or stored in a desiccators or other cooling method were used. After cooling, the mixture must be pulverized regarding its handling. KinetiSol® dispersing technology also can be used;

Method 6 (Hot-Melt Extrusion, Hot-stage extrusion): Hot-Melt Extrusion is a modification of the Melting Method. Hot-melt extrusion is essentially the same as the melting method except that intense mixing of the components is induced by the extruder. Hot-stage extrusion consists of the extrusion, at high rotational speed, of the “API” and “HOST substance”, previously mixed, at melting temperature for a small period of time. The resulting product is then collected after cooling at room temperature and milled. A reduction in processing temperature can be achieved by the association of hot-stage extrusion with the use of carbon dioxide as a plasticizer;

Method 7 (Meltrex Method): Meltrex is a modification of the Melting Method. Meltrex is a patented solid dispersion manufacturing process, also based on the melting process. The crucial elements in the Meltrex technology are the use of a special twin screw extruder and the presence of two independent hoppers in which the temperature can vary over a broad temperature range;

Method 8 (Melt agglomeration Method): Melt agglomeration is a modification of the Melting Method. Melt agglomeration allows the preparation of solid dispersions in conventional high shear mixers. It is made by adding the molten “HOST substance” containing the “API” to the heated excipients, by adding the molten “HOST substance” to a heated mixture of “API” and excipients, or by heating a mixture of the “API”, “HOST substance” and excipients to a temperature within or above the melting range of the “HOST substance”. It is also possible to produce stable solid dispersions by melt agglomeration in a rotary processor;

Method 9 (High-Pressure Homogenization): In this method, the suspension of an “API” and “HOST substance” is forced under pressure through a micro- or submicro-sized aperture valve of a high-pressure homogenizer;

Method 10 (Solvent Evaporation Method): In this method dissolved both the “API” and the “HOST substance” in a common solvent and then evaporate the solvent to produce a solid solution. A basic process of preparing or manufacturing solid dispersions of this type consists of dissolving the “API” and the polymeric “HOST substance” in a common solvent or solvents mixture (the list of solvents used is the same as the solvents listed in Method 1). Normally, the resulting films are pulverized and/or milled. The use of the “HOST substances” partially suspended, instead of dissolved, was also can be used. Solvent evaporation procedure usually include vacuum drying, heating of the mixture on a hot plate, slow evaporation of the solvent at low temperature, the use of a rotary evaporator, a stream of nitrogen, or other drying procedure;

Method 11 (Spin-coated films Method): Spin-coated films method is a modification of the Solvent Evaporation Method, which consists of dissolving “API” and “HOST substance” in a common solvent that is dropped onto a clean substrate highly spinned. The solvent is evaporated during spinning;

Method 12 (Spray-drying Method): Spray-drying is a modification of the Solvent Evaporation Method. It consists of dissolving or suspending the “API” and “HOST substance”, then spraying it into a stream of heated airflow to remove the solvent;

Method 13 (Supercritical Fluid (SCF) Process): Supercritical Fluid Process is a modification of the Solvent Evaporation Method. In this method, the “API” particles are solubilized within the SCF (usually carbon dioxide). Several methods of SCF processing can be used to address individual aspects, such as precipitation with a compressed antisolvent process (PCA), solution enhanced dispersion by SCF (SEDS), supercritical antisolvent processes (SAS), the rapid expansion of supercritical solutions (RESS), gas anti-solvent recrystallization (GAS), and aerosol supercritical extraction system (ASES). In this method modification, the technique consists of dissolving the “API” and the “HOST substance” in a common solvent that is introduced into a particle formation vessel through a nozzle, simultaneously with CO2. When the solution is sprayed, the solvent is rapidly extracted by the SCF, resulting in the precipitation of solid dispersion particles on the walls and bottom of the vessel;

Method 14 (Cryogenic Techniques): Cryogenic methods are methods in which a solution of “API” and “HOST substance” in a common solvent is frozen before a low-temperature solvent evaporation procedure. The common solvent can be the same as in the Solvent Evaporation Method. Freezing of the solution can occur after injection. The type of injection device can be a capillary, rotary, pneumatic, ultrasonic nozzle, or other types of the injection device. The location of the nozzle can be above or under the cryogenic liquid level. The composition of cryogenic liquid can be hydrofluoroalkanes, fluorocarbons, N2, Ar, O2, organic solvents, or other types of cryogenic liquid. After cryogenic processing, dry powder can be obtained by various drying processes like spray freeze drying, atmospheric freeze drying, vacuum freeze drying, lyophilization, or other low-temperature drying processes. The list of common solvents used is the same as the solvents listed in Method 1;

Method 15 (Lyophilization/Freeze-Drying Technique): Lyophilization Method is a modification of the Cryogenic Techniques. In this technique, the common solvent from the solution is eliminated through a primary freezing and subsequent drying of the solution containing both “API” and “HOST substance” at reduced pressure. The basic freeze-drying process consists of dissolving the “API” and “HOST substance” in a common solvent, which is immersed in cryogenic liquid until it is fully frozen. Then, the frozen solution is further lyophilized;

Method 16 (Spray Freezing onto Cryogenic Fluids): This Method is a modification of the Cryogenic Techniques. In this technique, the “API” and the “HOST substance” were dissolved in a common solvent and atomized above the surface of a boiling agitated cryogenic liquid refrigerant;

Method 17 (Spray Freezing into Cryogenic Liquids (SFL)): This Method is a modification of the Cryogenic Techniques. It incorporates direct liquid-liquid impingement (by spraying) between the solution and cryogenic liquid to provide intense atomization into microdroplets. The frozen particles are then lyophilized to obtain dry and free-flowing micronized powders.

Method 18 (Spray Freezing into Vapor over Liquid (SFV/L)): This Method is a modification of the Cryogenic Techniques. Freezing of “API” solutions in cryogenic fluid vapors and subsequent removal of frozen solvent. During SFV/L the atomized droplets typically start to freeze in the vapor phase before they contact the cryogenic liquid. As the solvent freezes, the “API” becomes supersaturated in the unfrozen regions of the atomized droplet.

Method 19 (Ultra-Rapid Freezing): This Method is a modification of the Cryogenic Techniques. Application of “API” solution to the solid surface of the cryogenic substrate leads to instantaneous freezing and subsequent lyophilization.

Method 20 (Precipitation Technique, Co-precipitation Method): In the precipitation method the “API” is dissolved in a solvent, which is then added to Non-solvent to precipitate the crystals. Non-solvent is added dropwise to the “API” and “HOST substance” solution, under constant stirring. In the course of the non-solvent addition, the “API” and “HOST substance” are co-precipitated to form micro-particles. In the end, the resulted micro-particle suspension is filtered and dried. The list of solvents used is the same as the solvents listed in Method 1.

Method 21 (Microwave Irradiation Method): This method involves the microwave irradiation reaction between “API” and complexing agent using a microwave oven. The “API” and “HOST substance” in a definite molar ratio are dissolved in a mixture of water and/or organic solvent in a specified proportion into a container. The mixture is reacted in the microwave oven. After the reaction completes, an adequate amount of solvent mixture is added to the above reaction mixture to remove the residual uncomplexed free “API” and “HOST substance”. The precipitate so obtained is separated using a filter, and dried. The list of solvents used is the same as the solvents listed in Method 1.

Method 22 (Energy Input Method): Method comprises the step of exposing a HOST substance and a “API” to an energy input until a bioavailable highly permeable solid dispersion is formed of the “API” as amorphous material entrapped in the HOST substance.

Method 23 (Heat/Shear Energy Input Method): Method comprises the step of exposing a HOST substance and a “API” to an energy input, whereby the energy input is heat and/or shear forces, until a bioavailable highly permeable solid dispersion is formed of the “API” as amorphous material entrapped in the HOST substance.

Method 24 (Combined method): This method includes any combination of Methods 1-23. Methods 1-24 should lead to the formation of the bioavailable solid dispersion.

### III.II. Section

In another aspect provided is a method for the treatment (and for use in manufacturing a pharmaceutical dosage form to treat) of diseases in a human or mammal or animal or bird in need thereof comprising administering a therapeutically effective amount of a compound of FORMULA II (as well as their solutions, gels, colloids, sols, etc.), that is a highly bioavailable highly permeable noncovalent complex, which is obtained by adding solvent to the bioavailable highly permeable solid dispersion of FORMULA I, where the compound of FORMULA I is obtained by co-treating API compound with a HOST substance,
wherein the list of diseases, the list of API compounds, the list of the HOST substances, list of the co-treating methods are the same as in the previous method (the same as in LINK [0061](III.I. Section));
wherein the API compound is selected from GUEST1-9 (the same as API selected in method LINK
(III.I. Section));
wherein the molecular information for the noncovalent complex of FORMULA II is encoded by the formula (the noncovalent complex of FORMULA II is):

[GUEST]k [HOST]n (SOLVENT)m
wherein k is 1 to 100;
wherein m is 1 to 10000;
wherein n is 1 to 10000;
wherein GUEST molecular information is given based on the InChl derived from the InChlKey indicated for each of the API in parentheses after the name of the API compound, in the first position before the sequence numbers (in LINK [0061](III.I. Section)). The molecular information of API compounds can be used on its own as well as on the basis of it (by an skilled in the art) the molecular informations can be obtained for pharmaceutical salts, esters, ethers, free bases and other pharmaceutical salts of these free bases;
wherein HOST molecular information represents the molecular information of a monomer of a polymer; or the smallest representable part of a copolymer chain (defining that copolymer); or molecule (if HOST substance not in polymer form) for compounds that are selected from the HOST substances and can be obtained by an skilled in the art;
wherein SOLVENT molecular information is H2O if solvent is water, but other physiologically acceptable solvents can also be used and the molecular information can be obtained by an skilled in the art;

FIG. 1 shows an example of the chemical formula of one of the noncovalent complexes of FORMULA II based on avermectin Bla and pectin.

### III.III. Section

In another aspect provided is a method for the treatment (and for use in manufacturing a pharmaceutical dosage form to treat) of diseases wherein solid dispersion of FORMULA I is encoded by the formula (the solid dispersion of FORMULA I is):

GUESTk HOSTn
wherein k is 0.0001-0.9, wherein n is 0.1 to 0.9999, where solid dispersions are dispersions in the form of Class C-C, Class C-A, Class A-C, Class A-A, Class M-C, Class M-A solid dispersions containing one, two or more phases (a single phase or a mixture of phases).

In another aspect, other methods are possible for obtaining solid dispersions, non-covalent complexes, complexes with polymers and biopolymers when energy is supplied by any other means, such as (but not limited to) electromagnetic radiation, heat, mechanical action, sound, or in any of the ways described in the scientific and patent literature. As a non-limiting example, an example of exposure by mechanical and acoustic influence is given. Non-covalent complex obtained by the interaction of aqueous solutions of HOST substance and API under conditions of mechano-acoustic and precipitation of the resulting complex with ethyl alcohol. The mechanical-acoustic effect can be carried out in the in a rotary-pulsating apparatus at a rotor speed of 100-10000 rpm.

In another aspect, other various combined methods of obtaining solid dispersions and noncovalent complexes, complexes with polymers and biopolymers described in the patent and scientific literature are possible. As a non-limiting example, a HOST substance is placed in a flask and completely dissolved in ethanol. Then a API is added to the flask. Water is slowly added to the solution and stirred for several hours on a magnetic stirrer at 40-80° C. Then the alcohol part is run under vacuum at a rotary evaporator. The aqueous part is dried by freezing with a lyophilizer.

In another aspect provided is a method for treating (and for use in manufacturing a pharmaceutical dosage form to treat) viral disease wherein solid dispersion, non-covalent complexes, complexes with polymers and biopolymers found in any form described in the scientific and patent literature, and obtained by any of the methods described in the scientific and patent literature are used.

In another aspect, a method of treating (and for use in manufacturing a pharmaceutical dosage form to treat) any viral disease caused by any virus is provided.

In another aspect provided a medical dosage form of FORMULA I of enhanced bioavailability and permeability characterised in that it comprises a highly bioavailable highly permeable solid dispersion or aqueous solutions or suspensions thereof, where the compound of FORMULA I is obtained by co-treating “API” with a HOST substance.
wherein the list of API compounds, the list of the HOST substances, list of the co-treating methods are the same as in the method LINK [0061](III.I. Section).

In another aspect provided a medical dosage form of FORMULA II (as well as their solutions, gels, colloids, sols, etc.), that is a highly bioavailable highly permeable noncovalent complex, which is obtained by adding solvent to the bioavailable highly permeable solid dispersion of FORMULA 1, where the compound of FORMULA I is obtained by co-treating API compound with a HOST substance,
wherein the solvent is usually water and the solvent molecular information is H2O, but other physiologically acceptable solvents or mixtures thereof can also be used;
wherein the list of API compounds, the list of the HOST substances, list of the co-treating methods, the molecular information for the noncovalent complex of FORMULA 11 are the same as in the method LINK [0062](III.II. Section).

In another aspect provided a method for preparing or manufacturing a medical dosage form (and for use it in manufacturing a pharmaceutical dosage form to treat) of FORMULA I of enhanced bioavailability and permeability characterised in that it comprises bioavailable highly permeable solid dispersion or aqueous solutions or suspensions thereof where the compound of FORMULA I is obtained by co-treating “API” with a HOST substance, wherein the list of API compounds, the list of the HOST substances, list of the co-treating methods are the same as in the method LINK [0061](III.I. Section).

In another aspect provided a method for preparing or manufacturing a medical dosage form (and for use it in manufacturing a pharmaceutical dosage form to treat) of FORMULA II (as well as their solutions, gels, colloids, sols, etc.), that is a bioavailable highly permeable noncovalent complex, which is obtained by adding solvent to the bioavailable highly permeable solid dispersion of FORMULA I, where the compound of FORMULA I is obtained by co-treating API compound with a HOST substance,
wherein the solvent is usually water and the solvent molecular information is H2O, but other physiologically acceptable solvents or mixtures thereof can also be used;
wherein the list of API compounds, the list of the HOST substances, list of the co-treating methods, the molecular information for the noncovalent complex of FORMULA II are the same as in the method LINK [0062](III.II. Section).

The medical dosage form may be used for a wide range of low aqueous solubility and dissolution rate active agents or bioactive compounds of the group of ACE inhibitors, adeno-hypophoseal hormones, adrenergic neuron blocking agents, adrenocortical steroids, inhibitors of the biosynthesis of adrenocortical steroids, alpha-adrenergic agonists, alpha-adrenergic antagonists, selective alpha 2-adrenergic agonists, analgesics, antipyretics and anti-inflammatory agents, androgens, anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatelet agents, antiprogestins, antithyroid agents, anti-tussives, antiviral agents, a typical antidepressants, azaspirodecanediones, barbituates, benzodiazepines, benzothiadiazides, beta-adrenergic agonists, beta-adrenergic antagonists, selective beta 1-adrenergic antagonists, selective beta 2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, haematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates, opiod analgesics and antagonists, pancreatic enzymes, pheno thiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic anti-depressants, inhibitors of tubular transport of organic compounds, drugs affecting uterine motility, vasodilators, vitamins and the like, alone or in combination and for use it in manufacturing a pharmaceutical dosage form to treat. Although extensive, this list is not intended to be comprehensive.

### IV. COMBINATION THERAPY

The compositions of the invention are also used in combination with other active ingredients. Non-limiting examples of such other active therapeutic agents are niclosamide, remdesivir, nitazoxanide, chloroquine, lopinavir, ritonavir, hydroxychloroquine and mixtures thereof. The compounds and compositions of the present invention are also intended for use in the general care of patients with diseases, including nutrition, antibiotics (including metronidazole and cephalosporin antibiotics such as ceftriaxone and cefuroxime) and/or antifungal prophylaxis, antipyretics and analgesics, antiemetics (such as metoclopramide) and/or anti-diarrheals vitamin and mineral supplements (including vitamin K and zinc sulfate), anti-inflammatories (such as ibuprofen), pain medications, and medications for other common conditions in the patient population, such as antimalarials (including artemether and artesunate-lumefantrine combination therapy), typhoid (including quinolone antibiotics such as ciprofloxacin, macrolide antibiotics such as azithromycin, cephalosporin antibiotics such as ceftriaxone, or aminopenicillins such as ampicillin) or shigellosis.

It is also possible to combine any compound of the invention with one or more additional active therapeutic agents in a unitary dosage form for simultaneous or sequential administration to a patient. The combination therapy may be administered as simultaneous or sequential administration. In case of sequential administration the combination may be administered in two or more injections.

Co-administration of a compound of the invention with one or more other active therapeutic agents generally refers to the simultaneous or sequential administration of a compound of the invention and one or more other active therapeutic agents such that therapeutically effective amounts of the compound of the invention and one or more other active therapeutic agents are simultaneously present in the patient.

Co-administration comprises administering single doses of the compounds of the invention before or after administering single doses of one or more other active therapeutic agents, for example, administering the compounds of the invention within seconds, minutes or hours after administering one or more other active therapeutic agents. For example, a single dose of the compound of the invention may be administered first, followed by a single dose of one or more other active therapeutic agents within seconds or minutes. Alternatively, a single dose of one or more other therapeutic agents may be administered first, followed by a single dose of the compound of the invention within seconds or minutes. In some cases, it may be desirable to first administer a single dose of the compound of the invention and then, after a few hours (e.g., 1-12 hours), administer a single dose of one or more other active therapeutic agents. In other cases, it may be desirable to first administer a single dose of one or more other active therapeutic agents and then administer a single dose of the compound of the invention a few hours later (e.g., 1-12 hours).

Combination therapy can provide “synergism” and “synergy”, i.e., the effect achieved when the active ingredients are used together is greater than the sum of the effects occurring when the compounds are used separately. Synergistic effects can be achieved when the active ingredients are (1) combined and administered or delivered simultaneously in a combination drug; (2) delivered alternately or in parallel as separate drugs; or (3) by some other scheme. When drugs are alternated, a synergistic effect may be achieved when the compounds are administered or delivered sequentially, for example, in separate tablets, pills, or capsules, or when different injections are given in separate syringes. In general, in alternation, the effective dose of each active ingredient is administered sequentially, that is, serially, whereas in combination therapy the effective doses of two or more active ingredients are administered together. Synergistic antiviral effect means an antiviral effect that exceeds the predicted purely additive effect of the individual compounds of the combination.

A kit comprising a compound of FORMULA I-V is also provided. In some embodiments of the invention, individual kits are provided comprising a compound selected from the group of each of the formulas provided herein and each subgroup and embodiments thereof, including FORMULA II, FORMULA II, FORMULA IV, FORMULA V. In one aspect, the kit includes a compound of FORMULA I. Each of the individual kits described herein may include a label and/or instructions for administering the compound to treat a disease or condition in a subject (e.g., human) in need thereof. In some embodiments of the invention, the disease or condition is a human viral infection. In other embodiments of the invention, each individual kit may also contain instructions for administering additional medical agents in combination with a compound of FORMULA I to treat a disease or condition in a subject (e.g., human) in need thereof. In some embodiments of the invention, the disease or condition is a human viral infection. In each of the embodiments presented herein, there is another embodiment in which the kit includes individual dosage units of the compound described herein. Examples of individual doses may include tablets, tablets, capsules, filled syringes or syringe cartridges, IV bags, etc., each containing a therapeutically effective amount of the compound in question. In some embodiments, the kit may contain a single dosage unit, and in other embodiments, the kit may contain multiple dosage units, such as the number of dosage units required for a particular regimen or period.

In certain embodiments, the product container may be a vial, jar, ampule, preloaded syringe, blister pack, tin, can, bottle, box, or intravenous bag.

Also, certain embodiments of the invention provide for the use of a compound selected from each of FORMULA 1-11 herein in the preparation of a medicament for use in the treatment of diseases in humans.

### IV.I. Section

In another embodiment, the method comprises administering a therapeutically effective amount of a combination pharmaceutical agent (of FORMULA Ill) comprising:
a) a first pharmaceutical composition comprising a compound of FORMULA 1-11 (is 0.01-99.99% of the total weight of the FORMULA Ill), and
b) a second pharmaceutical composition comprising at least one additional therapeutic agent active against infectious viruses or agent that increases the antiviral activity of the FORMULA 1-11 component, or decreases the hepatotoxicity of the FORMULA 1-11 component, or increases the stability of the FORMULA 1-11 component (0.01-99.99% of the total weight of the composition) which may be (but are not limited to):
(Note: The names of internationally registered chemical compounds and pharmaceutical preparations may be given in English. This applies to recognized names such as INN (International Nonproprietary Names)—see WHO International Nonproprietary Names Programme and Classification of Medical Products (https://www.who.int/teams/health-product-and-policy-standards/inn), as well as IUPAC (International Union of Pure and Applied Chemistry) names)

(−)-Carbovir; (−)-Carbovir triphosphate; (−)-Epicatechin; (−)-Epicatechin gallate; (+)-12-Oxocalanolide A; (+)-Catechin Hydrate; (+/−)-trans-U-50488 methanesulfonate salt; (5E)-2-amino-5-(furan-2-ylmethylidene)-1,3-thiazol-4-one; (Melle-4)cyclosporin; (r)-Mandelic acid; (R,R)-asenapine; (S)-(−)-Propranolol hydrochloride; (S)-(+)-rolipram; 1-(Furan-2-ylmethyl)-1,3-diazinane-2,4,6-trione; 1,10-Phenanthroline; 1,10-Phenanthroline-5,6-dione; 1,3,6-Trimethylpyrimido[5,4-e][1,2,4]triazine-5,7-dione; 1,3-Diphenylguanidine; 1,4-Diaminobutane; 1,4-Dicaffeylquinic acid; 1,8-Dihydroxy-4,5-dinitroanthraquinone; 1,9-Pyrazoloanthrone; 10-Hydroxycamptothecin; 1-Amino-8-methoxybenzo[f]quinazoline-7,10-dione; 1-Chloro-2,4-dinitrobenzene; 1-Deoxynojirimycin; 1-Hexadecyl-3-methylimidazolium bromide; 2-(1-Naphthylsulfonyl)-6-aminobenzonitrile; 2-(3,4-Dichlorophenoxy)-5-nitrobenzonitrile; 2-(Morpholinothio)benzothiazole; 2-(Sec-butyldisulfanyl)-1h-imidazole; 2,3,5,6-Tetrakis(furan-2-yl)pyrazine; 2′,3′-Dideoxy-3′-thiacytidine; 2′,3′-Dideoxyadenosine; 2,3-Di-furan-2-yl-quinoxaline-6-carboxylic acid; 2,3-Dihydroxy-5-isopropylcyclohepta-2,4,6-trienone; 2,3-Dihydroxypropyl octanoate; 2,3-Dimercaptosuccinic acid; 2,6,7-Trihydroxy-9-methylxanthen-3-one; 2-[(E)-2-(furan-2-yl)ethenyl]quinoline; 2-[2-(4-Nonylphenoxy)ethoxy]ethanol; 20R-Camptothecin; 2-Aminophenanthrene-9,10-dione; 2be2; 2′-C-methyladenosine; 2′-C-methylcytidine; 2′-C-methylguanosine; 2-Deoxy-2,3-dehydro-n-acetyl-neuraminic acid; 2′-deoxy-2′-fluoro-2′-C-methylcytidine; 2′-Deoxy-5-[(perylen-3-yl)ethynyl]uridine; 2′-Deoxycytidine; 2′-Deoxyguanosine; 2′-Deoxyuridine; 2-Fluoroadenosine; 2-Hydroxy-N′-(2-hydroxybenzoyl)benzohydrazide; 2i0a; 2iOd; 2-Iminobiotin; 2MD; 2-Methoxyestradiol; 2-Methyl-6-(phenylethynyl)pyridine; 2nmw; 2-Phosphoglycolic Acid; 2-Thiophenecarboxylic acid; 3,3′-Diethyl-9-methylthiacarbocyanine iodide; 3,3′-Diindolylmethane; 3,4,5,6-Tetramethylphenanthrene-9,10-dione; 3,6-Acridinediamine, sulfate (2:1); 3,6-Bis(furan-2-yl)-1,2,4,5-tetrazine; 3-Anilino-6-bromo-isocarbostyril; 3′-Azido-2′,3′-dideoxyguanosine; 3-Benzenesulfonyl-5-chloroindole-2-carboxamide; 3gi5; 3-Hydroxybenzoic acid; 3-Me-CycloSal-d4TMP; 4-(4-Toluidino)-1,2-naphthalenedione; 4′,6-Dichloroflavan; 4,7-Epoxyisobenzofuran-1,3-dione, hexahydro-3a,7a-dimethyl-; 4-[(4-Methylbenzyl)amino]phenol; 4-[3-Hydroxyanilino]-6,7-dimethoxyquinazoline; 4-[4-(4-Hydroxyphenyl)hex-3-en-3-yl]phenol; 4-{[2-(Benzyloxy)benzyl]amino}phenol; 4′-Azidocytidine; 4E1RCat; 4′-Ethynyl-2-Fluoro-2′-Deoxyadenosine; 4-Hydroxybenzoic acid; 4-Methoxyphenol; 4-Methyl-DCK; 4-Morpholin-4-ylnaphthalene-1,2-dione; 4-Phenylbutyric acid; 558447-26-0; 5-Bromo-2′-deoxyuridine; 5-Chloro-8-hydroxyquinoline; 6-(4-Tert-butylphenoxy)pyridin-3-amine; 6-(cyclohexylmethoxy)-9H-purin-2-amine; 6,7-Bis(3-hydroxyphenyl)pteridine-2,4-diamine; 6,7-Dimethoxy-2-phenylquinoxaline; 6-Amino-7-bromo-5,8-quinolinedione; 6-Anilino-4-hydroxynaphthalene-2-sulphonic acid; 6-Azauridine; 6-Benzyl-1-benzyloxymethyl-5-isopropyl uracil; 6-Hydroxydopamine hydrobromide; 6-Methyl-2-(phenylazo)-3-pyridinol; 7-[6-(Benzimidazol-1-yl)hexoxy]chromen-2-one; 7-Amino-6-methoxy-2-methyl-5,8-quinolinedione; 7-Aminocephalosporanic acid; 7-Deaza-2′-C-acetylene-adenosine; 7-Deaza-2′-C-methyladenosine; 7-Ethyl-10-hydroxycamptothecin; 8-Aminoquinoline-5,6-dione; 8-Hydroxy-2-methylquinazolin-4(3H)-one; 8-Hydroxyquinoline; 8-OH-Dpat; 9,10-Phenanthrenequinone; 9-Aminoacridine; 9-Aminocamptothecin; 9VR1J6U4XG; A43D; A-790742; A-837093; Abacavir; Abemaciclib; Abexinostat; ABT-072; Abt-737; Abt-751; Acelarin; Acenocoumarol; Acetazolamide; Acetophenazine maleate; Acetylcholine; Acetylcysteine; Acetylpepstatin; Acetylshikonin; Acriflavine; Acrisorcin; Acteoside; Acyclovir; Adafosbuvir; Adam ii; Adavosertib; Adefovir; Adefovir dipivoxil; Adenine; Adenosine; Adenosine 5′-monophosphate monohydrate; Adenosine monophosphate; Adenosine-5′-diphosphate; Adenosine-5′-triphosphate; Adiphenine hydrochloride; Adipic acid; Afegostat; Afimoxifene; AG14361; Albendazole; Albendazole oxide; Aleglitazar; Alendronic acid; Alfuzosin; Alfuzosin hydrochloride; Alibendol; Alisertib; Aliskiren; Alisporivir; Allethrin; Allitinib; Alosetron hydrochloride; Alovudine; Alpha-D-Galactopyranose; alpha-D-mannose 6-phosphate; alpha-Hederin; alpha-Ketoamide inhibitor 11r; alpha-L-arabinopyranose; alpha-Maltotriose; alpha-Mangostin; Alprenolol; Alprenolol hydrochloride; Alprostadil; Alsterpaullone; Altrenogest; Altretamine; Alverine; Alverine citrate; Alvespimycin; Alvocidib; Amantadine; Amantadine hydrochloride; Amaranth; AMD 3465; Amdoxovir; AMG-131; AMG-232; AMG-900; Amiloride; Aminocaproic acid; Amiodarone; Amiodarone hydrochloride; Amitifadine; Amitriptyline; Amitriptyline hydrochloride; Amlexanox; Amlodipine; Amlodipine besylate; Amodiaquin dihydrochloride dihydrate; Amodiaquine; Amonafide; Amorolfine; Amoxapine; Amoxicillin; Amphotericin b; Ampicillin; Amprenavir; Amrubicin; Amsacrine; Amsacrine hydrochloride; Amuvatinib; Anacetrapib; Anagrelide; Ancitabine hydrochloride; Andarine; Andrographolide; Androstanolone; Androsterone; Anethole trithione; Angustibalin; Anidulafungin; Anileridine; Anisomycin; Antazoline hydrochloride; Anthralin; antimycin A1; Antipyrine; Apalutamide; Apatinib; Apigenin; Apilimod; Apitolisib; Apixaban; Aplaviroc; Aplaviroc hydrochloride; Apomorphine; Aprepitant; Apricitabine; Arachidonic acid; Arbidol; Arctigenin; Aripiprazole; Artemether; Artemisinin; Artesunic acid; Aspartame; Aspirin; Astemizole; Asterric acid; ASTX660; Asunaprevir; AT9283; Ataciguat; Ataluren; Atazanavir; Atevirdine; Atipamezole; Atomoxetine; Atomoxetine hydrochloride; Atopaxar; Atorvastatin; Atrasentan; Aurintricarboxylic acid; AV-412 free base; Avasimibe; AVN-944; AVP-13358; Axitinib; AZ-960; Azacitidine; Azaribine; Azatadine; Azathioprine; Azd4547; AZD8330; Azelaic acid; Azelastine; Azelastine hydrochloride; Azelnidipine; Azithromycin; Azoxystrobin; Azvudine; B5R51Q1D64; Baicalein; Baicalin; Bakuchiol; Balofloxacin; Baloxavir; Baloxavir marboxil; Bardoxolone methyl; Baricitinib; Batimastat; Bay 41-2272; BAY 41-4109; Bazedoxifene; Bazedoxifene acetate; Bdcrb; Beclabuvir; Beclomethasone; Belinostat; Belladonna total alkaloids; Benazepril hydrochloride; Bendamustine; Benidipine hydrochloride; Benperidol; Benserazide; Benserazide hydrochloride; Bentamapimod; Benzamidine; Benzbromarone; Benzenesulfonamide; Benzethonium chloride; Benzethonium; Benziodarone; Benzoic acid; Benzophenone; Benztropine; Benztropine mesylate; Benzydamine; Benzydamine hydrochloride; Benzyl 4-hydroxybenzoate; Benzyl benzoate; Benzyldimethyl(tridecyl)ammonium chloride; Bepridil; Berbamine; Berberine chloride; Berberine; Bersacapavir; Berzosertib; Besifovir; Beta-Carotene; beta-CYCLODEXTRIN; beta-D-Ribopyranose; beta-Escin; beta-Lactose; beta-Lapachone; beta-L-Arabinose; Betaxolol; Betazole hydrochloride; Betulin; Betulinic acid; Bevirimat; Bexarotene; BGT-226 free base; BI 2536; Bictegravir; Bifemelane; Bifonazole; BILR-355; Binodrenal; Biotin; Biperiden; Biperiden hydrochloride; Birinapant; Bis(2-ethylhexyl) phthalate; Bisbenzimide; Bishelenalinyl malonate; Bisibutiamine; Bisoprolol fumarate; Bisphenol a; Bisphenol a diglycidyl ether; Bithionol; Bitolterol; Blonanserin; Boceprevir; Boldine; Bortezomib; Boscalid; Bosutinib; Brecanavir; Brefeldin a; Brequinar; Brilliant green; Brincidofovir; Brinzolamide; Brivanib; Brivanib alaninate; Brivudine; Bromazine; Bromfenac; Bromhexine hydrochloride; Bromocriptine; Bromperidol; Brompheniramine maleate; Bucindolol; Budesonide; Bumetanide; Bunamidine hydrochloride; Buparlisib; Buparvaquone; Bupranolol; Bupropion hydrochloride; Buspirone; Butacaine; Butenafine; Butoconazole; Butoconazole nitrate; Butriptyline; BX-912; C.I. Direct blue 53; C188-9; Cabergoline; Cabotegravir; Cabozantinib; Caffeic acid; Caffeine; Calanolide; Calanolide a; Calcimycin; Calcitriol; Calpeptin; Cambridge id 6798241; Camostat; Camptothecin; Camylofin; Candesartan cilexetil; Canertinib; Cannabinol; Canrenone; Capivasertib; Capravirine; Capsaicin; Captopril; Carbamazepine; Carbamylcholine; Carbetapentane; Carbetapentane citrate; Carbinoxamine maleate; Carbodine; Carboxyamidotriazole; carboxy-N,N,N-trimethylmethanaminium; Carfilzomib; Carmofur; Carphenazine dimaleate; Carprofen; Carvedilol; Caspofungin; Cbr15S3hnj; Cbz-lNal-Leu-Ala((S)-2-oxopyrrolidin-3-yl)-al; Cbz-Ala-Val-Phe-ol; CC-401; CCT129202; Cediranib; Cefaclor; Cefazolin sodium; Cefixime; Cefotaxime sodium; Cefotiam hydrochloride; Cefoxitin sodium; Cefuroxime sodium; Celastrol; Celecoxib; Celgosivir; Cenicriviroc; Cenicriviroc mesylate; Censavudine; Cephalothin; Cephapirin sodium; Cepharanthine; Ceritinib; Cetalkonium chloride; Cetrimide; Cetrimonium bromide; Cetylpyridinium bromide; Cetylpyridinium chloride; Cetylpyridinium chloride monohydrate; Cetylpyridinium; Chelerythrine chloride; Chelerythrine; Chicoric acid; Chidamide; Chikv-IN-2; CHIR-124; Chlorambucil; Chlorcyclizine; Chlorcyclizine hydrochloride; Chlordiazepoxide; Chlorhexidine hcl; Chlormadinone acetate; Chlorogenic acid; Chloroquine; Chloroquine monophosphate; Chloroquine phosphate; Chloroxine; Chlorpheniramine maleate; Chlorpromazine; Chlorpromazine hydrochloride; Chlorprothixen; Chlorprothixene; Chlorquinaldol; Chlortetracycline; Chlortetracycline hcl; Chlortetracycline hydrochloride; Cholesterol; Choline; Cianidanol; Ciclesonide; Ciclopirox; Ciclopirox olamine; Ciclosporin; Cidofovir; Cilnidipine; Ciluprevir; Cinacalcet hydrochloride; Cinnarizine; Ciprofloxacin hydrochloride; Cisapride; Cisapride; Citalopram; Citalopram hydrobromide; Citric acid; Cladribine; Clemastine; Clemastine fumarate; Clemizole; Clevudine; Clevudine; Climbazole; Clinofibrate; Clobetasol propionate; Clobetasone butyrate; Clofarabine; Clofazimine; Clofenotane; Clofoctol; Clomifene; Clomiphene; Clomiphene citrate; Clomipramine; Clomipramine hydrochloride; Cloperastine hydrochloride; Clorgyline hydrochloride; Clorsulon; Closantel; Clothiapine; Clotrimazole; Clozapine; Cndac; Codeine hydrochloride; Colchicine; Colistinmethanesulfonic acid; Combivir; Combretastatin A4; Congo red; Conjuncain; Copanlisib; Cordycepin; Costatolide; Cozaar; CP-673451; CP-91149; Crenolanib; Cridanimod; CTS-1027; CU-3; Cudc-101; CUDC-427; Curcumin; Cvt-313; CX-5461; Cyclizine; Cyclizine dihydrochloride; Cyclobenzaprine; Cyclobenzaprine hydrochloride; Cycloguanil; Cycloguanil hydrochloride; Cycloheximide; Cyclophosphamide; CycloSal-d4TMP; Cycloserine; Cydectin; Cynarine; Cyproheptadine; Cyproheptadine hydrochloride; Cyproterone acetate; Cysteamine hydrochloride; Cysteine; Cytarabine; Cytarabine hydrochloride; Cytisine; Dabigatran etexilate mesylate; Dabrafenib; Dacarbazine; Daclatasvir; Dactinomycin; Dactolisib; Daidzein; Danazol; Daniquidone; Danoprevir; Danusertib; Daphnetin; Dapivirine; Daporinad; Dapoxetine; Darifenacin hydrobromide; Darunavir; Dasabuvir; Dasatinib; Daunorubicin; Daunorubicin hydrochloride; D-Cellobiose; Decitabine; Deferasirox; Deferoxamine mesylate; Delanzomib; Delapril hydrochloride; Delavirdine; Delavirdine mesylate; Deleobuvir; Demeclocycline; Demeclocycline hydrochloride; Deoxycholic acid; Dequalinium chloride; Dequalinium; Deracoxib; Derenofylline; Desacetylvinblastine amide; Deserpidine; Desipramine; Desipramine hydrochloride; Deslanoside; Desloratadine; Dexamethasone; Dexamethasone isonicotinate; Dexbrompheniramine maleate; Dexchlorpheniramine maleate; Dexelvucitabine; Dexnorgestrel acetime; D-Galactose; DHAD; Diacerein; Diazepam; Diazoxide; Dibenzepin; Dibenzepin hydrochloride; Dibucaine; Dichlorophen; Dichlorphenamide; Diclazuril; Diclofenac; Diclofenac sodium; Dicloxacillin sodium monohydrate; Dicumarol; Dicyclomine; Dicyclomine hydrochloride; Didanosine; Didehydrothymidine-cyclic-benzophosphate, 3,5-D; Dienestrol; Diethylstilbestrol; Diflunisal; Digitoxin; Digoxigenin; Digoxin; Dihydroergocristine; Dihydroergocristine mesylate; Dihydroergotamine; Dihydrotachysterol; Dihydroxypyrimidine carboxamide, 3; Diltiazem; Diltiazem hydrochloride; Dimethyldioctylammonium chloride; Dinaciclib; Diosmetin; Dioxybenzone; Dipentamethylenethiuram disulfide; Diperodon; Diperodon hydrochloride; Diphenhydramine; Diphenidol; Diphenylpyraline hydrochloride; Dipyridamole; Dipyridodiazepinone deriv. 42; Dipyrithione; Disodium hydrogen phosphate; Disulfiram; Dizan; Dizocilpine maleate; Dmipopmt; Dmp-323; Docetaxel; Docosahexaenoic acid; Dofetilide; Dolutegravir; Dolutegravir sodium; Domiphen bromide; Domperidone; Donepezil hydrochloride; Dopamine hydrochloride; Dopexamine; Doramapimod; Doramectin; Doravirine; Doripenem; Dorzolamide; Dovitinib; Doxazosin; Doxazosin mesylate; Doxepin hydrochloride; Doxercalciferol (hectorol); Doxorubicin; Doxorubicin hydrochloride; Doxycycline hydrochloride; Doxylamine succinate; DPC-082; DPC-961; Drofenine hydrochloride; Droloxifene; Drometrizole; Dronabinol; Dronedarone; Dronedarone hydrochloride; Droperidol; Drospirenone; Drostanolone propionate; D-serine; d-Tartaric acid; Duloxetine; Duloxetine hydrochloride; Dyclonine hydrochloride; Dydrogesterone; Dynamax; E100; Ebastine; Ebselen; Econazole nitrate; Ecopipam; Edaravone; Edoxudine; Efavirenz; Elacestrant; Elacridar; Elaidic acid; Elbasvir; Elesclomol; Eletriptan; Eliprodil; Ellagic acid; Ellipticine; Eltrombopag; Elvitegravir; Elvucitabine; Embelin; Emedastine difumarate; Emivirine; Emodin; Emricasan; Emtricitabine; Enclomiphene citrate; Endoxifen; Endurobol; Enfuvirtide; Enobosarm; Enoximone; Enoxolone; Ensulizole; Ensovibep; Entecavir; Entecavir triphosphate; Entinostat; Enviroxime; Enzastaurin; Eperisone hydrochloride; Ephedrine sulfate; Epigallocatechin; Epigallocatechin gallate; Epinephrine; Epinephrine hydrogen tartrate; Epirubicin; Epirubicin hydrochloride; Eprazinone dihydrochloride; Ergocalciferol; Erlotinib; Erteberel; Erythromycin; Erythromycin estolate; Erythromycin propionate; Escitalopram oxalate; Esculetin; Esmolol hydrochloride; Estradiol; Estradiol acetate; Estradiol cypionate; Estragole; Estramustine; Estriol; Estrone; Ethacridine lactate monohydrate; Ethacrynic acid; Ethanol; Ethinyl estradiol; Ethopabate; Ethopropazine hydrochloride; Ethoxyquin; Ethoxzolamide; Ethyl gallate; Ethyl nitrite; Ethylestrenol; Etofenamate; Etoposide; Etravirine; Etravirine impurity 5; Evans blue; Everolimus; Exaluren; Excenel; Exemestane; Exifone; Ezetimibe; F2-NH-DABO 9d; Faldaprevir; Famciclovir; Farglitazar; Fasudil; Favipiravir; Febuxostat; Felodipine; Fenbendazole; Fenbufen; Fenobam anhydrous; Fenoldopam; Fenoldopam mesylate; Fenoverine; Fenretinide; Fiacitabine; Fialuridine; Fiboflapon; Fidaxomicin; Filgotinib; Filibuvir; Filociclovir; Fimepinostat; Fingolimod; Fipexide hydrochloride; Firsocostat; Fisetin; Flavin mononucleotide; Flavone; Flavopiridol hydrochloride; Flecainide; Flecainide acetate; Floxuridine; Fludarabine; Flufenamic acid; Flumequine; Flunarizine; Flunarizine hydrochloride; Fluorescein; Fluorouracil; Fluoxetine; Fluoxetine hydrochloride; Flupentixol; Flupentixol dihydrochloride; Fluphenazine; Fluphenazine hydrochloride; Flupirtine; Flupirtine maleate; Flurazepam; Flurbiprofen; Fluspirilene; Flutamide; Fluticasone propionate; Fluvastatin; Fluvastatin sodium; Fluvoxamine; Fluvoxamine maleate; Folic acid; Foretinib; Formononetin; Forodesine; Forskolin; Fosamax; Foscarnet; Foscarnet sodium; Fostamatinib; Fulvestrant; Furosemide; Furoxone; Fusidate sodium; Fusidic acid; Galantamine; Galidesivir; Gallic acid; Galloflavin; Gallotannin; Gamma-Aminobutyric Acid; gamma-Oryzanol; Ganciclovir; Ganetespib; Gatifloxacin; GC376 sodium; Gdc-0152; Gefitinib; Geldanamycin; Gemcitabine; Gemcitabine hydrochloride; Genistein; Gentian violet; Gentian violet cation; Gestrinone; Gilteritinib; Gimatecan; Givinostat hydrochloride monohydrate; Glafenine hydrochloride; Glecaprevir; Glesatinib; Gliclazide; Glipizide; Gliquidone; Glutamic acid; Glutamine; Glutaric acid; Glutathione; Glyburide; Glycine, N-[imino(phosphonoamino)methyl]-N-methyl-; Glycopyrrolate; Glycyrrhizic acid; GNF-Pf-159; GNF-Pf-2335; GNF-Pf-4300; GNF-Pf-888; Go-6976; Golvatinib; Gossypol; Gossypol acetic acid; Gossypol hexaacetate; Granisetron; Grazoprevir; Guaiazulene; Guanabenz acetate; Guanidino-Oseltamivir Carboxylicacid; Guanidino-Tamiphosphor; Guanosine; GW0742; GW4511; GW678248; H-Ala-Lys-Val-Thr-Met-Thr-Cys-Ser-Ala-Ser-OH; Halcinonide; Halofantrine; Halofantrine hydrochloride; Haloperidol; Haloxon; Harmaline; Harmine; Harmine hydrochloride; Harringtonine; HBF-0259; HDP-HPMPA; Helioxanthin 8-1; Hematoxylin; Hesperetin; Hexachlorophene; Hexestrol; Hexylcaine hydrochloride; Hexylresorcinol; Histamine; Histidine; HIV-1 inhibitor-43; Homidium bromide; Honokiol; Hpmpa; Huperzine a; Hydergine; Hydrocortisone; Hydrocortisone acetate; Hydroquinone; Hydroxychloroquine; Hydroxychloroquine sulfate; Hydroxyflutamide; Hydroxyprogesterone; Hydroxyprogesterone caproate; Hydroxytyrosol; Hydroxyurea; Hydroxyzine; Hydroxyzine pamoate; Hyoscyamine; Hypericin; Hyperoside; Hypoxanthine; Ibrutinib; Ibufenac; Ibuprofen; IC9564; Icaritin; ICG-001; Icosapent; Idarubicin; Idarubicin hydrochloride; Idasanutlin; Idazoxan; Idelalisib; Idoxuridine; Idronoxil; Ifenprodil; Iloperidone; Imatinib; Imatinib mesylate; Imidocarb; Iminostilbene; Imipenem; Imipramine; Imipramine hydrochloride; Imiquimod; Imperatorin; Inarigivir soproxil; INCB-9471; Indibulin; Indinavir; Indinavir sulfate; Indiplon; Indirubin; Indobufen; Indocyanine green; Indole-3-carbinol; Indomethacin; Indoprofen; Indoramin hydrochloride; Ingenol mebutate; Inosine; Inositol; Invokana; INX-08189; lodoacetamide; lodoquinol; lofetamine; iOWH-032; Ipatasertib; Ipriflavone; Irbesartan; Irinotecan; Irinotecan hydrochloride; Irosustat; Isoconazole; Isocycloheximide; Isoliquiritigenin; Isoorientin; Isopropamide; Isopropyl unoprostone; Isoquercetin; Isoreserpin; Isosulfan blue; Isothymonin; Isotretinoin; Isradipine; Istodax; Istradefylline; ITD-1; Itraconazole; Itraconazolum [latin]; Ivacaftor; Ivermectin; Ixazomib; JE-2147; JNJ-7706621; Kaempferol; Kalafungin; Kenpaullone; Ketanserin; Ketoconazole; Ketorolac; Ketorolac tromethamine; Ketotifen; Ketotifen fumarate; Kinetin; KNI-1931; Ksc-16-200S2C; Ku-0063794; Kynostatin 272; L-685434; Labetalol; Lamivudine; Lanatoside c; Laninamivir; Laninamivir octanoate; Lansoprazole; Lapatinib; Laropiprant; Lasalocid sodium; LCL161; Ledipasvir; Leflunomide; Lenvatinib; LEO 275; Lercanidipine; Lercanidipine hydrochloride; Lersivirine; Lestaurtinib; Letermovir; Leucine; Leucomethylene blue; Levallorphan; Levobupivacaine hydrochloride; Levodopa; Levofloxacin; Levomethadyl acetate hydrochloride; Levonordefrin; Levosimendan; Levothyroxine; Lexibulin; L-Glutamic acid hydrochloride; Licofelone; Lidoflazine; Lificiguat; Ligustrinin b; Linoleic acid; Linsitinib; Liothyronine; Liothyronine sodium; Liquiritigenin; Liranaftate; Lithocholic acid; Lobeline; Lodosyn; Lofepramine hydrochloride; Lofexidine; Lomefloxacin; Lomefloxacin hydrochloride; Lomerizine hydrochloride; Lomibuvir; Lonafarnib; Loperamide; Loperamide hydrochloride; Lopinavir; Loratadine; Lorcaserin; Lorlatinib; L-ornithine; Lornoxicam; Lovastatin; Loviride; Loxapine; Loxapine succinate; L-Tartaric acid; Lucanthone hydrochloride; Luliconazole; Lumacaftor; Lumefantrine; Lumicitabine; Luminespib; Lusutrombopag; Luteolin; Lynestrenol; Lysergide; Lysine; Magnolol; Malachite green oxalate; Maleic acid; Malonic acid; Malonoben; Malotilate; Manidipine; Manidipine dihydrochloride; Maprotiline; Maprotiline hydrochloride; Maraviroc; Mardepodect; Maribavir; Masoprocol; Matrine; Maxacalcitol; MC-1220; Mebendazole; Mebeverine hydrochloride; Mecamylamine; Mechlorethamine hydrochloride; Meclizine hydrochloride; Meclocycline sulfosalicylate; Medphalan; Medroxyprogesterone; Mefenamic acid; Mefloquine; Mefloquine hydrochloride; Melitracen hydrochloride; Melittin; Melphalan; Memantine; Memantine hydrochloride; Menadione; Menadione sodium bisulfite; Meptazinol hydrochloride; Mepyramine; Mequitazine; Merbromin; Mercaptopurine; Mericitabine; Merimepodib; Mesalamine; Mesoridazine; Mestranol; Met kinase inhibitor; Metarrestin; Metergoline; Methacycline; Methacycline hydrochloride; Methadone hydrochloride; Methapyrilene hydrochloride; Methazolamide; Methdilazine hydrochloride; Methionine; Methixene hydrochloride; Methotrexate; Methyl methanesulfonate; methyl-2-pyrrolidinone, 17b; Methyldopa; Methyldopa sesquihydrate; Methylene blue; Methylene blue cation; Methylergonovine; Methylprednisolone; Methysergide; Methysergide maleate; Metildigoxin; Metixene hydrochloride; Metolazone; Metoprine; Mevastatin; Mianserin; Mianserin hydrochloride; Mibefradil; Mibefradil dihydrochloride; Michellamine b; Miconazole; Miconazole nitrate; Midostaurin; Mifepristone; Milciclib; Miltefosine; Minocycline hydrochloride; Minoxidil; Mitomycin c; Mitotane; Mitoxantrone; Mitoxantrone hydrochloride; Mizolastine; MK-1107; MK-2206; MK-3281; MK-4965; MK-5108; MK-6186; MK-7445; MK-8245; MK-8325 free base; MK-944a; ML322; ML355; MLN8054; Mocetinostat; Mocravimod; Momelotinib; Mometasone furoate; Monobenzone; Montelukast sodium; Moricizine; Morin; Morphine sulfate; Morphothiadin; Mosapride citrate; Motesanib; Moxalactam disodium; Moxaverine; Moxifloxacin hydrochloride; Mozenavir; Mthpc; Mupirocin; Mycophenolate mofetil; Mycophenolic acid; Myricetin; N-(1,3-benzothiazol-2-yl)-2-methoxybenzamide; N-(1,4-dioxonaphthalen-2-yl)benzamide; N-(1H-benzimidazol-2-yl)-2-fluorobenzamide; N-(1H-benzimidazol-2-yl)-2-methoxybenzamide; N-(2,6-Dichlorobenzylidene)-N′-amidinohydrazine; N-9-Methoxynonyldeoxynojirimycin hydrochloride; Naftifine hydrochloride; Naftopidil; Naloxone; Naltrexone; Nanaomycin; Naphazoline hydrochloride; Naproxen; Naratriptan; Naringenin; Narlaprevir; Navelbine; Navitoclax; NBD-14270; NCGC00090797-11; NCGC00179454-04; Nebivolol hcl; Nefazodone; Nefazodone hydrochloride; Nefopam; Nefopam hydrochloride; Nelfinavir; Nelfinavir mesylate; Neomycin; Nesbuvir; N-ethylmaleimide; Netilmicin; Nevirapine; Nialamide; Nicardipine; Nicardipine hydrochloride; Nicergoline; Nickeldibutyldithiocarbamate; Niclosamide; Nicotine; Nifedipine; Niflumic acid; Nifuroxazide; Nimesulide; Nimodipine; Nimustine hydrochloride; Nintedanib; Niraparib; Nisoldipine; N-Isopropyl-N′-phenyl-p-phenylenediamine; Nitazoxanide; Nitrendipine; Nitrofurantoin; Nitrofurazone; Nitroxoline; N-Methylpyrimidone, 17; Nocodazole; Nolvasan; Nomifensine maleate; Norcantharidin; Nordihydroguaiaretic acid; Norepinephrine; Norethisterone enanthate; Norfloxacin; Normethadone; North-methanocarbathymidine; Nortriptyline; Nortriptyline hydrochloride; Novobiocin; N-phenylanthranilic acid; NSC158393; NSC351815; NSC640065; NSC651084; NSC666863; NSC697445; N-tert-butyl isoquine; Nucleozin; Nylidrin; Nystatin (fungicidin); Nystatin A1; o6-Benzylguanine; Octocrylene; Odanacatib; Ofloxacin; Olanzapine; Oleanolic acid; Oleic acid; Olprinone; Omacetaxine mepesuccinate; Omadacycline; Ombitasvir; Omeprazole; Omipalisib; Onalespib; Oncovin; Ondansetron; ONO-5334; Opipramol; Opreal_836388; Oprozomib; Orphenadrine; Orphenadrine hydrochloride; Oseltamivir; Oseltamivir acid; Oseltamivir phosphate; Osi-027; Osi-930; Osimertinib; Ospemifene; Osthole; Otamixaban; Otilonium bromide; Otilonium; Ouabain; Ouabain octahydrate; Oxalacetic acid; Oxalic acid; Oxatomide; Oxethazaine; Oxfendazole; Oxibendazole; Oxiconazole nitrate; Oxprenolol hydrochloride; Oxyclozanide; Oxymatrine; Oxymetazoline; Oxymetazoline hydrochloride; Oxymetholone; Oxyphenbutazone; Oxyphencyclimine hydrochloride; Oxytetracycline; Oxytetracycline dihydrate; Oxytetracycline hydrochloride; Paclitaxel; Paclitaxel (taxol); Palbociclib; Palinavir; Palmitic acid; Palonosetron hydrochloride; Pamicogrel; Panobinostat; Papaverine; Papaverine hydrochloride; Paritaprevir; Paritaprevir dihydrate; Paroxetine; Paroxetine hydrochloride; Parthenolide; Parthenolide; Parthenolide; Pasiniazid; Paxlovid; Pazopanib; Pazopanib hydrochloride; PD0166285; PDK1 inhibitor AR-12; Pecazine; Pectamol; Pemetrexed; Penbutolol sulfate; Penciclovir; Penfluridol; Pentamidine; Pentoxifylline; Pepstatin; Peramivir; Perazine; Pergolide mesylate; Perhexiline; Perhexiline maleate; Periciazine; Perospirone hydrochloride; Perphenazine; PF-00562271 free base; PF-03882845; PF-04217903; PF-04691502; PF-3758309; PF-46396; PF-477736; pf74; PH-797804; Pha-665752; PHA-793887; Phenazopyridine; Phenazopyridine hydrochloride; Phenelzine; Phenformin hydrochloride; Phenol; Phenoxybenzamine; Phenprocoumon; Phentolamine; Phentolamine mesylate; Phenylalanine; Phenylbutazone; Phorbol 12-myristate 13-acetate; Phosphonatoformate; Phosphoric acid; Phthalic acid; Physostigmine; Phytic acid; PI-103; Piboserod; Pibrentasvir; Piceid; Picropodophyllin; Pictilisib; Pifoxime; Pilaralisib; Pimethixene; Pimethixene maleate; Pimobendan; Pimodivir; Pimozide; Pinaverium bromide; Pindolol; Piperacetazine; Piperidolate hydrochloride; Pirarubicin; Pirfenidone; Piritrexim; Pirlindole mesylate; Pirodavir; Pivampicillin; Pivmecillinam hydrochloride; Pizotifen; Pizotifen malate; PL-100; Platinum, diamminedichloro-; Pleconaril; Plerixafor; Plerixafor octahydrochloride; Plumbagin; PLX-4720; Pnu-103017; Pocapavir; Podofilox; Ponatinib; Posaconazole; Pradigastat; Pradimicin a; Pradimicin s; Pralatrexate; Pramipexole; Pramoxine hydrochloride; Pranlukast; Praziquantel; Prazosin; Prazosin hydrochloride; Precose; Prednisolone tebutate; Prednisone; Preladenant; Prenylamine lactate; Presatovir; Prima-1met; Primaquine; Primaquine phosphate; Prinaberel; Proadifen hydrochloride; Procainamide hydrochloride; Prochlorperazine; Prochlorperazine edisylate; Prochlorperazine maleate; Procyclidine hydrochloride; Proflavine; Progesterone; Proguanil hydrochloride; Promazine; Promazine hydrochloride; Promethazine; Promethazine hydrochloride; Pronetalol; Propafenone; Propafenone hydrochloride; Proparacaine hydrochloride; Propidium iodide; Propoxyphene hydrochloride; Propranolol hydrochloride; Propyl gallate; Proscillaridin; Prostratin; Protionamide; Protoporphyrin ix; Protriptyline; Proxalutamide; psi 7851; psi-352938; Pterostilbene; PU-H71; Puromycin dihydrochloride; Purpurogalline; Pyrantel pamoate; Pyrazinamide; Pyridoxal; Pyridoxine; Pyrilamine maleate; Pyrimethamine; Pyrithione; Pyronaridine; Pyrophosphoric acid; Pyroxamide; Pyrrollo-A; Pyruvate; Pyrvinium pamoate; Q9LK8R766M; Quercetin; Quercetin dihydrate; Quercitrin; Quinacrine; Quinacrine dihydrochloride; Quinacrine dihydrochloride dihydrate; Quinestrol; Quinidine; Quinidine hydrochloride monohydrate; Quinine; Quinine sulfate dihydrate; Quisinostat; R-82913; Rabeprazole sodium; Rabusertib; Racecadotril; Radalbuvir; RAF265; Rafoxanide; Ralimetinib; Raloxifene; Raloxifene hydrochloride; Raltegravir; Raltegravir potassium; Raltitrexed; Raluridine; Ramosetron hydrochloride; Rapacuronium bromide; Ravidasvir; Rebastinib; Reboxetine mesylate; Regorafenib; Remdesivir; Rescinnamine; Reserpine; Resiniferatoxin; Resiquimod; Resorcinol; Resveratrol; Retigabine; Retinol; Riamilovir sodium dihydrate; Ribavirin; Riboflavin; Ricolinostat; Rifabutin; Rilematovir; Rilmenidine; Rilpivirine; Riluzole; Rimantadine; Rimantadine hydrochloride; Rimonabant; Risperidone; Rita; Ritanserin; Ritonavir; Rivaroxaban; Riviciclib; R04929097; Rofecoxib; Rolipram; Rolitetracycline; Rosmarinic acid; Rotenone; Rotigotine; Rottlerin; Roxadustat; RSV604; Ruboxistaurin; Rubschisandrin; Rucaparib; Rupintrivir; Rutaecarpine; Rutin; Ruxolitinib; Saccharin; S-Adenosyl-DL-methionine; Safinamide; Salicylic acid; Salinomycin; Salinomycin sodium; Salmeterol; Salmeterol xinafoate; Sancycline; Sanguinarine; Sanguinarium chloride; Sapanisertib; Sapitinib; Saponaretin; homovitexin; Saquinavir; Saquinavir mesylate; Saracatinib; Sarafloxacin; Sarecycline; Sarpogrelate; Sarpogrelate hydrochloride; SB-505124; SB-743921; Sch 58261; Scy-635; SD-06; sd146; Sebacic acid; Secnidazole; Secoisolariciresinol; Securinine; Selexipag; Seliciclib; Semaxanib; Sennoside a, analytical standard; Sensit; Sepantronium; Serdemetan; Serotonin hydrochloride; Sertaconazole nitrate; Sertindole; Sertraline; Sertraline hydrochloride; SGI-1776; Sgx-523; Sibutramine; Silibinin; Silmitasertib; Silymarin; Simeprevir; Simvastatin; Sinefungin; Sinequan; Sinomenine; Sirolimus; Sisunatovir; Sitagliptin; SNS-032; SNX-2112; Snx-5422; Sodium hydroxide; Sodium hypochlorite; Sofalcone; Sofosbuvir; Solifenacin hydrochloride; Sophoridine; Sorafenib; Sorafenib tosylate; Sorivudine; Sotradecol; Sovaprevir; Spironolactone; Staurosporine; Stavudine; Stavudine triphosphate; Stearic acid; Streptomycin; Stubomycin; SU9516; Succinic acid; Succinylcholine; Sufentanil citrate; Sulbactam; Sulconazole nitrate; Sulfadiazine; Sulfamethazine sodium; Sulfamethoxazole; Sulfapyridine; Sulfasalazine; Sulindac sulfone; Sultiame; Sumatriptan; Sunitinib; Sunitinib malate; Suprofen; Suramin; Suramin (sodium salt); Swertiamarin; Tacrine; Tacrine hydrochloride monohydrate; Tak-285; Tak-715; TAK-733; TAK-901; Talinolol; Talmapimod; Talviraline; Tame; Tamibarotene; Tamiphosphor; Tamoxifen; Tamoxifen citrate; Tandutinib; Tanespimycin; Tangeretin; Tanshinone i; Tanshinone iia; Tanzisertib; Tariquidar; Tauroursodeoxycholic acid; Taxifolin; Tazarotene; Tazobactam sodium; TCRB; Tecovirimat; Tegaserod; Tegaserod maleate; Tegobuvir; Telaprevir; Telbivudine; Telinavir; Telmisartan; Telotristat; Telotristat ethyl; Temoporfin; Temsavir; Tenofovir; Tenofovir alafenamide; Tenofovir alafenamide fumarate; Tenofovir diphospahte; Tenofovir disoproxil; Tenofovir disoproxil fumarate; Tenofovir exalidex; Tepotinib; Tepoxalin; Terameprocol; Terazosin; Terazosin hydrochloride; Terbinafine; Terbinafine hydrochloride; Terconazole; Terfenadine; Teriflunomide; Terodiline hydrochloride; Tesaglitazar; Tesmilifene; Testosterone; Testosterone propionate; Tetrabenazine; Tetracaine hydrochloride; Tetracycline hydrochloride; Tetradecylthioacetic acid; Tetramethylthiuram monosulfide; Tetrandrine; TG101209; TGX-221; Thenalidine; Theophylline; Thiabendazole; Thiamine diphosphate; Thiamine; Thiazolobenzimidazole; Thimerosal; Thioctic acid; thio-DABO 3s; thio-DABO 3t; thio-DABO 3w; Thioguanine; Thioridazine; Thioridazine hydrochloride; Thiosangivamycin; Thiram; Thonzonium bromide; Thymidine; Thymidine-5′-triphosphate; Thymol; Tiagabine hydrochloride; Tiaprofenic acid; Tibo; Tibolone; Tilorone dihydrochloride; Tioconazole; Tiopronin; Tiotixene; Tioxidazole; Tioxolone; Tipifarnib; Tiplasinin; Tipranavir; Tiratricol; Tivirapine; Tivozanib; Tizanidine; Tizoxanide; TMC 126; TMC647055; Tobramycin; Tolcapone; Tolnaftate; Tolonium chloride; Tolterodine; Toltrazuril; Tolvaptan; Tomivosertib; Topiramate; Topotecan; Topotecan hydrochloride; Toremifene; Toremifene citrate; Torsemide; Tozasertib; Trabectedin; Tranexamic acid; Tranilast; trans-Flupentixol; Trazodone; Trehalose; Tretazicar; Tretinoin; Triamterene; Tribromsalan; Trichlorfon; Trichostatin a; Triciribine; Triciribine phosphate; Triclabendazole; Triclocarban; Triclosan; Trifluoperazine; Trifluoperazine hydrochloride; Triflupromazine; Triflupromazine hydrochloride; Trifluridine; Triglycidyl isocyanurate; Trihexyphenidyl hydrochloride; Trimethoprim; Trimetrexate; Trimipramine; Trimipramine maleate; Triparanol; Tripelennamine citrate; Triprolidine; Triprolidine hydrochloride; Triptil; Troglitazone; Tromethamine; Tropisetron; Trovafloxacin mesylate; Trovirdine; Troxatyl; Trypaflavine hydrochloride; Tryptamine; Tryptophan; TSAO-m3T; Tsao-T; TTP-8307; Tubercidin; TYA5; Tyrosine; Tyrphostin B42; Ubenimex; UC-781; UK-201844; Ulixertinib; Ulodesine; UMI-77; Uprifosbuvir; Uracil; Urapidil hydrochloride; urea-PETT deriv. 13; Uric acid; Uridine; Uridine 5′-triphosphate; Uridine-5′-monophosphate; Ursolic acid; U.S. Pat. No. 9,107,954, maraviroc; Vadadustat; Vadimezan; Valacyclovir; Valdecoxib; Valganciclovir; Valine; Valopicitabine; Valproate; Valpromide; Valrubicin; Vandetanib; Vaniprevir; Vanoxerine; Vanoxerine dihydrochloride; Vapendavir; Vardenafil; Varenicline; Variamycin; Varlitinib; Vatalanib; Vatalanib dihydrochloride; VCH759; Vch759; vch 759; Vedroprevir; Velban; Veliflapon; Veliparib; Velpatasvir; Venetoclax; Venlafaxine; Verapamil; Verapamil hydrochloride; Verdinexor; Vergentan; Vesatolimod; Vicriviroc; Vidarabine; Vidofludimus; Vinblastine; Vincristine; Vinorelbine tartrate; Vinpocetine; Visudyne; vitamin B12; Vitamin e; Vitexin; Volasertib; Vorinostat; Voxilaprevir; VRX-480773; VX-702; Warfarin; Wedelolactone; Win vi; Win-54954; WIN61209; Xanthine; Xevinapant; XV638; Y-27632 dihydrochloride; Yangonin; Zafirlukast; Zalcitabine; Zaleplon; Zaltoprofen; Zanamivir; Zeaxanthin; Zidovudine; Zidovudine monophosphate; Zimeldine hydrochloride; Zimelidine dihydrochloride; Ziprasidone; Ziprasidone hydrochloride monohydrate; Ziresovir; Z-Leu-leu-leu-al; Z-L-Phe chloromethyl ketone; Zofenoprilat; Zolpidem; Zosuquidar; Zosuquidar trihydrochloride; Zotatifin; Zotepine; ZSTK474; vegetable or animal fats; ethyl alcohol; water; glycerin; propylene glycol; triethylene glycol; dimethylsulfoxide; solvents; betaine hydrochloride; caprilic acid; monolauric acid; monolaurin (glyceryl monolaurate); undecenoic acid (undecylenic acid); pau p'arco bark extract; cat's claw extract; garlic extract; black walnut shell extract; catalase enzyme; lipase; glucoamylase; pectinase; beta-glucanase; cellulase; alpha-galactosidase; amylase; invertase; xylanase; hemicellulase; or other enzymes; black cumin oil; nigella sativa; oregano oil; essential oils; lactoferrin; colloid silver; vitamin C; benfothiamine; sulbutiamine; allithiamine; vitamin D; vitamin A; iodine; melatonin; phenofibrate; glycine; N-acetylcysteine; lipoic acid; S-adenosyl-L-methionine; milk thistle extract; grape seed extract; carnitine; antiandrogens; bromhexine; cannabidiol; convalescent plasma; ensovibep; famotidine; iota-carrageenan; metformin; molnupiravir; bamlanivimab/etesevimab; bebtelovimab; casirivimab/imdevimab; sotrovimab; tixagevimab/cilgavimab; nitric oxide; paxlovid; peginterferon lambda; povidone-iodine; proxalutamide; camostat (camostat mesilate; camostat mesylate); foistar; Glycyrrhiza glabra; liquorice; Artemisia annua; hesperidin; umifenovir; kaempferol 3-o-rutinoside; catechin; epigallocatechin-3-gallate; nafamostat; nafamostat mesilate; nafamostat mesylate; omega 3; dha/epa; xylitol; xlear; apn01; alunacedase alfa; soluble ace2; rhace2-apn01; hydrogen peroxide; monolaurin; propolis; atovaquone; fenofibrate; fucoidan; hypochlorous acid; pomegranate; punica granatum; punicalagin; punicalan; urolithin a; rosuvastatin; selenium; theaflavin-3,3′-digallate; cryptoquindoline; losartan; myricitrin; chrysin; ro-0622; azd7442; tixagevimab; cilgavimab; cetirizine; ct-p59; darolutamide; doxycycline; masitinib; neem; azadirachta indica a. juss; regdanvimab; saline; selamectin; tannic acid; valproic acid; amentoflavone; pristimerin; cryptospirolepine; rhoifolin; pectolinarin; 6-gingerol; theaflavin; oleuropein; tacrolimus; puerarin; limonin; obacunone; sesamin; astragalin; aframomum melegueta; aprotinin; ashwagandha; withania somnifera; bedaquiline; bergamottin; boswellic acids; canakinumab; chlorpheniramine; chlorphenamine; chlorhexidine; clarinex; Echinacea purpurea; enzalutamide; forsythoside a; glycine; griffithsin; huashibaidu; jing si herbal tea; jinhua qinggan; jt001 (vv116); lenzilumab; lianhua qingwen; noq19; phosphodiesterase enzyme type 5 inhibitors; Pleurotus ostreatus; Rheum palmatum; s-217622; tafenoquine; tofacitinib; virgin coconut oil; xuanfeibaidu; moxidectin; thymoquinone; xanthoangelol e; tingenone; iguesterin; micafungin; emetine; colistin; glycyrrhizin; crocin; cryptomisrine; biscryptolepine; herbacetin; cycloeucalenol; campesterol; cyanidin-3-o-glucoside; afatinib; nilotinib; scedapin c; quinadoline b; afzelin; rhein; artesunate; bufalin; hydralazine; phenytoin; propylthiouracil; sulindac; bicuculline; tideglusib; bemcentinib; ergotamine; taraxerol; diosbulbinoside d; estrone-2,3-quinone; hinokiflavone; ginkgetin; norquinadoline a; natamycin; cyanidin 3-o-rutinoside; quercetin 3-o-rutinoside; phillyrin; conivaptan; aloesin; gingerol; tinocordiside; sesaminol; sesamolin; ephedrine; solanine; donepezil; vby-825; psoralidin; cryptotanshinone; fangchinoline; z-fa-fmk; allicin; pravastatin; ramipril; dihydromyricetin; scutellarein; corilagin; isorhamnetin; nystatin; anakinra; eculizumab; dieckol; sinigrin; glabridin; gimsilumab; kin1901; infliximab; leronlimab; pa14; pro-140; vyrologix; tocilizumab; procyanidin; α-hederin; ferulic acid; cyanidin; cyanidin 5-o-β-d-glucoside; cyanidin 3-o-glucoside; A-carrageenan; 4′-fluorouridine; 76clabs; 8g3; a6-001; agp-14; agp-15; acarbose; ard-61; arq ajib; arylazothiazolimines; aviptadil; ayurcov; beauvericin; bismuth subsalicylate; bis-thiadiazoles; blue light; brazilin; breathing exercises; brii-196/brii-198; bromelain; bucillamine; c60 fullerene; Camellia sinensis; cd24fc; chlorine dioxide; chyawanprash; clitoria ternatea; asian pigeonwings; bluebellvine; blue pea; butterfly pea; cordofan pea; darwin pea; codivir; copper(ii) gluconate; clsp-2; ctb-ace2 gum; cysteamine; d-α-tocopherol polyethylene glycol succinate; tpgs; dfo; diammonium glycyrrhizinate; dimethyl sulfoxide; darunavir ethanolate; ek1c4; engineered ace2; enoxaparin; epicatechin; fbr-002; estrogen; ethyl lauroyl arginine; exo-cd24; evusheld; tixagevimab; cilgavimab; ferrocene derivatives; flovid-20; gancao-banxia; gb-1; green tea; hanshi zufei; heparin; hinokitiol; homo-harringtonine; honey; inm005; interferon-A (interferon lamba); isoprinosine; js016; kabasura kudineer; kalmegh; led spirulina; levamisole; levilimab; 1-glutamine; ly2835219; manuka honey; maoto; mesencure; metformin glycinate; mi-1851; montelukast; multimeric soluble ace2; n-acetylglucosamine; natto extract; naphthoquine; niacinamide; nicotinamide mononucleotide; opaganib; oxygen-ozone immunoceutical therapy; palmitoylated ace2; pamapimod; pegylated interferon alpha-2b; peg ifn-α2b; pentosan polysulfate; pioglitazone; phoxwell; phthalocyanine; porphyridium sp.; pranayama; probenecid; propolis sulabiroin-a; pyramax; pyronaridine-artesunate; pyrimidine; q34; rd-x19; rejuveinix; sabizabulin; saliravira; sarbd-1; sars-block; schaftoside; sea cucumber sulfated polysaccharide; scsp; seraph 100 microbind affinity filter; serratiopeptidase; serratia e-15 protease; serralysin; serratiaprotease; serrapeptase; shallot; si-f019; sng001; spirulina; sodium bicarbonate; stenoparib; sti-9167; thymic peptides; tollovir; tpntl; trisb92; Ertica dioica agglutinin; zinv03977803; znonps; znsec-humicin; z-veid-fmk; minocycline; coclobine; dieckol 1; 3-isotheaflavin-3 gallate; dihydrotanshinone i; citriquinochroman; holyrine b; proximicin c; pityriacitrin b; anthrabenzoxocinone; penimethavone a; icatibant; bepotastine; epoprostenol; vapreotide; hrsace2; olysio; allyl disulphide; allyl trisulphide; digitoxigenin; tenufolin; pavetannin c1; lianhuaqingwen; deptropine; neostigmine bromide; ethotoin; hydrocotarnine; ampyrone; withanolide b; lurasidone; perfenazine; isocarboxazide; cryptophycin 1; cryptophycin 52; deoxycylindrospermopsin; anatibant; zabofoxacin; tiracizine; picotamide; cilazapril; indisulam; propadimine; phenformin; torososide b; covitris2020; chlovid2020; silybin; licoleafol; gardenin a; 6-paradol; Psiadia punctulata; tanshinone-i; mangiferin; γ-mangostin; 4-hydroxy-2-nonenal; acetaldehyde; aflatoxin-b1; aica-ribonucleotide; alitretinoin; alpha-tocopherol; benazepril; benzopyrene; bezafibrate; bisphenol-a; bucladesine; buthionine-sulfoximine; cadmium-chloride; carbon-tetrachloride; ciglitazone; cisplatin; clodronic-acid; clofibrate; colforsin; corticosterone; coumestrol; deguelin; dichloroacetic-acid; dieldrin; dimethylnitrosamine; dinoprost; ethinyl-estradiol; fumonisin-b1; furan; glafenine; glucosamine; ifosfamide; ionomycin; lithium-chloride; methapyrilene; methimazole; methoxychlor; mevalonic acid; ochratoxin-a; oxidopamine; pentachlorophenol; phenethyl-isothiocyanate; pifithrin; pilocarpine; pirinixic acid; piroxicam; pyrazolanthrone; ranitidine; rosiglitazone; tetrachloroethylene; tetracycline; thapsigargin; tributyltin; trichloroethylene; trichostatin-a; tunicamycin; tyrphostin-ag-1478; urethane; vancomycin; wortmannin; zearalenone; zinc32960814; zinc12006217; zinc03231196; nigricanoside a; nigricanoside b; callophysin a; gallocatechin; hyperin; lupinifolin; viomycin; capastat; demethoxycurcumin; bisdemethoxycurcumin; scutellarin; alloyohimbine gummadiol; asparagamine a; vincapusine; naringin; kanamycin; cefpiramide; salvianolic acid b; teniposide; benzoylgedunin; 6-deaminosinefungin; unii-o9h5kyllsv; cephalosporin derivatives; tcm5280805; tcm5280445; tcm5280343; tcm5280863; tcm5458190; quinadoline; polyketide isochaetochromin d1; 0-hydroxyusambarensine; 6-oxoisoiguesterin; 22-hydroxyhopan3-one; grazopavir; mfcd00832476; mfcd02180753; pacritinib; a3659; a3777; leucopelargonidin; eriodictyol; enterodiol; naringen; flemiflavanone d; euchrestaflavanone a; azadirachtin-h; azadirachtin-i; azadirachtin-q; sn00334175; sn00162745; alphaspinasterol; gycyrrhizin; azadirachtani; kushenol-w; cyanidin 5-o-β-d-glucoside; morelloflavone; methylochnaflavon; isoginkget; sciadopitysin; podocarpusflavone a; cryptomerin; cytochalasin z8; leucal; p-coumaric acid; ketazolam; methylnaltrexone; ethynodiol diacetate; petunidin 3,5-odiglucoside; delphinidin 3-o-rutinoside; las 51620435; las 51620429; rosoxacin; levomefolic; etodolac; tinofoviralafenamide; ornipressin; otosiban; lanreotide; argiprestocin; demoxytocin; carbetocin; lypressin; examorelin; polymyxin b1; verbascoside; abrisapogenol g; kaempferol-3-o-rutinoside; hederagenin; abrusoside a; robustaflavone; agathisflavone; swertianolin; ononin; pedunculoside; imperialine; peimisine; batatasin i; pomiferin; coumarin; rhodionin; aloin; sarsasapogenin; silydianin; morusin; α-mangostin; yohimibine; coclaurine; strychnine; myristicin; medicarpin; coptisine; tiliroside; glabrone; lignans; gastrodin; ajugol; evodiamine; nuciferine; cynarin; gramine; narciclasine; protopanaxatriol; karanjin; gigantol; harsingar; nictoflorin; lupeol; aloevera; aloenin; giloy; sitosterol; nimbin; ginger; shogaol; cyanin; medicagol; faradiol; flavanthrin; withanoside v; somniferine; vicenin; isorientin; sesamolinol; ecteinascidin; homoharringtonine; dolastatin; halichondrin; plicamycin; arvoside; nicotiflorin; kaempferol sulphates; quercetin-3-o-glucuronide; isorhamnetin-3-o-glucoside; narcissin; calendula glycoside b; lauruside; miricetine-3-glucoside; cordifoliside; anthraquinones; emodine 8-glucosides; acetoside; lisinopril; deoxynortryptoquivaline; thalimonine; sophaline; tomatidine; 10-hydroxyusambarensine; stychnopentamine; usambarensine; vanillin; crambescidin 786; crambescidin 826; sepiapterin; tetrodotoxin; caulerpin; lutein; galanthamine; nomilin; deacetylnomilin; ichangin; amyrin; 24-dimethylene cycloartenol; isoiguesterin; Lentinula edodes; Agaricus bisporus; jmx0286; jmx0301; jmx0941; chloramphenicol; mpi43; mpi44; mpi46; keigairengyoto; shosaikotokakikyosekko; kakkontokasenkyushin'i; vuwcov059; vwcov267; vuwcov270; 4-hydroxycordoin; mallotophilippen d; 3′-(3-methyl-2-butenyl)-4′-o-β-d-glucopyranosyl-4,2′-dihydroxychalcone; lefamulin; cefpodoxime; ciprofloxacin; sparfloxacin; moxifloxacin; tbaj-876; (s)-crizotinib; spermidine; spermine; saffron; mdl-28170; z Ivg chn2; ono 5334; mln-3897; bavachin; isobavachalcone; lycorine hydrochloride; lycorine; bufotaline; cinobufagin; periplocoside; veratridine; coniferyl aldehyde; cornuside; brusatol; momordinic; roburicacid; hruceine a; isoalantolactone; oridonin; dehydrocostus lactone; alantolactone; dehydrodiisoeugenol; liensinine; isoliensinine; vps34-in1; stf-62247; mcoppb; gw 803430; amodiaquine dihydrochloride; n-methylspiperone; gmc 2-29; lu ae58054; methdilazine; methotrimeprazine maleate; difeterol; naltrindole isothiocyanate; am1241; cpdd; sb 271046; gmc 2-113; caa-0225; cathepsin inhibitor 1; z-gly-leu-phechloromethyl ketone; balicatib; calpain inhibitor i; ikk-2 inhibitor viii; nsc 33994; m1414; it1t dihydrochloride; s-15176 difumarate; jtv519 hemifumarate; rescimetol; trifluomeprazine 2-butenedioate; asteriscunolide d; genz-123346; melitracen; dlunarizine; proglumetacin; dmp 777; dexanabinol; trifluoperazine 2hcl; thioridazine hcl; bicalutamide; cinnamaldehyde; piperine; zingeberene; gr 127935; levetiracetam; sotalol; oxazepam; thiamazole; hydroxycarbamide; clonazepam; meloxicam; temazepam; anastrozole; perindopril; silodosin; lorazepam; bupropion; bisoprolol; linagliptin; clopidogrel; allopurinol; indapamide; chlortalidone; phylloflavan; milk thistle; ilexin b; isosilybin b; spiperone; vitamin b9; vitamin k1; vitamin k2; oleanderolide; proceragenin a; balsaminone a; anacardic acid; aloeresin d; tcid; dihydrokaempferol; dihydroquercetin; myricentin; isoquercitrin; pc000550; pc000361; pc000558; pc000573; gr10617; quercetagetin; glycycrrhizin; 59-tetrahydrocannabinol; arteether; dihydroartemisinin; arteannuin; cannabidinol; punicalin; panduratin a; Andrographis paniculata; Zingiber officinale; Boesenbergia rotunda; Scutellaria baicalensis; pomegranate peel extract; zinc000013444414; zinc000137976768; zinc000143375720; diosmin; apiin; okadaic acid; p-57as3; concanamycin a; oleandrin; gitoformate; eprinomectin; fusicoccin; perilla aldehyde; perillyl alcohol; valaciclovir; finasteride; betamethasone; clonidine; somatotropin; nitisinone; tadalafil; etacrynic acid; proguanil; tamsulosin; rifapentine; cyclosporine; nitrogen; arginine; abatacept; interferon beta-la; thalidomide; crizanlizumab; nitroglycerin; brexanolone; omalizumab; siltuximab; propranolol; interferon beta-1b; prednisolone; torin-2; rapamycin; radotinib; thiostrepton; cl-amidine; bb-cl-amidine; broussoflavan a; hygromycin b; nabiximols; abyssinone ii; procynidin b1; indigo blue; crytospirolepine; 10′-hydroxyusambaresine; strychnopentamine; usararotenoid a; 12α-epi-milletosin; torchnil; febcin; 5-chloro-omega-hydroxy-1-o-methylemodin; cystodion e; jq1; zbc260; canderastan cilexetil; saquanivir; boron citrate; oleoylethanolamide; liquiritin; laminarin; eckol; trifucol; β-d-galactose; K-carrageenan; c135-Is/c144-ls; sab-185; vir-7831/vir-7832; covi-amg/covi-drops; covi-guard; 2-deoxy-d-glucose; dnl758; lanadelumab; abivertinib; bld-2660; pemziviptadil; artesunate/pyronaridine; carragelose; at-527; ptc299; brilacidin; cigb-325; sb203580; mapk13-in-1; arry-797; tofacitinibfedratinib; mpro 13b; gc-373; gc-376; mpro n3; fedratinib; ipratropium bromide; lomitapide; metoclopramide; s1ra; crinine; ilexsaponin b2; strictinin; zinc000027215482; zinc000252515584; loniflavone; 2′-o-ribose methyltransferase; tirucallin a; zinc000253504770; zinc000253504766; triamcinolone; hydrochlorothiazide; terflavin a; chebulagic acid; chebulinic acid; coumaroylquinic acids; sinapoyl d glucoside; tetra-o-galloyl-β-d-glucose; methyl rosmarinates; cosmosiin; dihydronitidine; lectin; digitoxigenine; calarene; amaranthin; diarylheptanoids; indigo; aloe emodin; dihydrocelastrol; phyllaemblinol; isocolumbin; magnoflorine; piperolactam a; withanone; biflavone; cinnamic amides; agomelatine; ramelteon; immunoglobulin; ifn-β1a; ifn-β1b; interleukin-2; cynk-001; asc09; cobicistat; carrimycin; dihydroartemisinine; piperaquine; piclidenoson; cf101; nivolumab; obtivo; meplazumab; jakotinib; tj003234; tozumab; adamumab; ravulizumab; alxn1210; clazakizumab; avdoralimab; iph5401; ly3127804; ifx-1; bevacizumab; valsartan; 7-hydroxystaurosporine; bafetinib; β-glucan; p-coumaroyltriacetic acid lactone; zinc02111387; zinc02122196; sn00074072; zinc04090608; xuebijing; lung cleansing and detoxifying decoction; etanercept; enalapril; oroxylin a; irisolidone; anhydrosafflor yellow b; euchrenone; uncaric acid; demethylzeylasteral; maslinic acid; atractylenolide iii; astragaloside iv; daturaolone; cucurbitacin g 2-glucoside; citronellol; limonene; salvianolic acid; neochlorogenic acid; kobophenol a; bis-demethoxycurcumin; 5-viniferin; pseudoephedrine; methylephedrine; speciophylline; uncarine f; anisodamine; forsythoside i; amygdalin; urso-deoxycholic acid; withanolide a; β-sitosterol; farnesiferol b; dexchlorpheniramine; tanshinones; isatis indigotica root; rhizoma cibotii; torreya nucifera; shuang huang lian; eugenol; ubiquinone; poly-methoxyflavones; poly-hydroxyflavones; mearnsitrin; 3-o-β-d-glucoside; biorobin; delphinidin; galangin; isoferulic acid; dithymoquinone; negillicine; negillidine; muc1; α-lactalbumin; monoclonal antibody; or derivatives thereof; or mixtures thereof.

### V.II. Section

In alternative embodiments additional therapeutic agent can be formulated in any way and can be applied in a variety of forms including nanoparticles, polymeric nanoparticles (PNPs), liposomes, freeze-dried liposomes, micelles, polymeric micelles, niosomes, solid lipid nanoparticles (SLNs), nanostructured lipid carriers (NLCs), nanoemulsions, emulsions, self-nanoemulsifying drug delivery systems (SNEDDS), nanocrystals, co-crystals, mesoporous silica nanoparticles (MSNs), dendrimers, ultradispersions, solid dispersions, noncovalent complexes, complexes with polymer, complexes with biopolymer, solutions, solutions in fat/oil, solutions with cosolvent, solutions with surface active agent/surfactant, solutions with solubilizing agent, polymorphes, lipid-based systems, gels, colloids, sols, or in any form described in the scientific and patent literature and obtained in any of the ways described in the scientific and patent literature.

In alternative embodiments synergistic effects for FORMULA 1-111 can be achieved with niclosamide, remdesivir, etc. in a variety of forms. Other synergist can be found in the scientific and patent literature.

In alternative embodiments additional agents such as verapamil, polyethoxylated castor oil derivatives, the emulsifying agent Cremophore RH 60 and tween 80, grapefruit juice, etc. (other agents can be found in the scientific and patent literature), which are modulators for CYP3A4 and P-glycoprotein, can increase the absorption of FORMULA 1-111 compounds.

In another aspect, provided is a method for the treatment (and for use in manufacturing a pharmaceutical dosage form to treat) of viral diseases in a human or mammal or animal or bird in need thereof comprising administering a therapeutically effective amount of a stabilized aqueous formulation which comprises avermectin, surface active agent and cosolvent.

In another aspect, provided is a method for the treatment (and for use in manufacturing a pharmaceutical dosage form to treat) of viral diseases in a human or mammal or animal or bird in need thereof comprising administering a therapeutically effective amount of a stabilized formulation which comprises avermectin solutions in fat/oil or avermectin emulsion.

In another embodiment, the method for the treatment (and for use in manufacturing a pharmaceutical dosage form to treat) of diseases in a human or mammal or animal or bird in need thereof comprises administering a therapeutically effective amount of a pharmaceutical composition comprising an effective amount of a FORMULA 1-11 compound in combination with at least one additional therapeutic agent.

In another embodiment, a method for the treatment (and for use in manufacturing a pharmaceutical dosage form to treat) of diseases in a human or mammal or animal or bird in need thereof includes administering a therapeutically effective amount of a pharmaceutical composition comprising an effective amount of compound of FORMULA 1-111 in combination with at least one additional therapeutic agent such as (but not limited to) intravenous or intramuscular forms of thiamine, vitamin C, vitamin D, glutanione, S-adenosylmethionine, cysteine, N-acetylcysteine.

### V. FORMULATIONS AND PHARMACEUTICAL COMPOSITIONS

The compounds of the present invention are formulated with common carriers and excipients, which are selected according to common practice. Tablets will contain excipients, glydants, fillers, binders, and the like. Aqueous formulations are prepared in sterile form, and if they are not intended to be administered orally, they are usually isotonic. All formulations optionally contain excipients, such as those described in Handbook of Pharmaceutical Excipients (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is usually between 7 and 10. In some embodiments of the invention, the pH of the formulations varies from about 2 to about 5, but is usually about 3 to 4.

Although the active ingredients may be administered separately, it may be preferable to present them as pharmaceutical compositions. Compositions of the invention, whether for veterinary or human use, include at least one active ingredient, as defined above, together with one or more acceptable carrier(s) for it and, optionally, other therapeutic ingredients, in particular additional therapeutic ingredients, which are discussed herein. The carrier(s) must be “acceptable” in the sense of being compatible with the other ingredients of the composition and physiologically harmless to the recipient.

The compositions include those suitable for the above-mentioned routes of administration. The compositions may be conveniently presented in unit dose form and may be prepared by any of the methods well known in the pharmaceutical art. The methods and compositions are generally described in Remington's Pharmaceutical Sciences (Mack Publishing Co., Easton, Pa.). Such methods involve the step of bringing into association the active ingredient with the carrier which constitutes one or more accessory ingredients. In general the formulations are prepared by uniformly and intimately bringing into association the active ingredient with liquid carriers or finely divided solid carriers or both, and then shaping the product if necessary.

Formulations of the present invention suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can be administered as a bolus, electuary or paste.

Tablets are made by compression or molding, optionally with one or more additional ingredients. Compressed tablets can be prepared by compressing in a suitable machine an active ingredient in a free-flowing form, such as a powder or granules, further mixed with a binder, lubricant, inert diluent, preservative, surface-active or dispersing agent. Moulded tablets can be made by molding in a suitable machine a mixture of a powdered active ingredient moistened with an inert liquid diluent. The tablets may additionally be coated with a shell or pellets and optionally formulated to allow slow or controlled release of the active ingredient therefrom.

For infections of the eyes or other external tissues, such as the mouth and skin, the compositions are preferably applied in the form of an ointment or cream containing the active ingredient(s) in an amount, for example, from 0.075 to 20% wt. (including the active ingredient(s) in a range of 0.1% to 20% in 0.1% wt. increments, e.g., 0.6% wt., 0.7% wt., etc.), more preferably from 0.2 to 15% wt. and most preferably from 0.5 to 10% wt. When formulated as an ointment, the active ingredients may be used with a paraffin or water-miscible ointment base. As an alternative, the active ingredients may be formulated as a cream with an oil-in-water type cream base.

Optionally, the aqueous phase of the cream base may include, if desired, at least 30% wt. of a polyhydric alcohol, that is, an alcohol with two or more hydroxyl groups, such as propylene glycol, butane-1,3-diol, mannitol, sorbitol, glycerol, polyethylene glycol (including PEG 400) and mixtures thereof. Compositions for topical use may preferably include a compound that enhances absorption or penetration of the active ingredient through the skin or other affected areas. Among examples of such dermal penetration enhancers are dimethyl sulfoxide and related analogues.

The oil phase of emulsions of the present invention can be composed of known ingredients in a known manner. Although the phase may comprise an emulsifier alone (otherwise referred to as emulsifier), preferably it comprises a mixture of at least one emulsifier with a fat or oil or with a fat and oil. The hydrophilic emulsifier is preferably included together with a lipophilic emulsifier, acting as a stabilizer. Also preferably both oil and fat are included. Emulsifier(s) with or without stabilizer(s) together constitute the so-called emulsifying wax, and the wax together with oil and fat constitute the so-called emulsifying ointment base, which forms the oily dispersed phase of the cream compositions.

Emulsifiers and emulsion stabilizers appropriate for use in the formulation of the invention include Tween® 60, Span® 80, cetostearyl alcohol, benzyl alcohol, myristyl alcohol, glyceryl monostearate and sodium lauryl sulfate. Other emulsifiers and emulsion stabilizers appropriate for use in the formulation of the invention include Tween® 80.

The choice of suitable oils or fats for the formulation is determined by achieving the desired cosmetic properties. The cream may be a non-greasy, non-staining and washable product with a suitable consistency to avoid leakage from tubes or other containers. Straight or branched chain alkyl esters, mono- or dibasic, such as di-isoadipate, isocetyl stearate, propylene glycol coconut fatty acid diester, isopropyl myristate, decyl oleate, isopropyl palmitate, butyl stearate, 2-ethylhexyl palmitate or a mixture of branched chain esters known as Crodamol CAP, the latter three being preferred. They may be used alone or in combination depending on the desired properties. As an alternative, high melting point lipids such as white soft paraffin and/or liquid paraffin or other mineral oils are used.

According to the present invention, pharmaceutical compositions include a combination together with one or more pharmaceutically acceptable carriers or excipients and optionally other therapeutic agents. The pharmaceutical compositions containing the active ingredient may be in any form suitable for the intended route of administration. For oral administration, the compositions may be prepared, for example, tablets, trochets, lozenges, aqueous or oily suspensions, dispersible powders or granules, emulsions, hard or soft capsules, syrups or elixirs. Formulations intended for oral use may be made according to any method known in the art for making pharmaceutical compositions, and such compositions may contain one or more agents, including sweeteners, flavorings, colorings, and preservative agents, to provide a palatable preparation. Tablets containing the active ingredient in a mixture with a non-toxic pharmaceutically acceptable excipient that is suitable for tablet production are acceptable. Such excipients may be, for example, inert diluents such as calcium or sodium carbonate, lactose, calcium or sodium phosphate; granulating and disintegrating agents such as corn starch or alginic acid; binding agents such as starch, gelatin or acacia; and lubricating agents such as magnesium stearate, stearic acid or talc. Tablets may be uncoated or may be coated using known methods, including microencapsulation, to delay breakdown and adsorption in the gastrointestinal tract and thereby provide sustained action over an extended period. A time delayed material such as glyceryl monostearate or glyceryl dystearate alone or with a wax, for example, may be used.

Products for oral use can also be designed as hard gelatin capsules where the active ingredient is mixed with an inert solid diluent, such as calcium phosphate or kaolin, or as soft gelatin capsules where the active ingredient is mixed with water or an oil medium, such as peanut oil, liquid paraffin or olive oil.

Aqueous suspensions according to the invention contain active materials mixed with excipients suitable for making aqueous suspensions. Such excipients may include a suspending agent such as sodium carboxymethylcellulose, methylcellulose, hydroxypropyl methylcellulose, sodium alginate, polyvinylpyrrolidone, tragacanth gum and acacia gum, and dispersing or wetting agents such as naturally occurring phosphatide (such as lecithin), an alkylene oxide condensation product with a fatty acid (e.g., polyoxyethylene stearate), an ethylene oxide condensation product with a long-chain aliphatic alcohol (e.g., hepta dekaethylene acetanol), an ethylene oxide condensation product with a partial ester derived from a fatty acid and hexitol anhydride (e.g., polyoxyethylene sorbitan monoleate). The aqueous suspension can also contain one or more preservatives such as ethyl- or n-propyl p-hydroxy-benzoate, one or more colorants, one or more flavorings, and one or more sweeteners such as sucrose or saccharin. Other non-limiting examples of suspending agents include Cyclodextrin and Captisol.

Oil suspensions can be prepared by suspending the active ingredient in a vegetable oil such as peanut oil, olive oil, sesame oil or coconut oil, or in a mineral oil such as liquid paraffin. A thickening agent such as beeswax, solid paraffin or cetyl alcohol may be contained in the oral suspension. Sweeteners, such as those described above, and flavorings may be added to produce a palatable oral preparation. These compositions may be preserved by adding an antioxidant such as ascorbic acid.

Dispersible powders and granules of the invention, applicable for preparing or manufacturing an aqueous suspension by adding water, include the active ingredient in a mixture with a dispersing or wetting agent, a suspending agent and one or more preservatives. Suitable dispersing or wetting agents and suspending agents are examples of those disclosed above. Additional excipients, such as sweetening, loosening and coloring agents, may also be present.

The pharmaceutical compositions of the invention can also be presented as oil-in-water emulsions. The oil phase can be a vegetable oil such as olive or peanut oil, a mineral oil such as liquid paraffin, or a mixture thereof. Appropriate emulsifiers include gum of natural origin such as acacia gum and tragacanth gum, phosphatides of natural origin such as soy lecithin, esters or partial esters derived from fatty acids and hexitol anhydrides such as sorbitan monooleate, and condensation products of these partial esters with ethylene oxide such as polyoxyethylene sorbitan monooleate. The emulsion can also contain sweeteners and flavorings. Syrups and elixirs may contain sweetening agents such as glycerin, sorbitol, or sucrose. These formulations can also contain a demulgent, preservative, flavoring, or coloring agent.

The pharmaceutical compositions of the invention may be in the form of a sterile injectable preparation, such as a sterile injectable aqueous or oily suspension. This suspension may be prepared according to the prior art using suitable dispersing or wetting agents and suspending agents as mentioned above. The sterile injectable preparation may also be a sterile injectable solution or suspension in a non-toxic parenterally acceptable diluent or solvent, such as a solution in 1,3-butanediol or prepared as a lyophilized powder. Acceptable vehicles and solvents that may be used include water, Ringer's solution, and isotonic sodium chloride solution. In addition, sterile fixed oils are usually used as a solvent or suspension medium. Any tasteless fixed oil can be used for this purpose, including synthetic mono- or diglycerides. In addition, fatty acids, such as oleic acid, can also be used to prepare injectable preparations. Among the acceptable vehicles and solvents that can be used are water, isotonic Ringer's solution, isotonic sodium chloride solution and hypertonic sodium chloride solution.

The amount of active ingredient that can be combined with a carrier material to produce a single dosage form will vary depending on the recipient and the particular route of administration. For example, a time-release formulation intended for oral administration to humans may contain from about 1 to 1000 mg of the active ingredient combined with an appropriate and convenient amount of carrier material, which may be from about 5 to about 95% of the total amount of the composition (weight:weight). The pharmaceutical composition may be prepared so as to provide an easily measurable amount for administration. For example, an aqueous solution intended for intravenous infusion may contain from about 3 to about 500 μg of active ingredient per milliliter of solution to provide an infusion of a suitable volume at a rate of about 30 ml/hr.

Formulations suitable for topical administration to the eye also include eye drops in which the active ingredient is dissolved or suspended in a suitable carrier, especially in an aqueous solvent for the active ingredient. The active ingredient is preferably present in such compositions in a concentration of from 0.5 to 20%, more preferably from 0.5 to 10% and especially about 1.5% w/w.

Formulations suitable for topical use in the mouth include lozenges containing the active ingredient in a flavored base, usually sucrose and acacia or tragacanth; lozenges containing the active ingredient in an inert base such as gelatin and glycerin or sucrose and acacia; and mouthwashes containing the active ingredient in a suitable liquid carrier.

Formulations for rectal administration may be in the form of a suppository with a suitable base including, for example, cocoa butter or salicylate.

Formulations suitable for intrapulmonary or nasal administration have particle sizes, for example, in the range of 0.1 to 500 microns, such as 0.5, 1, 30, 35, etc., which are administered by rapid inhalation through the nasal passage or inhalation through the mouth to reach the alveolar sacs. Suitable compositions include aqueous or oily solutions of the active ingredient. Formulations suitable for administration as an aerosol or dry powder may be prepared according to conventional methods and may be delivered together with other therapeutic agents, such as compounds previously used to treat or prevent viral infections or viral diseases, as described below.

Formulations suitable for vaginal administration may be in the form of pessaries, tampons, creams, gels, pastes, foams or sprays containing in addition to the active ingredient such carriers as are known in the art to be suitable.

Formulations suitable for parenteral administration include aqueous and non-aqueous sterile solutions for injection, which may contain antioxidants, buffers, bacteriostatics and solvents that make the formulation isotonic with the blood of the intended recipient; and aqueous and non-aqueous sterile suspensions, which may include suspending and thickening agents.

The formulations are presented in single or multi-dose containers, such as sealed ampoules and vials, and can be stored in a freeze-dried (lyophilized) state requiring only the addition of a sterile liquid carrier, such as water for injection, immediately prior to use. Extemporaneous injectable solutions and suspensions are made from sterile powders, pellets, and tablets described earlier. Preferred single-dose preparations are preparations containing a daily dose or a single daily subdose, as described above, or an appropriate fraction of the active ingredient.

It is important to note that in addition to the ingredients especially mentioned above, the compositions of the present invention may include other agents common in the art, taking into account the type of composition in question, for example, compositions suitable for oral use can include flavorings.

The invention also provides veterinary compositions comprising at least one active ingredient, as defined above, together with a veterinary carrier for it.

Veterinary carriers are materials useful for administering the composition and may be solids, liquids, or gases that are inert or acceptable in veterinary medicine and compatible with the active ingredient. These veterinary compositions may be administered orally, parenterally, or by any other desired route.

Compounds of the invention are used to make controlled-release pharmaceutical compositions containing as an active ingredient one or more compounds of the invention (“controlled-release compositions”) in which the release of the active ingredient is controlled and regulated to provide less frequent dosing or to improve the pharmacokinetic or toxicity profile of that active ingredient.

In another embodiment, the method of treating (and for use in manufacturing a pharmaceutical dosage form to treat) a disease in a human in need thereof comprises administering a therapeutically effective amount of a pharmaceutical composition comprising an effective amount of a FORMULA I-Ill compound in combination with a pharmaceutically acceptable diluent or carrier.

In another embodiment, the method comprises administering a therapeutically effective amount of a combination pharmaceutical agent (of FORMULA IV) in form of solution/emulsion/suspension of comprising:
a) a first pharmaceutical composition comprising a compound of FORMULA 1-111, and
b) at least one of the following components:
(1) oil phase containing vegetable or/and animal fats;
(2) one or more surfactants;
(3) one or more solvents;
(4) one or more gelling agents.

FORMULA IV can be a true solution or colloidal solution, emulsion, solid or liquid dispersion, suspension, emulsion-suspension form, gas cocktail, foam, sol, gel, paste, their mixture. A FORMULA IV can be prepared by mixing, homogenizing, dispersing, and emulsifying the constituent components.

In another embodiment, the method comprises administering a therapeutically effective amount of a combination pharmaceutical agent (of FORMULA IV variant A) comprising:
(a) oil phase containing vegetable or/and animal fats or/and lecithin 1-99.999% by weight
(b) a compound of FORMULA I-II or/and “API” 0.001-99% by weight
(c) (optional) one or more surfactants 0-70% by weight (physiologically acceptable surfactant selected from the surfactants listed in LINK [0061]F (III.I. Section)).
(d) (optional) one or more solvents 0-99.9% by weight (physiologically acceptable solvent selected from the solvents listed in LINK [0061]Method 1 (III.I. Section)).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

In another embodiment, the method comprises administering a therapeutically effective amount of a combination pharmaceutical agent (of FORMULA IV variant B) comprising:
(a) (optional) oil phase containing vegetable or/and animal fats or/and lecithin 0-99.999% by weight
(b) a compound of FORMULA I-II or/and “API” 0.001-99% by weight
(c) (optional) one or more surfactants 0-70% by weight (physiologically acceptable surfactant selected from the surfactants listed in LINK [0061]F (III.I. Section)).
(d) one or more solvents 5-99.9% by weight (physiologically acceptable solvent selected from the solvents listed in LINK [0061]Method 1 (III.I. Section)).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

In another embodiment, the method comprises administering a therapeutically effective amount of a combination pharmaceutical agent (of FORMULA IV variant C) comprising:
(a) oil phase containing vegetable or/and animal fats or/and lecithin 1-99.999% by weight
(b) a compound of FORMULA I-II or/and “API” 0.001-99% by weight
(c) (optional) one or more surfactants 0-70% by weight (physiologically acceptable surfactant selected from the surfactants listed in LINK [0061]F (III.I. Section)).
(d) one or more solvents 1-99.9% by weight (physiologically acceptable solvent selected from the solvents listed in LINK [0061]Method 1 (III.I. Section)).
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

In another embodiment, the method comprises administering a therapeutically effective amount of a combination pharmaceutical agent (of FORMULA IV variant D) comprising:
(a) (optional) oil phase containing vegetable or/and animal fats or/and lecithin 0-99.999% by weight;
(b) a compound of FORMULA I-II or/and “API” 0.001-99% by weight;
(c) one or more surfactants 0.05-70% by weight (physiologically acceptable surfactant selected from the surfactants listed in LINK [0061]F (III.I. Section));
(d) (optional) one or more solvents 0-99.9% by weight (physiologically acceptable solvent selected from the solvents listed in paragraph LINK [0061]Method 1 (III.I. Section));
(e) (optional) one or more gelling agents (0-99.9% by weight) include (but are not limited to): alginate, pectin, carrageenan, gellan, gelatin, agar, modified starch, methyl cellulose, and hydroxypropylmethylcellulose.

The active compounds of FORMULA I-IV can be administered/can be in form of foods, food additives, edible soluble films, drinks, medicinal agents, and feeds for domestic and wild animals. The drinks may be non-alcohol drinks or alcohol drinks. Examples of non-alcohol drinks include carbonated drinks, noncarbonated drinks (such as fruit juice, and nectar), soft drinks, sports drinks, tea, coffee, and hot chocolate. The alcohol drinks may be in the form of, for example, beer, low-malt beer, third-category beer, sake, wine, champagne, liqueur, or medicated liquor. For use as a food material or food additive (e.g., human food or animal food, such as a dog or cat food or feed for poultry, cows, or pigs), the active compound may be in the form of, for example, a tablet, a capsule formulation, a solid agent (such as a powder and a granule) dissolved in drinks, a semi-solid such as jelly, a liquid (such as drinking water), and a high-concentration solution diluted before use. Optional components, such as vitamins, carbohydrates, dyes, and flavoring agents commonly added to food may be appropriately mixed. The food may be given in any form, including a liquid and a solid.

In another embodiment, the method comprises administering a therapeutically effective amount of a combination pharmaceutical agent (of FORMULA V) in form of food product or beverage, or dietary supplement.

In alternative embodiments, the invention provides pharmaceutical formulations or compositions for use in in vivo, in vitro or ex vivo methods to treat, prevent, reverseand or ameliorate a medical conditions, for example, disease, disorder, syndrome, infection.

In alternative embodiments, the pharmaceutical compositions as provided herein or used to practice methods as provided herein can be administered parenterally, topically, orally or by local administration, such as by aerosol or transdermally. These pharmaceutical compositions can be formulated in any way and can be administered in a variety of unit dosage forms depending upon the condition or disease and the degree of illness, the general medical condition of each patient, the resulting preferred method of administration and the like. Details on techniques for for mulation and administration are well described in the scientific and patent literature, see, for example, the latest edition of Remington's Pharmaceutical Sciences, Maack Publishing Co., Easton Pa. (“Remington's”). For example, in alternative embodiments, these compositions of the invention are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods as provided herein can be in forms such as tablets, pills, powders, capsules, liquids, gels, syrups, slurries, suspensions, etc., suitable for in vivo, in vitro or ex vivo applications.

In alternative embodiments, the compositions can be formulated in any way and can be applied in a variety of forms including solid dispersions, polymeric nanoparticles (PNPs), liposomes, micelles, niosomes, solid lipid nanoparticles (SLNs), nanostructured lipid carriers (NLCs), nanoemulsions, emulsions, self-nanoemulsifying drug delivery systems (SNEDDS), nanocrystals, mesoporous silica nanoparticles (MSNs) and dendrimers; and/or using methods of:

| | |
|---|---|
| Physical | A) Reduction Particle size |
| modification | a) Micronization |
| | b) Nanosuspension, |
| | B) Modification of the crystal habit |
| | C) Solid dispersions |
| | a) Eutectic mixtures |
| | b) Solid solutions |
| | c) Amorphous solid solutions |
| | d) Glass solutions and glass suspension |
| | e) Cryogenic techniques. |
| Chemical | A) Change of pH, |
| modification | B) Use of buffer, |
| | C) Derivatization, |
| | D) Complexation, |
| | E) Salt formation. |
| Miscellaneous | A) Supercritical fluid process, |
| methods | B) Use of adjuvant like surfactant, solubilizers, |
| | cosolvency, hydrotropy, and novel excipients. |

or in any way described in the scientific and patent literature.

### VI. ROUTES OF ADMINISTRATION

The compounds of FORMULA I-V described herein may be administered (e.g., orally) in the form of a solid or liquid dosage form. In both, the compounds may be coated in a material to protect them from the action of acids and other natural conditions which may inactivate the compounds. The compounds may be formulated as aqueous solutions, liquid dispersions, (ingestible) tablets, buccal tablets, troches, capsules, elixirs, powders, granules, ointments, adhesive skin patches, sprays, suspensions, syrups, and wafers. The dosage forms may include pharmaceutically acceptable excipients, diluents, and/or carriers known in the art, such as binders, disintegrating agents, emulsifiers, lubricants, flavorants, antioxidants, and preservatives. Liquid dosage forms may include diluents such as saline or an aqueous buffer.

Any route of administration FORMULA I-V may be selected for use in the methods described herein. For instance, the route of administration may be selected from oral, nasal, topical, buccal, rectal, vaginal, ophthalmic, subcutaneous, intramuscular, intraperitoneal, epidural, intravenous, intraarterial, intratumoral, spinal, intrathecal, intra-articular, intra-arterial, sub-arachnoid, sublingual, oral mucosal, pulmonary, bronchial, lymphatic, intra-uterine, subcutaneous, intratumor, integrated on an implantable device, intradural, intracortical, intradermal, dermal, epidermal, transdermal, vaginal, rectal, ocular (for examples through the conjunctiva), intraocular, uretal, and parenteral. An advantage of the compounds of this invention is that they are orally bioavailable and can be dosed orally. A preferred route of administration is oral.

In the methods of the present invention for treating (and for use in manufacturing a pharmaceutical dosage form to treat) viral infection, compounds of the present invention may be administered at any time to a person who may be in contact with people suffering from viral infection or already suffering from viral infection. In some embodiments, the compounds of the present invention can be administered prophylactically to people in contact with people suffering from viral infection. In some embodiments of the invention, the compounds of the present invention can be administered to people who test positive for viral infection but do not yet have symptoms of viral infection. In some embodiments of the invention, the compounds of the present invention can be administered to people after symptoms of viral infection appear.

The effective dose of the active ingredient depends at least on the nature of the disease being treated, the toxicity, whether the compound is used prophylactically (lower doses) or against an active viral infection, the method of delivery and the pharmaceutical formulation, and is determined by the physician using routine dose escalation studies. It can be expected to range from about 0.0001 to about 100 mg/kg body weight per day; typically from about 0.01 to about 10 mg/kg body weight per day; more typically from about 0.01 to about 5 mg/kg body weight per day; most typically from about 0.2 to about 1 mg/kg body weight per day. For example, a daily candidate dose for an adult with a body weight of about 70 kg would be from about 1 mg to 1000 mg, preferably from 5 mg to 500 mg, and may take the form of a single or multiple dose. All dosages are given for an equivalent amount of pure “API”.

The effective dose of the compound of the present invention for treating Viral infection may depend on whether it is used prophylactically or to treat a person already suffering from viral infection. Moreover, the dose may depend on whether the person suffering from viral infection is not yet showing symptoms or is already showing symptoms of viral infection. People who test positive for viral infection and people with symptoms of viral infection may require higher doses for treatment compared to people receiving preventive treatment.

Any suitable time period for administration of the compounds of the present invention is contemplated. For example, the administration may be from 1 day to 100 days, including 2, 3, 4, 5, 6, 7, 8, 9, 10, 15, 20, 25, 30, 35, 40, 50, 60, 70, 80 or 90 days. Admission may also last from 1 week to 15 weeks, including 2, 3, 4, 5, 6, 7, 8, 9, 10, 11, 12, 13 or 14 weeks. Longer periods of administration are also suggested. The timing of administration may depend on whether the compound is administered prophylactically or to treat a person suffering from viral infection. For example, prophylactic administration may be for a period of time while the person is in constant contact with other people suffering from viral infection and for a suitable period of time after the last contact with the person suffering from Viral infection. For people already suffering from Viral infection, the period of use may be any length of time necessary to treat the patient and a suitable period of time after a negative test for viral infection to ensure that viral infection will not return.

### VII. EXAMPLES

### A. Preparation of Compounds

### Example 1

Ivermectin of pharmaceutical grade, CAS Number 70288-86-7, was purchased from Aecochem Corp., Xiamen, China. Apple pectin was purchased from Ronas Chemicals Ind. Co., Ltd., Chengdu, China. All other chemicals were of analytical grade and used without further purification.

Method 1 was used to obtain compounds of FORMULA 1. The roller ball mill was used to carry out mechanochemical synthesis of solid dispersions. Processing mode: rotational speed of the drive −400 rpm, rotational speed of the grinding jar −100 rpm, grinding jar volume −2000 ml, grinding media—steel balls (diameter 12 mm, 800 g/load), processing time ranging from 2 to 24 h. Samples are loose powders (particle size from 0.1-10 microns). Compositions and results of obtaining bioavailable solid dispersions with FORMULA I are shown in Table 2.

TABLE 2
Compounds of FORMULA I obtained in Example 1.

| No of | | | | HOST | | |
|---|---|---|---|---|---|---|
| FORMULA I | | | “API” | substance | | |
| compound | “API” | HOST substance | weight, g | weight, g | Ratio | Note |
| 1 | Ivermectin | Polyvinylpyrrolidone | 30 | 300 | 1:10 | |
| 2 | Ivermectin | apple pectin | 30 | 150 | 1:5 | |
| 3 | Ivermectin | Laminaria | 30 | 300 | 1:10 | forms a gel |
| | | | | | | with water |
| 4 | Ivermectin | SiO2 | 50 | 100 | 1:2 | silicon |
| | | | | | | dioxide |
| | | | | | | precipitates |
| | | | | | | when |
| | | | | | | dissolved |
| 5 | Ivermectin | hydroxyethyl starch | 50 | 500 | 1:10 | forms |
| | | | | | | clumps on |
| | | | | | | air |
| 6 | Ivermectin | carboxymethylcellulose | 50 | 150 | 1:3 | It takes a |
| | | sodium salt | | | | long time |
| | | | | | | to dissolve |
| | | | | | | in water |
| 7 | Ivermectin | Fucus | 50 | 350 | 1:7 | forms a gel |
| | | | | | | with water |
| 8 | Ivermectin | dioctyl sodium | 50 | 250 | 1:5 | |
| | | sulfosuccinate sodium | | | | |
| | | salt | | | | |

### Example 2

Method 10 was used to obtain compounds of FORMULA 1. Acetone or ethyl alcohol (rectified 96%) was used as organic solvents: 40-70 parts of the solvent per 100 parts of the polymer and avermectin mixture taken in gram-equivalent quantities. The named solvents dissolve the polymer host. After thoroughly stirring in a mortar the polymer with avermectin moistened with solvent, a dough-like mass is formed, which is dried with evaporation of the solvent. Ball mills or other grinders can be used for grinding the dried polymer mass, and the particle size of the polymer product should not exceed 5-9 microns. Compositions and results of obtaining bioavailable nanocomposites with FORMULA I are shown in Table 3.

TABLE 3
Compounds of FORMULA I obtained in Example 2.

| No of | | | | | | |
|---|---|---|---|---|---|---|
| FORMULA I | | | “API” | HOST substance | | |
| compound | “API” | HOST substance | weight, g | weight, g | Ratio | Solvent |
| 1 | Ivermectin | polyvinylpyrrolidone | 3 | 15 | 1:5 | ethanol |
| 2 | Ivermectin | apple pectin | 2 | 10 | 1:5 | ethanol |

### Example 3

Method 6 was used to obtain compounds of FORMULA 1. Ivermectin and PVA-polypropylene glycol graft co-polymers were used to create a solid dispersion. Hot stage extrusion was performed with a co-rotating, fully intermeshing conical mini twin screw extruder. The temperature was set at 195° C., the screw rate varied from 90 to 105 rpm. A load of 7.5 g per run was fed manually; after feeding the internal circulation time was 5 min. The extrudates were collected after cooling at ambient temperature on a conveyer belt. Extruded samples were subsequently milled for 4 min with a laboratory mill and sieved to exclude particles >400 pm.

### B. Bioavailability Study

### Example 4. Study of Ivermectin Concentration in the Blood Plasma of Cattle

Free ivermectin substance was processed in planetary mill, the parameters were as follows: weight of the material—5 g, grinding jar volume—200 mL, grinding media—steel balls (diameter 8 mm, 90.0 g load), processing time—20 minutes. Compound of FORMULA I was treated according to Example 1 N9 1.

Cattle were divided into 2 groups of 1 animals each. Free Ivermectin (IVM) and compound of FORMULA I were orally administered to starving cattle. Blood samples were withdrawn from the jugular vein at appropriate time intervals after administration (0, 4, 20 h).

Ivermectin concentrations in blood plasma were analyzed according to the method Na-Bangchang, K., High-performance liquid chromatographic method for the determination of ivermectin in plasma, Southeast Asian J Trop Med Public Health, 2006 September, 37(5):848-58, PMID: 17333725.

The internal standard (moxidectin) was separated from ivermectin on a Hypersil Gold C18 column (150×4.6 mm, 5 μm particle size), with retention time of 3.7 and 7.0 minutes, respectively. Fluorescence detection was set at an excitation and emission wavelength of 365 and 475 nm, respectively. The mobile phase consisted of acetonitrile, methanol and distilled water (50:45:5, v/v/v), running through the column at a flow rate of 1.5 ml/minute. The chromatographic analysis was operated at 25° C. Sample preparation (100 μl plasma) was done by a single stepprotein precipitation with acetonitrile, followed by derivatization with 100 μl of N-methylimidazole solution in acetonitrile (1:1, v/v) and 150 μl of trifluoroacetic anhydrous solution in acetonitrile (1:2, v/v). Calibration curve over the concentration range of 20-8,000 ng/ml plasma was linear with correlation coefficient better than 0.995.

All the blood samples were centrifuged at 3000 rpm for 15 min to obtain serum. Cattle plasma samples (100 μL each) were placed in 1.5 mL Eppendorf tubes, and then the samples underwent protein precipitation. The samples were vortexed for 1 min and centrifuged at 13,400 rpm for 10 min 20 μL of the supernatant was injected into the HPLC.

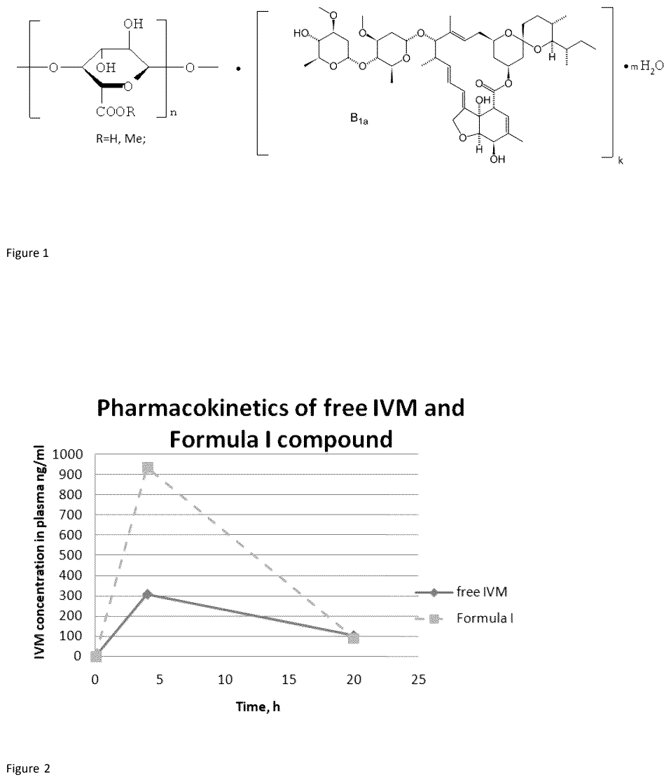

Pharmacokinetic parameters of Ivermectin and compound of FORMULA I are shown in FIG. 2 and in Table 4. The results provide evidence of a substantial increase in the bioavailability and permeability of compound of FORMULA 1. Oral administration of FORMULA I 600 μg/kg IVM results in an excess of EC 50 2.4 μM for SARS-CoV-2,

TABLE 4
Pharmacokinetic parameters (ivermectin concentrartion) after
oral administration of Ivermectin and compound of FORMULA I.

| | | compound of |
|---|---|---|
| | | FORMULA I of |
| | | enhanced |
| | | bioavailability |
| | | and permeability, |
| | IVM, | 600 μg/kg |
| | 600 μg/kg | (per pure IVM) |
| C1, 0 h, ng/ml | 0 ± 0.15 | 0 ± 0.15 |
| C2, 4 h, ng/ml | 307.3 ± 46.1 | 937.6 ± 140.6 |
| C3, 20 h, ng/ml | 104.4 ± 15.6 | 90.8 ± 13.6 |
| Cmax in plasma, μM | 0.35 ± 0.05 | 1.07 ± 0.16 |
| Cmax in lung, μM | 0.93 ± 0.14 | 2.86 ± 0.43 |
| calculated by | | |
| plasma-lung ratio | | |
| 2.67 | | |

### C. Permeability Study

### Example 5. Permeability of Solid Dispersion or the Noncovalent Complex on the Caco-2 Cell Line

The Caco-2 cell line is a standard in vivo model for evaluating the permeability of drugs through the gastrointestinal wall in vivo. These cells retain almost complete morpho-functional properties inherent to intestinal epithelial cells, including the expression of multidrug resistance proteins such as P-glycoprotein. As a rule, the data obtained in such experiments have a good correlation with the bioavailability of drugs in living organisms.

Caco-2 cells were cultured in a standard way in Dulbecco's medium with the addition of 10% thermally inactivated bovine serum (FBS, USA). The permeabilization study through the Caco-2 cell monolayer was performed according to standard procedures. Transwell 12-well plates with a 0.4 μm polyester membrane (Corning, USA) were used, and cells were seeded at a density of 1×105 cells per well. Before the experiment, cells were grown in IMDM medium supplemented with 10% FBS. The medium was changed every two days. The integrity of the cell monolayer was assessed by transepithelial electrical resistance (TEER) using Millicell ERS-2 (Millipore, USA) at the beginning and end of the experiment. The substance under study (compound of FORMULA I was treated according to Example 1 N9 2) was dissolved in Hanks' balanced salt solution (HBSS) at concentrations of 3 μM (per pure IVM). Ivermectin was similarly dissolved in Hanks' balanced salt solution at concentrations of 3 μM.

The test solutions were added to the top of the well, and the bottom of the well was filled with pure HBSS. The plates were then incubated at 37.5° C. and agitated (100 rpm) on an orbital shaker. Samples of 100 μL were taken from the bottom of the well at 20, 40, 60, 80, 100, and 120 min. After sampling, the required volume was immediately replenished with fresh HBSS solution. The concentration of substances was determined by high-performance liquid chromatography on Agilent 1200 HPLC with UV detector. The chromatographic analysis was performed on a reversed-phase column. The permeability coefficient was calculated according to a formula published in the literature.

TABLE 5
Permeability of IVM noncovalent complex through a monolayer
of Caco-2 cells compared to pure IVM at a similar point

| | | Concentrartion of IVM |
|---|---|---|
| | | (for compound of |
| | | FORMULA I |
| | Concentrartion | of enhanced |
| | of IVM (for | bioavailability |
| Time, | pure IVM), | and permeability), |
| min | μg/ml | μg/ml |
| 20 | 0.05 | 0.1 |
| 40 | 0.09 | 0.19 |
| 60 | 0.15 | 0.31 |
| 80 | 0.19 | 0.43 |
| 100 | 0.21 | 0.66 |
| 120 | 0.25 | 0.8 |

Thus, noncovalent ivermectin complexes have better permeability than pure ivermectin.

### D. Toxicity Study

### Example 6. Toxicity of Solid Dispersion or the Noncovalent Complex

Compound of FORMULA I was treated according to Example 1 N9 2. The oral toxicity (for Syrian hamsters) results of Compound of FORMULA I showed LD50=340 mg/kg (per pure ivermectin) and LD50=62 mg/kg for pure ivermectin, i.e. the toxicity of Compound I was reduced by 5.48 times.

### F. Spectroscopy Example 7. Spectroscopy of the Noncovalent Complex

Compound of FORMULA I was treated according to Example 1 N9 2. In the infrared spectrum of the obtained noncovalent complexes, the frequencies of the valence oscillations of the OH groups are observed as a broad shoulder, which in turn indicates the presence of intermolecular hydrogen bonds during complex formation. Based on infrared spectroscopy analysis, it was determined that noncovalent complexes are stabilized due to intermolecular hydrogen bonds.

To confirm the physicochemical values and chemical structure of the noncovalent complexes and to more accurately analyze the formation of the main molecular ion, the mass spectra of the complexes were analyzed by liquid chromatography-mass spectrometry on a TOF LC-MS (Agilent Technologies). Analysis of the mass spectra showed that, in addition to peaks belonging to individual carrier and API components, there are peaks corresponding to various composition of heterogeneous noncovalent structures. Ions corresponding to the molecular ions of the noncovalent complex are present in the mass spectrum, indicating that the carrier and API molecules formed noncovalent complexes due to mutual noncovalent interactions.

### G. Antiviral Activity

Another aspect of the invention relates to methods of inhibiting viral infections, comprising the step of treating a sample or subject suspected of needing such inhibition with a composition of the invention. Within the context of the invention samples suspected of containing a virus include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing an organism which induces a viral infection, frequently a pathogenic organism such as a tumor virus. Samples can be contained in any medium including water and organic solvent\water mixtures. Samples include living organisms such as humans, and man made materials such as cell cultures.

If desired, the anti-virus activity of a compound of the invention after application of the composition can be observed by any method including direct and indirect methods of detecting such activity. Quantitative, qualitative, and semiquantitative methods of determining such activity are all contemplated. Typically one of the screening methods described above are applied, however, any other method such as observation of the physiological properties of a living organism are also applicable.

The antiviral activity of a compound of the invention can be measured using standard screening protocols that are known. For example, the antiviral activity of a compound can be measured using the several general protocols.

### Example 8. Blood Oxygen Saturation Levels in Humans Study

The blood oxygen saturation levels of one symptomatic male patient and another symptomatic male patient who tested positive for SARS-CoV-2 was measured. Compound of FORMULA I was treated according to Example 1 No 3.

Humans were divided into 2 groups of 1 human in each. Compound of FORMULA I was administered orally 600 μg/kg (per pure ivermectin) to human in Group 2. Blood oxygen saturation levels were withdrawn at appropriate time intervals after administration (0, 24, 48, 72, 96 h).

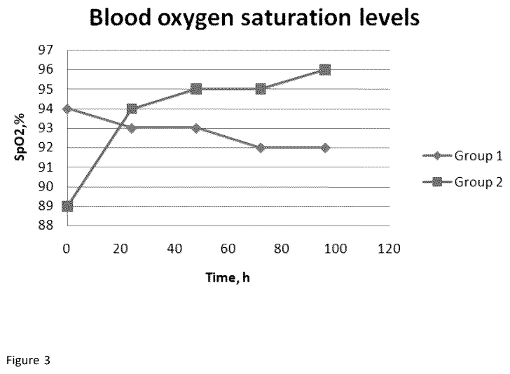

Changes in oxygen saturation levels for each study group on Day 0-4 are shown in Table 6 and FIG. 3. As shown in Table 6 and in FIG. 3, people who received compound FORMULA I showed a recovery in saturation levels associated with SARS-CoV-2 infection compared with those who did not receive treatment. As shown in FIG. 3, infectious virus was significantly reduced in people who received FORMULA I compound on days 1 and 2 after infection compared to untreated individuals. These data indicate that the FORMULA I compound reduces SARS-CoV-2 replication in the lungs.

TABLE 6
Blood oxygen saturation levels without treatment and
after oral administration of compound of FORMULA I.

| | | | Group 2, SpO2, % |
|---|---|---|---|
| | | | after oral |
| | | | administration |
| | | Group 1, | compound of |
| | | SpO2, %, | FORMULA I, |
| | | without | 600 μg/kg (per |
| | | treatment | pure ivermectin) |
| 0 | h | 94 | 89 |
| 24 | h | 93 | 94 |
| 48 | h | 93 | 95 |
| 72 | h | 92 | 95 |
| 96 | h | 92 | 96 |

### Example 9. SARS-CoV-2 Viral Load in Humans Study

The viral load of a symptomatic male patient who tested positive for SARS-CoV-2 was measured. Compound of FORMULA I was treated according to Example 2 No 2.

The SARS-CoV-2 viral load from nasopharyngeal swabs was quantified from samples stored at −40° C. until use. Viral RNA was isolated using the QIAamp Viral RNA Mini Kit (Qiagen, Hilden, Germany) from stored samples.

Quantitative reverse transcriptase PCR (RT-qPCR) targeting the SARS-CoV-2 N-gene was then performed. The standard curve consisted of in vitro transcribed viral RNA serially diluted in a matrix of cellular RNA from nasopharyngeal negative samples.

This assay included measurement of the housekeeping gene as an internal control and normalizer. The cycle threshold (Ct) of the housekeeping gene was used to correct the specific SARS-CoV-2 Ct according to the number of cells in the sample. Therefore, viral load measurements were expressed in log 10 copies per reaction.

The log 10 viral load before oral administration of FORMULA I compound was 6.3 copies per mL, and log 10 viral load 24 hours after administration 1200 ag/kg (per pure ivermectin) of FORMULA I was 5.1 copies per mL. The log 10 viral load was significantly different between the start of treatment (log 10 6.3 copies per mL), and 24 hours after the start of treatment (log 10 5.1 copies per mL). These data suggest that compound of FORMULA I reduces replication of SARS-CoV-2.

TABLE 7
Viral load levels before and 24 h after oral administration
of compound of FORMULA I 1200 μg/kg.

| | Patient 1, log10 copies per mL, |
|---|---|
| | compound of FORMULA I, 1200 |
| | μg/kg (per pure ivermectin) |
| 0 h | 6.3 |
| 24 h | 5.1 |

### Example 10. Patient Study

Dengue, chikungunya, yellow fever and Zika fever are common in the tropics, South and Central America, Mexico (including the states of Veracruz, Jalisco, Chiapas, Oaxaca and Quintana Roo) and the Caribbean. The virus is transmitted by female Aedes aegypti and Aedes albopictus mosquitoes.

A 38-year-old man tested positive for NS1 test, which detects the nonstructural NS1 protein of the dengue virus. Symptoms include fever of 40° C./104° F., severe headache, nausea, muscle aches. The patient received the compound of FORMULA I (obtained according to Example 1 No. 2) as a mixture with 300 ml water of 800 ag/kg per pure ivermectin. The compound is administered orally. The patient's fever gradually decreases, muscle pain recovers, and after another 2 days the symptoms of the disease disappear.

### VIII. CONCLUDING REMARKS

Names of compounds of the present disclosure are provided using ACD/Name software for naming chemical compounds (Advanced Chemistry Development, Inc., Toronto, Canada). Other compounds or radicals may be named with common names or systematic or non-systematic names. The names of internationally registered chemical compounds and pharmaceutical preparations may be given in English. This applies to recognized names such as INN (International Nonproprietary Names)—see WHO International Nonproprietary Names Programme and Classification of Medical Products (https://www.who.int/teams/health-product-and-policy-standards/inn), as well as IUPAC (International Union of Pure and Applied Chemistry) names.

When trade names are used herein, applicants intend to independently include the trade name product and the API(s) of the trade name product.

The compounds of the present invention can be prepared by methods known to one of skill in the art.

The invention has been described with reference to various specific and preferred embodiments and techniques. However, one skilled in the art will understand that many variations and modifications may be made while remaining within the spirit and scope of the invention.

While the present invention has been described in detail and with reference to specific embodiments thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents. The reference to weight percents includes the weight percent of the component in the completed formula, unless noted otherwise.

Although the disclosed inventions are illustrated and described herein as embodied in one or more specific examples, it is nevertheless not intended to be limited to the details shown, since various modifications and structural changes may be made therein without departing from the scope of the inventions. In addition, various features from one of the embodiments may be incorporated into another of the embodiments. Accordingly, it is appropriate that the invention be construed broadly and in a manner consistent with the scope of the disclosure.

Having described preferred embodiments, it is believed that other modifications, variations and changes will be suggested to those skilled in the art in view of the teachings set forth herein. It is therefore to be understood that all such variations, modifications and changes are believed to fall within the scope of the present invention as defined by the appended claims. Although specific terms are employed herein, they are used in a generic and descriptive sense only and not for purposes of limitation.

1. A non-covalent complex comprising a pharmaceutically active ingredient (API, GUEST) (selected from the group consisting of ivermectin or avermectin) and a HOST substance, wherein:
A) The API is ivermectin Bla, and the HOST substance is pectin, forming a complex represented by the following structure: 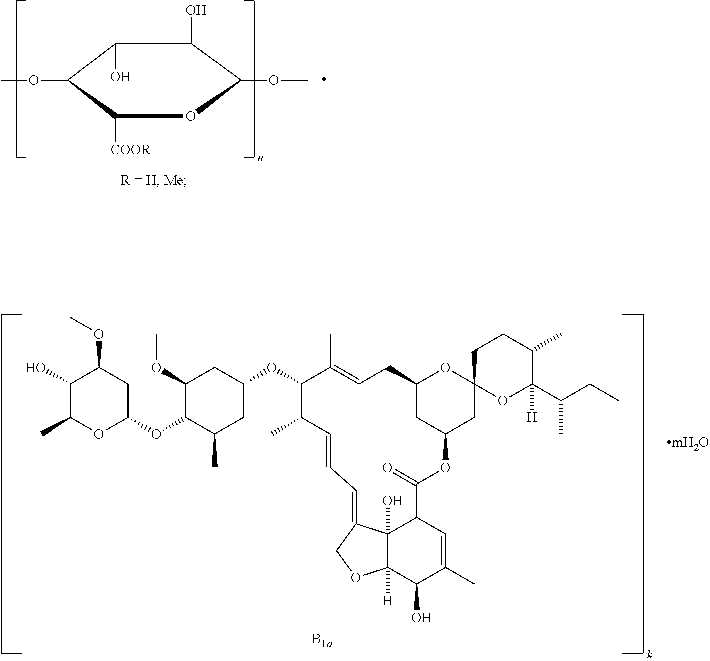
B) The API is ivermectin B1b, and the HOST substance is pectin forming a complex represented by the following structure: 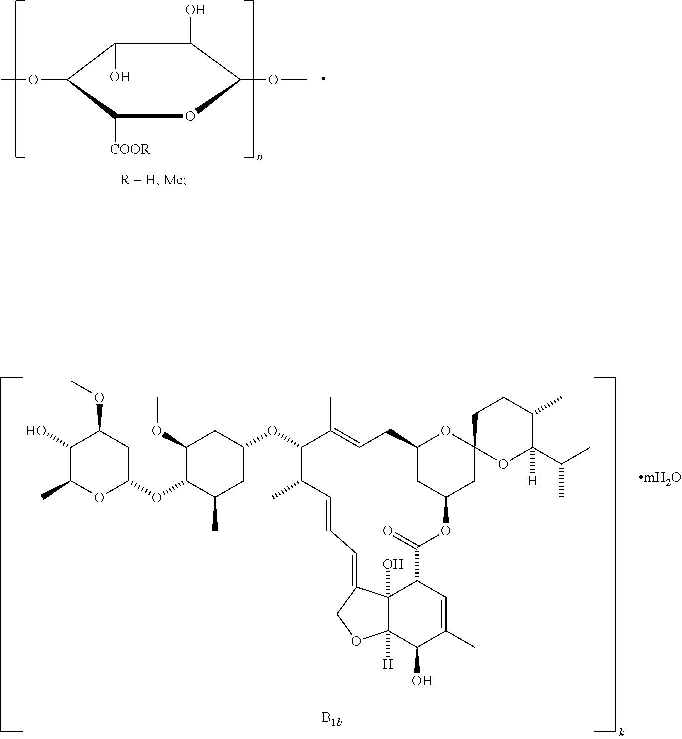
C) The HOST substance is pectin, and the API is selected from the group consisting of ivermectins and avermectins (A1a, A1b, A2a, A2b, Bla, B1b, B2a, B2b), including any racemate, enantiomer, diastereomer, tautomer, polymorph, pseudopolymorph, hydride, solvate, pharmaceutically acceptable salt, ester, prodrug, or mixture or derivative thereof,
forming a complex represented by the following structure: 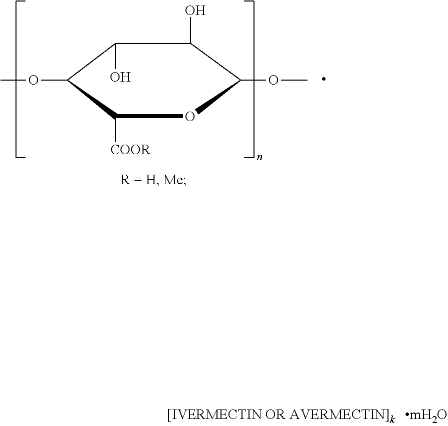
and the formula of the complex can be established by the specialist on the basis of the InChlKey provided;
wherein k is 1 to 100; wherein m is 1 to 10000; wherein n is 1 to 10000;
for use in manufacturing a pharmaceutical dosage form to treat viral infections in a human in need thereof (by administering a therapeutically effective amount) or use in manufacturing or preparing a medical dosage form used as an antiviral agent,
at a dosage ranging from 10 ag/kg to 100 mg/kg (calculated for pure ivermectin or pure avermectin),
wherein said dosage provides improved therapeutic efficacy (for example, achieving treatment within 10 to 48 hours for certain viral infections, where known methods do not reduce the duration of the disease, and treatment typically takes more than 12 days);
wherein the non-covalent complex has enhanced permeability (into the internal organs and tissues of the person receiving the drug) and bioavailability properties;
and wherein the therapeutic efficacy of the non-covalent complex is enhanced by enhanced permeability, which allows for rapid achievement of the required concentration in the organs and tissues of the patient.

2. A non-covalent complex comprising a pharmaceutically active ingredient (API, GUEST) (selected from the group consisting of ivermectin or avermectin) and a HOST substance, wherein:
A) The API is ivermectin Bla, and the HOST substance is arabinogalactan, forming a complex represented by the following structure: 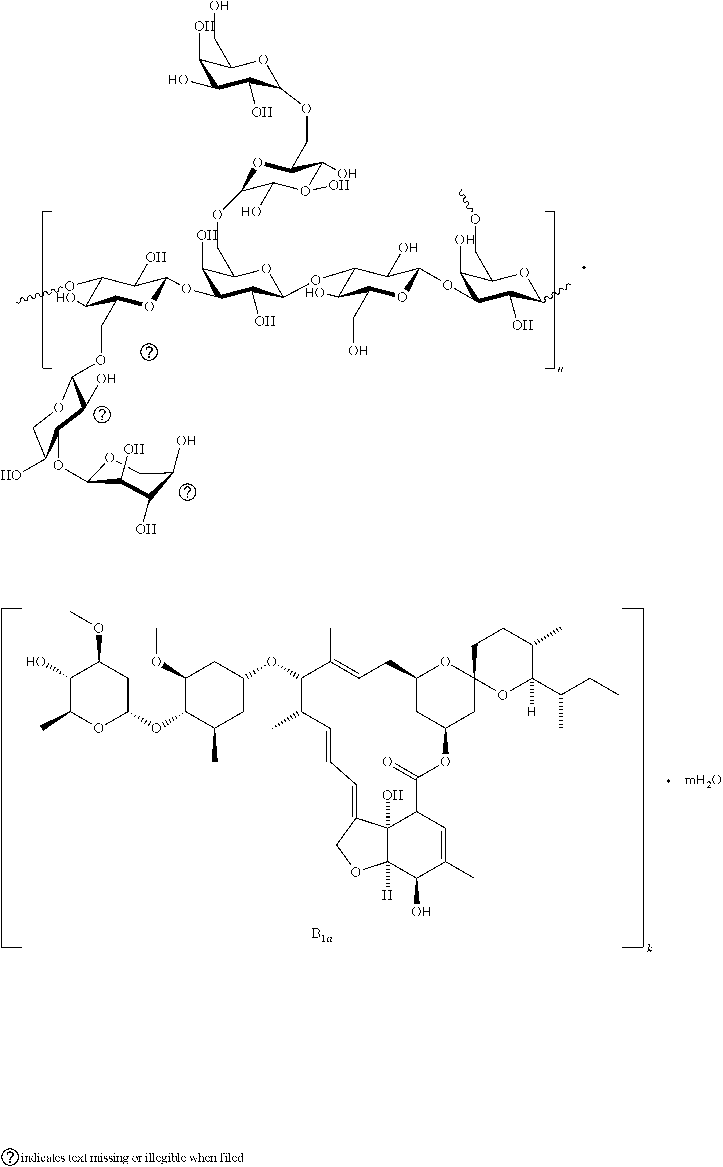
B) The API is ivermectin B1b, and the HOST substance is arabinogalactan, forming a complex represented by the following structure: 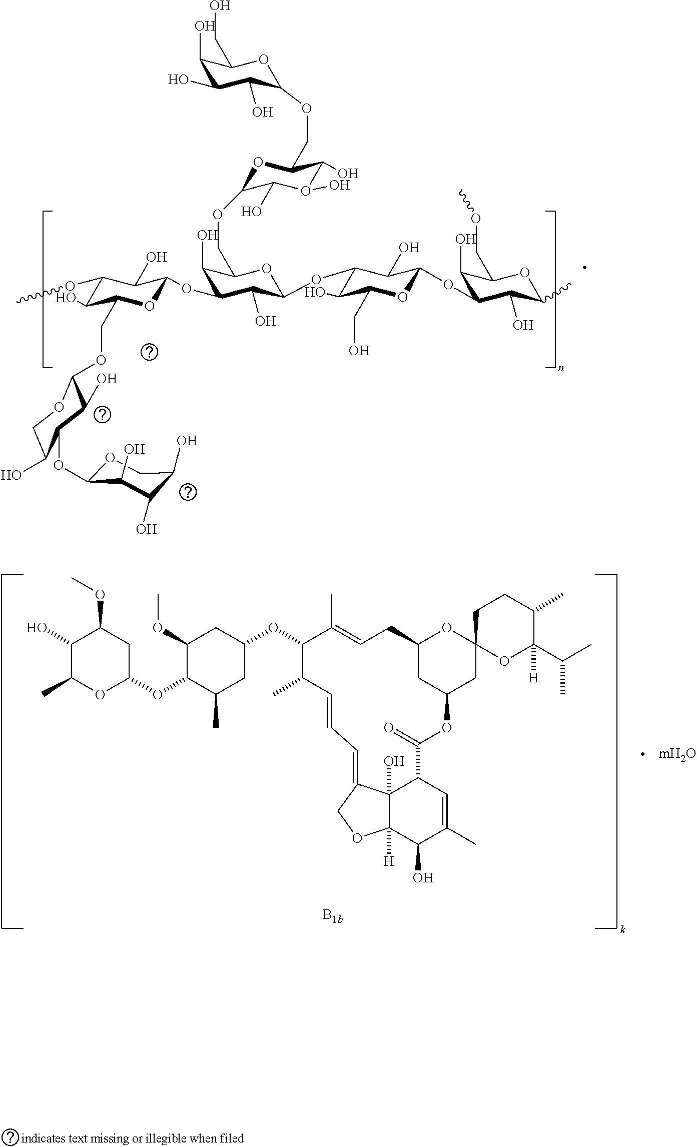
C) The HOST substance is arabinogalactan, and the API is selected from the group consisting of ivermectins and avermectins (A1a, A1b, A2a, A2b, Bla, B1b, B2a, B2b), including any racemate, enantiomer, diastereomer, tautomer, polymorph, pseudopolymorph, hydride, solvate, pharmaceutically acceptable salt, ester, prodrug, or mixture or derivative thereof, and the formula of the complex can be established by the specialist on the basis of the InChlKey provided;
D) The API is ivermectin Bla, and the HOST substance is laminarin, forming a complex represented by the following structure: 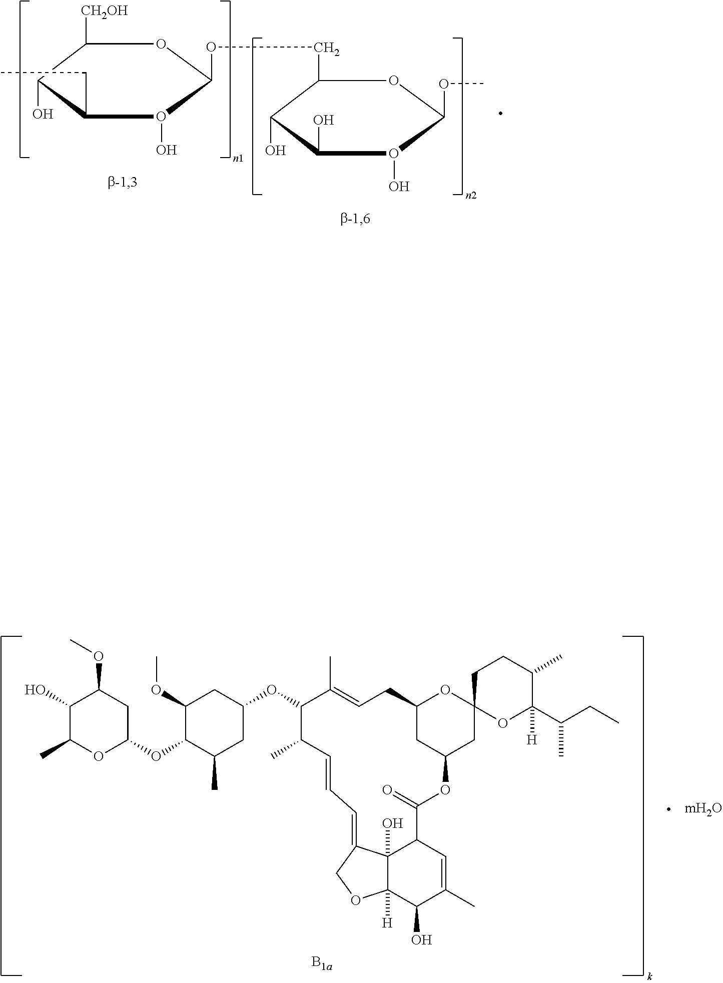
E) The API is ivermectin B1b, and the HOST substance is laminarin, forming a complex represented by the following structure: 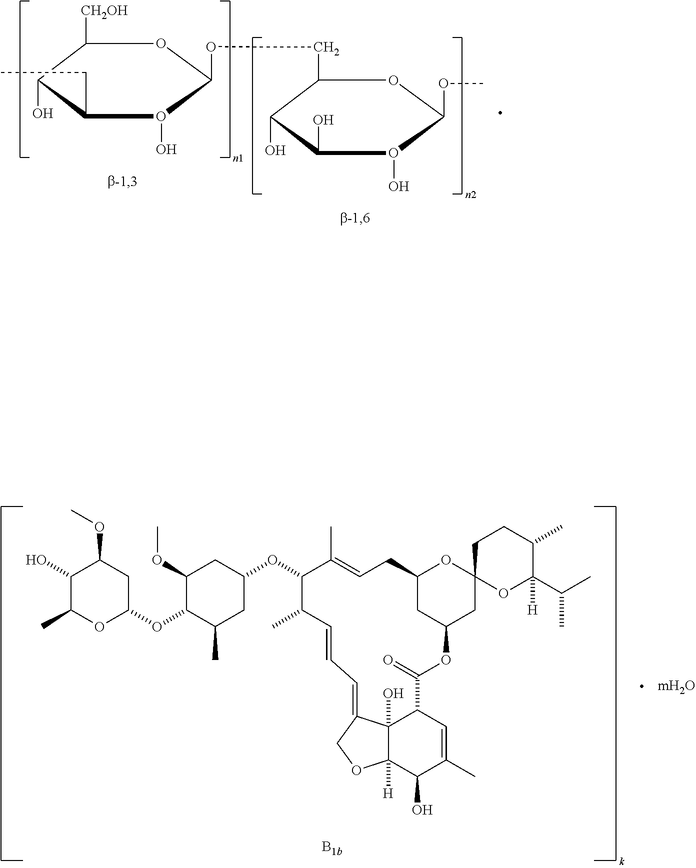
F) The HOST substance is laminarin, and the API is selected from the group consisting of ivermectins and avermectins (A1a, A1b, A2a, A2b, Bla, B1b, B2a, B2b), including any racemate, enantiomer, diastereomer, tautomer, polymorph, pseudopolymorph, hydride, solvate, pharmaceutically acceptable salt, ester, prodrug, or mixture or derivative thereof, and the formula of the complex can be established by the specialist on the basis of the InChlKey provided;
G) The API is ivermectin Bla, and the HOST substance is polyvinylpyrrolidone, forming a complex represented by the following structure: 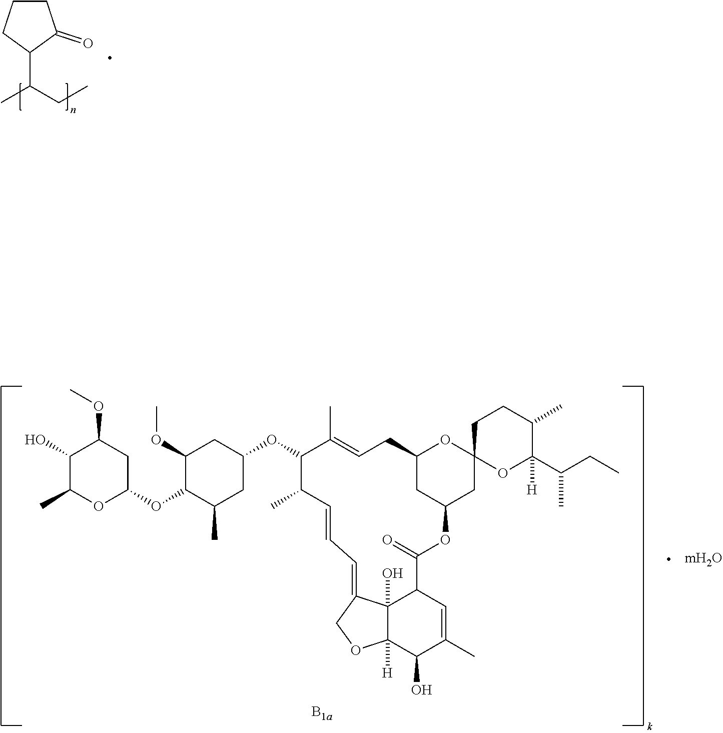
H) The API is ivermectin B1b, and the HOST substance is polyvinylpyrrolidone, forming a complex represented by the following structure: 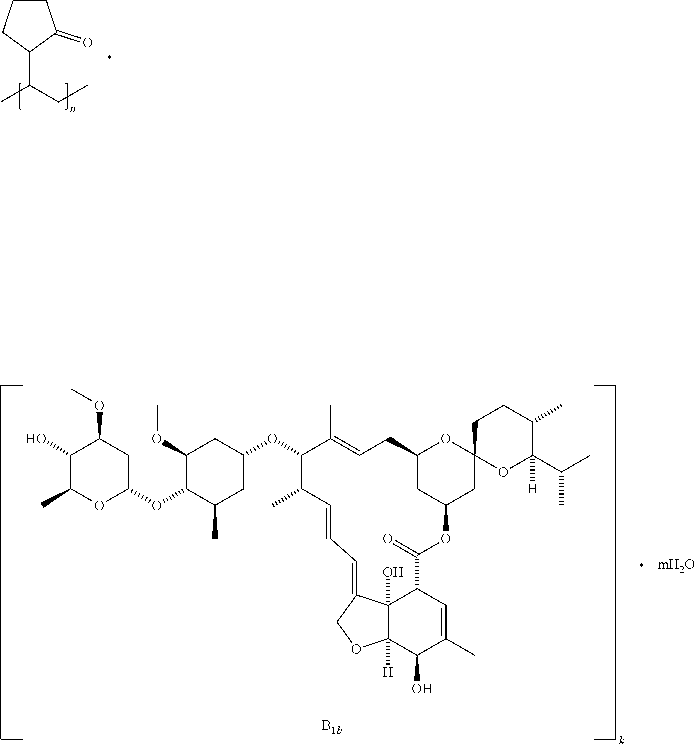
I) The HOST substance is polyvinylpyrrolidone, and the API is selected from the group consisting of ivermectins and avermectins (A1a, A1b, A2a, A2b, Bla, B1b, B2a, B2b), including any racemate, enantiomer, diastereomer, tautomer, polymorph, pseudopolymorph, hydride, solvate, pharmaceutically acceptable salt, ester, prodrug, or mixture or derivative thereof, and the formula of the complex can be established by the specialist on the basis of the InChlKey provided;
J) The API is ivermectin Bla, and the HOST substance is glycyrrhizic acid, forming a complex represented by the following structure: 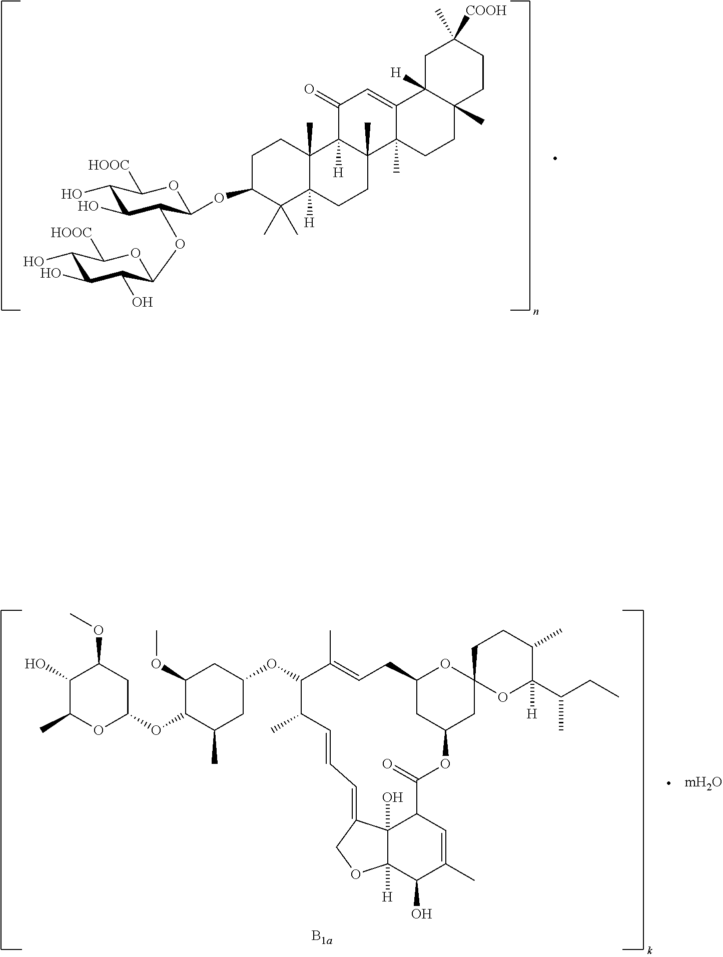
K) The API is ivermectin B1b, and the HOST substance is glycyrrhizic acid forming a complex represented by the following structure: 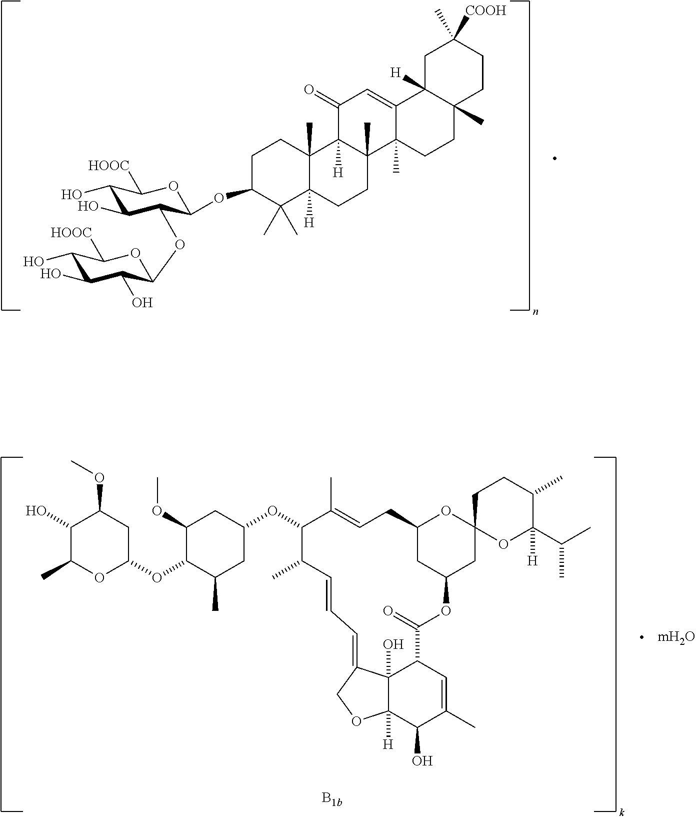
L) The HOST substance is glycyrrhizic acid, and the API is selected from the group consisting of ivermectins and avermectins (A1a, A1b, A2a, A2b, Bla, B1b, B2a, B2b), including any racemate, enantiomer, diastereomer, tautomer, polymorph, pseudopolymorph, hydride, solvate, pharmaceutically acceptable salt, ester, prodrug, or mixture or derivative thereof, and the formula of the complex can be established by the specialist on the basis of the InChlKey provided;
wherein k is 1 to 100; wherein m is 1 to 10000; wherein n is 1 to 10000; wherein n1, n2 is 1 to 10000;
for use in manufacturing a pharmaceutical dosage form to treat viral infections in a human in need thereof (by administering a therapeutically effective amount) or use in manufacturing or preparing a medical dosage form used as an antiviral agent,
at a dosage ranging from 10 ag/kg to 100 mg/kg (calculated for pure ivermectin or pure avermectin),
wherein said dosage provides improved therapeutic efficacy (for example, achieving treatment within 10 to 48 hours for certain viral infections, where known methods do not reduce the duration of the disease, and treatment typically takes more than 12 days);
wherein the non-covalent complex has enhanced permeability (into the internal organs and tissues of the person receiving the drug) and bioavailability properties;
and wherein the therapeutic efficacy of the non-covalent complex is enhanced by enhanced permeability, which allows for rapid achievement of the required concentration in the organs and tissues of the patient.

3. Methods (co-treating methods/solid dispersion techniques) for modifying the properties of a compound (ivermectin, avermectin) by creating a molecular/solid dispersion (compound of FORMULA I, using as precursors ivermectin or avermectin (API, GUEST), HOST substance and, if necessary, other compounds) with properties of increased bioavailability and permeability (into the internal organs and tissues of the person receiving the drug)
and using compounds obtained by this methods (or aqueous solutions or suspensions thereof) to manufacture a pharmaceutical dosage form for the treatment of viral infections in a human in need thereof (by administering a therapeutically effective amount), or for use in manufacturing or preparing a medical dosage form used as an antiviral agent,
at a dosage ranging from 50 ag/kg to 250 mg/kg (calculated for pure ivermectin or pure avermectin)
wherein said dosage provides improved therapeutic efficacy (for example, achieving treatment within 10 to 48 hours for certain viral infections, where known methods do not reduce the duration of the disease, and treatment typically takes more than 12 days);
wherein the non-covalent complex has enhanced permeability (into the internal organs and tissues of the person receiving the drug) and bioavailability properties;
and wherein the therapeutic efficacy of the non-covalent complex is enhanced by enhanced permeability, which allows for rapid achievement of the required concentration in the organs and tissues of the patient;
wherein the ivermectin, avermectin (API, GUEST) is selected from:
ivermectins and avermectins (A1a, A1b, A2a, A2b, Bla, B1b, B2a, B2b) or racemate, enantiomer, diastereomer, tautomer, polymorph, pseudopolymorph, hydride or solvate thereof or a pharmaceutically acceptable salt or ester thereof or prodrug thereof or mixtures thereof or derivatives thereof;
wherein the HOST substance is selected from:
A. Polymers and oligomers, predominantly organic polymers and oligomers, even more predominantly polysaccharides and oligosaccharides, hemicelluloses, storage polysaccharides, sulfated polysaccharides and oligosaccharides, pectins, gums, mucilages, which may be (but are not exclusive to):
A.1. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, their mixtures, but not excluding other hemicelluloses, and mixtures thereof;
A.2. Sulfated polysaccharides and oligosaccharides which may be fucoidans, carrageenans or carrageenins, agaropectins, sea cucumber sulfated polysaccharides (SCSP), chondroitin sulfate, keratan sulfate, their mixtures, but not excluding other sulfated polysaccharides and oligosaccharides, and mixtures thereof;
A.3. Polysaccharides and oligosaccharides, storage polysaccharides, sulfated polysaccharides and oligosaccharides, pectins, gums, mucilages which may be (but are not exclusive to others): polysaccharides based on glucose, dextrose, galactose, mannose, arabinose, rhamnose, sucrose, maltose, lactose and their uronic acids, which may be methoxylated or acetylated, and their salts (gum, gummi); polyuronic acids and esters; gum, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups, phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses, galacto-oligosaccharides (oligogalactosyllactose, oligogalactose, oligolactose or transgalactooligosaccharides), xylooligosaccharides, fructooligosaccharides, isomaltooligosaccharide; alginic acid and its salts alginates, propylene glycol alginate; arabin, arabic acid and its salts; cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s) (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch; dextrin(s), maltodextrins, cyclodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid and mixtures thereof, but not excluding other polysaccharides and oligosaccharides, hemicelluloses, stored polysaccharides, sulfated polysaccharides and oligosaccharides, mucilages and mixtures thereof;
A.4. Macrocyclic hosts which may be (but are not exclusive to others): cyclodextrins, cucurbit[n]urils, and calix[n]arenes, pillarenes, crown ethers, cyclophanes, cryptands;
B. Substances that may contain in significant amounts (more than 5% wt.) polysaccharides and oligosaccharides, hemicelluloses, storage polysaccharides, sulfated polysaccharides and oligosaccharides, pectins, gums, mucilages, which may be (but are not limited to) plants or algae or animal or fungi, parts of plants or algae or animal or fungi, processed plants or algae or animal or fungi, processed parts of plants or algae or animal or fungi containing in significant amounts the substances specified in paragraph A, and mixtures thereof. A frequent but non-limiting example is dried brown or red algae such as kelp or focus;
C. Syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s, poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit S100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s), Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone 5-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-α-tocopherol polyethylene glycol 1000 succinate (TPGS), Isomalt (Galen IQ 810), but not excluding other synthetic polymers and mixtures thereof;
D. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
E. Saccharides which may be glucose, dextrose, galactose, mannose, arabinose, rhamnose, sucrose, maltose, lactose, ribose, and mixtures thereof;
F. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
G. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof;
H. Oxides and salts based on these oxides which may be (but are not limited to): silicon dioxide, silicates, titanium dioxide, and mixtures thereof;
I. Copolymers of the polymers listed in paragraph A, C including alternating copolymers, random copolymers, block copolymers, graft copolymers, cross-linked modifications which may be (but are not limited to): methacrylic acid and ethyl acrylate copolymers; methacrylic acid copolymers; vinylpyrrolidone-vinyl acetate copolymers (PVPVA); polyvinylpolypyrrolidone (PVPP); HPMC and PVA-PEG grafted copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols orpolymeth-ylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral copolymers based on methacrylic acid and methacrylic/acrylic esters their salts, esters or other derivatives and mixtures thereof;
J. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-I, and their esters, salts, and any other chemical derivatives, and mixtures thereof;
K. Any combination of substances specified in items A-J;
but excluding Kollicoat IR and similar PVA-PEG graft copolymers;
wherein co-treating method (solid dispersion preparation method) is selected from:
a) grinding/milling with high energy stress method;
b) grinding/milling with high energy stress and solvent method;
c) media milling method;
d) kneading method;
e) hot-melt method/melting method/fusion method;
f) hot-melt extrusion/hot-stage extrusion method;
g) meltrex method;
h) melt agglomeration method;
i) high-pressure homogenization method;
j) solvent evaporation method;
k) spin-coated films method;
l) spray-drying method;
m) supercritical fluid (SCF) process method;
n) cryogenic techniques method;
o) lyophilization/freeze-drying technique method;
p) spray freezing onto cryogenic fluids method;
q) spray freezing into cryogenic liquids (SFL) method;
r) spray freezing into vapor over liquid (SFV/L) method;
s) ultra-rapid freezing method;
t) precipitation/co-precipitation method;
u) microwave irradiation method;
v) energy input method;
w) heat/shear energy input method;
x) a method comprising: contacting an active pharmaceutical ingredient and a matrix (HOST substance) to form a solid dispersion under conditions sufficient to form a solid dispersion;
y) combined method.

4. Non-covalent complex of ivermectins, avermectins (API, GUEST) and HOST substance with properties of increased permeability and bioavailability (into the internal organs and tissues of the person receiving the drug) for manufacturing a pharmaceutical dosage form for treat of viral infections in a human in need thereof (by administering a therapeutically effective amount),
or for use in manufacturing or preparing a medical dosage form used as an antiviral agent,
in a dosage from 10 ag/kg to 100000 ag/kg (calculated for pure ivermectin or pure avermectin),
wherein said dosage provides improved therapeutic efficacy (for example, achieving treatment within 10 to 48 hours for certain viral infections, where known methods do not reduce the duration of the disease, and treatment typically takes more than 12 days);
wherein the non-covalent complex has enhanced permeability (into the internal organs and tissues of the person receiving the drug) and bioavailability properties;
and wherein the therapeutic efficacy of the non-covalent complex is enhanced by enhanced permeability, which allows for rapid achievement of the required concentration in the organs and tissues of the patient.
wherein the GUEST is selected from:
ivermectins and avermectins (A1a, A1b, A2a, A2b, Bla, B1b, B2a, B2b) or racemate, enantiomer, diastereomer, tautomer, polymorph, pseudopolymorph, hydride or solvate thereof or a pharmaceutically acceptable salt or ester thereof or prodrug thereof or mixtures thereof or derivatives thereof;
wherein the molecular information for the non-covalent complex of FORMULA II is encoded by the formula (the non-covalent complex of FORMULA II is):
[GUEST]ₖ [HOST]ₙ (SOLVENT)ₘ
wherein k is 1 to 100; wherein m is 1 to 10000; wherein n is 1 to 10000;
where GUEST molecular information is given based on the InChl derived from the InChlKey indicated for each of the GUEST drug in parentheses after the name of the guest drug compound, in the second position after the sequence numbers (in paragraph [0061](III.I. Section)). The molecular information of guest drug compounds can be used on its own as well as on the basis of it (by an skilled in the art) the molecular informations can be obtained for pharmaceutical salts, esters, ethers, free bases and other pharmaceutical salts of these free bases;
wherein HOST molecular information represents the molecular information of a monomer of a “HOST substance” polymer; or the smallest representable part of a “HOST substance” copolymer (defining that copolymer); or “HOST substance” molecule (if HOST substance is in monomolecular form) for compounds that are selected from the HOST substances and can be obtained by an skilled in the art;
wherein SOLVENT molecular information is H₂O if solvent is water, but other physiologically acceptable solvents can also be used and the molecular information can be obtained by an skilled in the art;
or the formula of the complex can be established by the specialist (by an skilled in the art);
wherein the HOST substance is selected from:
A. Polymers and oligomers, predominantly organic polymers and oligomers, even more predominantly polysaccharides and oligosaccharides, hemicelluloses, storage polysaccharides, sulfated polysaccharides and oligosaccharides, pectins, gums, mucilages, which may be (but are not exclusive to):
A.1. Hemicelluloses, which can be arabanes, arabinans, galactans (galactosans), glucans, xylans, mannans, fructans, xyloglucans, arabinogalactans, arabinoxylans, glucomannans, galactomannans, galactoglucomannans, beta-glucans, galactogens, their mixtures, but not excluding other hemicelluloses, and mixtures thereof;
A.2. Sulfated polysaccharides and oligosaccharides which may be fucoidans, carrageenans or carrageenins, agaropectins, sea cucumber sulfated polysaccharides (SCSP), chondroitin sulfate, keratan sulfate, their mixtures, but not excluding other sulfated polysaccharides and oligosaccharides, and mixtures thereof;
A.3. Polysaccharides and oligosaccharides, storage polysaccharides, sulfated polysaccharides and oligosaccharides, pectins, gums, mucilages which may be (but are not exclusive to others): polysaccharides based on glucose, dextrose, galactose, mannose, arabinose, rhamnose, sucrose, maltose, lactose and their uronic acids, which may be methoxylated or acetylated, and their salts (gum, gummi); polyuronic acids and esters; gum, xanthan gum, oat gum, gellan gum, guar gum, carob gum, karaya gum, dammar gum, gummiarabica, tara gum, ghatti gum, british gum, agar, agar-agar, tragakant gum, conjac gum, velan gum, dutan gum; galacturonic acid-based polysaccharides with side chains of rhamnose, arabinose, xylose and fructose and their salts (pectins, pectates, pectinates); pectin, pectins of beets, carrots, peppers, pumpkins, eggplant, sunflower, apples, quinces, cherries, plums, pears, citrus, zosterin; modified pectins, modified citrus pectin; acidic polysaccharides—i.e. e. polysaccharides containing carboxyl groups, phosphate groups and/or sulfuric ester groups; plantain husk, psyllium; soybean hemicellulose; galacturones, homogalacturones, polygalacturonic acids and their salts, rhamnogalacturonan, rhamnogalactans; calloses, laminarins, chrysolaminarins, curdlans; inulins, guars, dextrans, pullulans; agaroses, galacto-oligosaccharides (oligogalactosyllactose, oligogalactose, oligolactose or transgalactooligosaccharides), xylooligosaccharides, fructooligosaccharides, isomaltooligosaccharide; alginic acid and its salts alginates, propylene glycol alginate; arabin, arabic acid and its salts; cellulose, cellulosic polymers, methylcellulose, ethylcellulose, hydroxypropylcellulose(s) (HPC), hydroxypropylmethylcellulose acetate succinate, hypromellose(s), hydroxypropylmethylcellulose(s) (HPMC), methylethylcellulose, ethylhydroxyethylcellulose, croscaramellose, carboxymethylcellulose and its salts; starch, starch(es), starch 1500G, soluble starch, modified starches, hydroxyethyl starch, cationic starch, acid-treated starch, alkaline modified starch, bleached starch, oxidized starch, enzyme treated starch, monostarch phosphate, distarch glycerol, distarch phosphate, phosphated distarch phosphate, acetylated distarch phosphate, starch acetate esterified with acetic anhydride, starch acetate esterified with vinyl acetate, acetylated distarch adipate, acetylated distarch glycerol, distarch glycerine, hydroxy propyl starch, hydroxy propyl distarch glycerine, hydroxy propyl distarch phosphate, hydroxy propyl distarch glycerol, starch sodium octenyl succinate, acetylated oxidised starch; dextrin(s), maltodextrins, cyclodextrins, amylodextrins, polydextroses; amylopectin, amylose, glycogen; chitosan, chitins; pullulans, glucuronoaraboxylans, methyl-glucuronoaraboxylans, glycosaminoglycans, mucopolysaccharides, heparin/heparan sulfate, chondroitin sulfate, dermatan sulfate, keratan sulfate, hyaluronan, hyaluronic acid and mixtures thereof, but not excluding other polysaccharides and oligosaccharides, hemicelluloses, stored polysaccharides, sulfated polysaccharides and oligosaccharides, mucilages and mixtures thereof;
A.4. Macrocyclic hosts which may be (but are not exclusive to others): cyclodextrins, cucurbit[n]urils, and calix[n]arenes, pillarenes, crown ethers, cyclophanes, cryptands;
B. Syntetic polymers, predominantly water-soluble polymers which may be (but are not limited to): polymers and copolymers formed from acrylic acid, methacrylic acid, and/or esters thereof; polymethacrylates, Eudragit and their salts; polyacrylamides; poly(amidoamine)s, poly(propyleneimine)s, polyethylene glycols; polyvinyl alcohol; polyvinylpyrrolidone; acrylic polymers, cellulose acetate phthalate(s), copovidone(s), ethyl oleate, glycerol derivatives, glyceryl triacetate, polyethylene glycol(s) (PEG), PEG derivatives, polymethacrylates, propylene glycol, propylene glycol derivatives, povidone(s), polyvinylpyrrolidone(s) (PVP), polyvinyl acetate phthalate(s) (PVAP), hypromellose acetate succinate(s) (HPMCAS), hydroxypropyl methylcellulose acetate succinate (HPMCAS), hypromellose phthalate(s) (HPMCP), cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, Avicel(s), Avicel PH101, Avicel PH102, Benecel(s), Brij(s), Brij 30, Brij 35, Capryol(s), Cavamax(s), Cavasol(s) and Cavitron(s) HPpCD cyclodextrins, Compritol 888 ATO, Cremophor(s), Cremophor EL, Cremophor RH40, DiCal Dihydrate, epoxidized palm oil (Epo), Eudragit(s), Eudragit E, Eudragit EPO, Eudragit L100, Eudragit L100-55, Eudragit S100, Gelucire(s), Gelucire 44/14, HP-50 AAS-LF, HP-55 AAS-MF, HPMC(p-606), HPMC-E, HPMC-F, HPMC-K, HPMCAS-H, HPMCAS-L, HPMCAS-M, HPMCAS SDD, HPMCAS(AS-MG), HPMCAS-M SDDs, HPMCAS-MG, HPMCP (HP 55), HPMCP-HP55, HPMCPh, hypromellose phthalate HP-50, Imwitor(s), Imwitor 742, Klucel HPC, Kolhdon 17 PF, Kollicoat MAE, Kollicoat MAE 100, Kollicoat MAE 100P, Kollicoat Protect, Kollidon(s) (povidone(s)), Kollidon 12 pf, Kollidon 12/17PF, Kollidon 30, Kollidon 30/90, Kollidon 90, Kollidon CL-F, Kollidon CL-SF, Kollidon K30, Kollidon SR, Kollidon SR, Kollidon SR(PVAc), Kollidon V64/Fine, Kollidon VA 64, Kollidon VA 64 (copovidone), Kollidon VA64, Kolliphor(s), Kolliphor EL, Kolliphor EL/ELP, Kolliphor HS15, Kolliphor P 188, Kolliphor P 188/407, Kolliphor P 188/micro, Kolliphor P 407, Kolliphor P 407/micro, Kolliphor PS 20, Kolliphor PS 60, Kolliphor PS 80, Kolliphor RH 40, Kolliphor SLS, Kolliphor SLS/fine, Kollisolv(s), Kollisolv GTA, Kollisolv PEG 1450, Kollisolv PEG 300, Kollisolv PEG 3350, Kollisolv PEG 400, Kollisolv PEG E 300, Kollisolv PEG E 400, Kollisolv PEG grades (polyethylene glycol), Kolliwax(s), Kolliwax GMS II, Kolliwax SA, Labrasol(s), Lactose 310 Mono, Lactose FF316, Laurogucol(s), Maisine(s),
Miglyol(s), Myrj(s), Myrj 52, PEG 1000, PEG 10000, PEG 1500, PEG 2000, PEG 20000, PEG 3000, PEG 400, PEG 4000, PEG 600, PEG 6000, PEG 800, PEG 8000, Pharmacoat(s), PVP K-12, PVP K-120, PVP K-15, PVP K-17, PVP K-30, PVP K-60, PVP K-90, PVP SDD, PVP VA64 SDDs, PVP-VA, PVP-VA 64, PVP-VA SDD, Palm stearin based polyesteramide (PSPEA), Peceol(s), pectin(s), Plasdone(s), Plasdone K povidone, Plasdone K-12 povidone, Plasdone K-29/32 povidone, Plasdone K-90 povidone, Plasdone S, Plasdone S-630 copovidone, poly(2-ethyl-2-oxazoline), poly(ethylene oxide) (PEO) (3400, 10000, 20000), polyoxyethylene stearate, Shin-Etsu AQOAT(s), Soluplus(es), Solutol(s), sucrose laurate, tocopheryl PEG 1000-succinate (TPGS), vitamin E TPGS, d-α-tocopherol polyethylene glycol 1000 succinate (TPGS), Isomalt (Galen IQ 810), but not excluding other synthetic polymers and mixtures thereof;
C. Polyols which may be (but are not limited to): ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and mixtures thereof;
D. Saccharides which may be glucose, dextrose, galactose, mannose, arabinose, rhamnose, sucrose, maltose, lactose, ribose, and mixtures thereof;
E. Surfactants which may be (but are not limited to): anionic surfactants contained anionic functional groups at their head, such as sulfate, sulfonate, phosphate, carboxylates, carboxylate salts; alkyl sulfates; alkyl-ether sulfates; carboxylate-based fluorosurfactants; cationic surfactants; primary, secondary, or tertiary amines; permanently charged quaternary ammonium salts; zwitterionic (amphoteric) surfactants; zwitterionic surfactants which cationic part is based on primary, secondary, or tertiary amines or quaternary ammonium cations; sultaines; betaines; phospholipids; zwitterionic surfactants of the tertiary amine oxides structural type; non-ionic surfactants; ethoxylates; fatty alcohol ethoxylates; alkylphenol ethoxylates (apes or apeos); fatty acid ethoxylates; special ethoxylated fatty esters and oils; ethoxylated amines and/or fatty acid amides; terminally blocked ethoxylates; fatty acid esters of polyhydroxy compounds; fatty acid esters of glycerol; fatty acid esters of sorbitol; fatty acid esters of sucrose; alkyl polyglucosides; ammonium lauryl sulfate; sodium lauryl sulfate; sodium dodecyl sulfate; sodium laureth sulfate; sodium lauryl ether sulfate; sodium myreth sulfate; docusate (dioctyl sodium sulfosuccinate) and their salts; perfluorooctanesulfonate (pfos); perfluorobutanesulfonate; alkyl-aryl ether phosphates; alkyl ether phosphates; sodium stearate; sodium lauroyl sarcosinate; perfluorononanoate; perfluorooctanoate; octenidine dihydrochloride; cetrimonium bromide (ctab); cetylpyridinium chloride (cpc); benzalkonium chloride (bac); benzethonium chloride (bzt); dimethyldioctadecylammonium chloride; dioctadecyldimethylammonium bromide (dodab); chaps (3-[(3-cholamidopropyl)dimethylammonio]-1-propanesulfonate); cocamidopropyl hydroxysultaine; cocamidopropyl betaine; phosphatidylserine; phosphatidylethanolamine; phosphatidylcholine; sphingomyelins; lauryldimethylamine oxide; myristamine oxide; narrow-range ethoxylates; octaethylene glycol monododecyl ether; pentaethylene glycol monododecyl ether; nonoxynols; triton x-100; polyethoxylated tallow amine; cocamide monoethanolamine; cocamide diethanolamine; poloxamers; glycerol monostearate; glycerol monolaurate; sorbitan monolaurate; sorbitan monostearate; sorbitan tristearate; tween(s), tween 20; tween 40; tween 60; tween 80; decyl glucoside; span(s), span 20, span 40, span 80; lauryl glucoside; octyl glucoside; poloxamer(s), poloxamer 188, poloxamer 407, polyoxyethylene stearate, myrj 52, deoxycholic acid, bile acids, pluronic(s), pluronic F-127, pluronic P85, pluronic f68, gelucire(s), gelucire 44/14, lecithins, polysorbates, polysorbate 80, plasdone-s630, pluronic-f68, inutec sp1, compritol 888 ato, tocopherol polyethylene glycol succinate, polyoxyethylated castor oil, polyoxyethylated glycerides, lauroyl macroglycerides, and mono- and di-fatty acid esters of low molecular weight polyethylene glycols; and mixtures thereof;
F. Acids and salts based on these acids which may be (but are not limited to): citric acid, tartaric acid, succinic acid, phosphoric acid, aminoacids, acetate(s), sodium acetate, alginate(s), sodium alginate, glycyrrhizic acid and their salts, and mixtures thereof;
G. Copolymers of the polymers listed in paragraph A, C including alternating copolymers, random copolymers, block copolymers, graft copolymers, cross-linked modifications which may be (but are not limited to): methacrylic acid and ethyl acrylate copolymers; methacrylic acid copolymers; vinylpyrrolidone-vinyl acetate copolymers (PVPVA); polyvinylpolypyrrolidone (PVPP); HPMC and PVA-PEG grafted copolymers; grafted polyvinylpyrrolidone-arabinogalactan copolymers; the graft copolymer has a) poly(vinyl acetate) and/or poly (vinyl alcohol) and/or poly(vinyl chloride) and poly(vinyl ester) on b) a polymer chain of polyethylene glycols, polyalkylene glycols, polypropylene glycols, polyisobutylene glycols orpolymeth-ylpentene glycols; graft copolymer polyvinyl acetate and/or hydrolysed polyvinyl acetate (polyvinyl alcohol) groups on a polyalkylene oxide (preferably polyethylene oxide); vinylpyrrolidone-vinyl acetate copolymers; vinylpyrrolidone-vinyl acetate copolymer-64+; vinylpyrrolidone-vinyl acetate VA 64; polymethacrylate-based copolymers includes anionic, cationic, and neutral copolymers based on methacrylic acid and methacrylic/acrylic esters their salts, esters or other derivatives and mixtures thereof;
H. Methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives of the substances specified in A-I, and their esters, salts, and any other chemical derivatives, and mixtures thereof;
J. Any combination of substances specified in items A-H;
but excluding Kollicoat IR and similar PVA-PEG graft copolymers;
wherein method of preparation is selected from:
a) dissolving solid dispersion (of FORMULA I) in solvent to form the liquid solubilized non-covalent complex (to form solutions, gels, colloids, sols, suspensions, etc.) under conditions sufficient to form the liquid solubilized non-covalent complex;
b) HOST substance is placed in a flask and completely dissolved in ethanol. Then a API, GUEST drug is added to the flask. Water is slowly added to the solution and stirred for several hours
c) when energy is supplied by any other means, such as (but not limited to) electromagnetic radiation, heat, mechanical action, sound, or in any of the ways described in the scientific and patent literature. As a non-limiting example, an example of exposure by mechanical and acoustic influence is given. Non-covalent complex obtained by the interaction of aqueous solutions of HOST substance and API, GUEST drug under conditions of mechano-acoustic and precipitation of the resulting complex with ethyl alcohol.

5. A method of manufacturing a pharmaceutical dosage form for treating viral infections (in humans or mammals, animals or birds),
or manufacturing or preparing a medical dosage form used as an antiviral agent,
comprising the steps of creating a solid dispersion or a non-covalent complex,
when the carrier (HOST) substance enhances the permeability of an active pharmaceutical ingredient (API, GUEST) by interacting with the biological membranes of an organism to improve the transfer of API, GUEST molecules, ions, or metabolites across these membranes
(resulting in a pharmaceutical dosage form with increased therapeutic efficacy);
and wherein the improved permeability makes it possible to rapidly achieve the required concentrations (EC (Effective Concentration) or IC (Inhibitory Concentration)) in the patient's organs and tissues.

6. The non-covalent complex or solid dispersion of claims 1-5 wherein the non-covalent complex or solid dispersion has at least one of the altered properties enhancing therapeutic efficacy (compared to pure ivermectin or pure avermectin):
1) has a higher bioavailability than pure ivermectin or pure avermectin;
2) has a higher permeability than pure ivermectin or pure avermectin;
3) has a lower toxicity or cytotoxicity than pure ivermectin or pure avermectin;
4) has a different pharmacokinetic parameters than pure ivermectin or pure avermectin;
5) has a improved solubility than pure ivermectin or pure avermectin.

7. The method of claims 1-5 wherein the non-covalent complex or solid dispersion has at least one of the altered properties enhancing therapeutic efficacy (compared to pure ivermectin or pure avermectin):
1) has a higher bioavailability than pure ivermectin or pure avermectin;
2) has a higher permeability than pure ivermectin or pure avermectin;
3) has a lower toxicity or cytotoxicity than pure ivermectin or a pure vermectin;
4) has a different pharmacokinetic parameters than pure ivermectin or pure avermectin;
5) has a improved solubility than pure ivermectin or pure avermectin.

8. The non-covalent complex or solid dispersion of claims 1-7 wherein the non-covalent complex or solid dispersion is combined with at least one of the following:
1) modulators of P-glycoprotein;
2) modulators of CYP3A4;
3) synergist of ivermectin, avermectin;
4) quercetin and/or dihydroquercetin and/or vitamin C;
5) additional therapeutic agent active (in vitro, in vivo, in silico) against infectious viruses;
6) additional therapeutic agent that increases the antiviral activity of the non-covalent complex;
7) additional therapeutic agent that decreases the hepatotoxicity of the non-covalent complex;
8) additional therapeutic agent selected from agents listed in paragraph [0081](IV.I. Section) or derivatives thereof;
9) niclosamide and/or with nitazoxanide and/or with melatonin;
10) mebeverine hydrochloride;
but excluding combination with an antibiotic (tetracycline, doxycycline or azithromycin) and zinc.

9. The method of claims 1-7 wherein the non-covalent complex or solid dispersion is combined with at least one of the following:
1) modulators of P-glycoprotein;
2) modulators of CYP3A4;
3) synergist of ivermectin, avermectin;
4) quercetin and/or dihydroquercetin and/or vitamin C;
5) additional therapeutic agent active (in vitro, in vivo, in silico) against infectious viruses;
6) additional therapeutic agent that increases the antiviral activity of the non-covalent complex;
7) additional therapeutic agent that decreases the hepatotoxicity of the non-covalent complex;
8) additional therapeutic agent selected from agents listed in paragraph [0081](IV.I. Section) or derivatives thereof;
9) niclosamide and/or with nitazoxanide and/or with melatonin;
10) mebeverine hydrochloride;
but excluding combination with an antibiotic (tetracycline, doxycycline or azithromycin) and zinc.

10. The non-covalent complexes or solid dispersion of claims 1-9 when non-covalent complexes or solid dispersions are used
1) in manufacturing a pharmaceutical dosage form to treat viral infections in a human or a mammal or an animal or a bird;
2) for the reduction of viral load of a human or a mammal or an animal or a bird;
3) as anticancer agent, anticancer medical dosage form;
4) for a wide range of active agents of the group of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha 2-adrenergic agonists, analgesics, antipyretics and anti-inflammatory agents, androgens, anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatelet agents, antiprogestins, antithyroid agents, anti-tussives, antiviral agents, a typical antidepressants, azaspirodecanediones, barbituates, benzodiazepines, benzothiadiazides, beta-adrenergic agonists, beta-adrenergic antagonists, selective beta 1-adrenergic antagonists, selective beta 2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, haematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates, opiod analgesics and antagonists, pancreatic enzymes, pheno thiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like, alone or in combination. Although extensive, this list is not intended to be comprehensive.

11. The method of claims 1-9 when non-covalent complexes or solid dispersion are used for manufacturing a pharmaceutical dosage form to treat viral infections in a human or a mammal or an animal or a bird
or for use in manufacturing or preparing a medical dosage form used as an antiviral agent.

12. The method of claims 1-11 when:
A) a therapeutically effective amount of a stabilized aqueous formulation is administered which comprises avermectin or ivermectin, solvent, surface active agent and co-solvent;
B) a therapeutically effective amount of a stabilized formulation is administered which comprises avermectin or ivermectin solutions in fat/oil or avermectin or ivermectin emulsion;
C) a therapeutically effective amount of compound of FORMULA IV is administered in the form of a solution or emulsion or suspension comprising:
1) a first pharmaceutical composition comprising a compound of FORMULA 1-111, and
2) at least one of the following components:
a) oil phase containing vegetable and/or animal fats;
b) one or more surfactants;
c) one or more solvents;
d) one or more gelling agents;
D) a therapeutically effective amount of compound of FORMULA V is administered in the form of a food product or beverage, or dietary supplement;
E) a pharmaceutically acceptable diluents, carrier, or excipient is co-administered;
F) a solid dispersion is administered instead of a non-covalent complex.

13. The method of claims 1-12 when the dosage of non-covalent complex or solid dispersion for the respective virus exceeds:
1) IC5—5% of maximal inhibitory concentration for ivermectins or avermectins;
2) IC50—half maximal inhibitory concentration for ivermectins or avermectins;
3) IC90—90% of maximal inhibitory concentration for ivermectins or avermectins.

14. The non-covalent complex or solid dispersion of claims 1-13 when the carrier (HOST) is a polymer, biopolymer or synthetic polymer, or methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives, esters and ethers, salts, and any other chemical derivatives, and mixtures thereof.

15. The method of claims 1-14 when the carrier (HOST) is a polymer, biopolymer or synthetic polymer, or methoxylated, ethoxylated, esterificated, carboxylated, alkoxylated, acetylated, hydroxylated, hydrated, decarboxylated, amide, oxidized, sulfated, aminoacid derivatives, fermented, thermally modified, chemically modified, acid modified derivatives, esters and ethers, salts, and any other chemical derivatives, and mixtures thereof.

16. The method of claims 1-15 when the mass ratio of ivermectins or avermectins to a polymer, biopolymer or synthetic polymer (HOST) is from 1:1 to 1:10000.

17. The non-covalent complex or solid dispersion of claims 1-15 when the mass ratio of ivermectins or avermectins to a polymer, biopolymer or synthetic polymer (HOST) is from 1:1 to 1:10000.

18. The method of claims 1-17 when non-covalent complex or solid dispersion is administered in composition that can be formulated in any way and can be applied in a variety of forms including polymeric nanoparticles (PNPs), liposomes, micelles, niosomes, solid lipid nanoparticles (SLNs), nanostructured lipid carriers (NLCs), nanoemulsions, emulsions, self-nanoemulsifying drug delivery systems (SNEDDS), nanocrystals, mesoporous silica nanoparticles (MSNs) and dendrimers; and/or using methods of physical modification, chemical modification or miscellaneous methods modification.

19. The method of claims 1-18 when used to treat disease in a human or a mammal or an animal or a bird.
